(19) United States
(12) Patent Application Publication
(10) Pub. No.: US 2014/0275108 A1
(43) Pub. Date: Sep. 18, 2014

(54) NOVEL BENZENESULFONAMIDE COMPOUNDS, METHOD FOR SYNTHESIZING SAME, AND USE THEREOF IN MEDICINE AS WELL AS IN COSMETICS

(71) Applicant: GALDERMA RESEARCH & DEVELOPMENT, Biot (FR)

(72) Inventors: Carine MOUNIER, Mougins (FR); Isabelle Carlavan, Grasse (FR); Jerome Aubert, Grasse (FR); André Jomard, Saint Vallier de Thiey (FR); Patricia Rossio, Grasse (FR)

(73) Assignee: GALDERMA RESEARCH & DEVELOPMENT, Biot (FR)

(21) Appl. No.: 13/841,524

(22) Filed: Mar. 15, 2013

(51) Int. Cl. C07D 295/15; C07D 471/04; C07D 209/12; C07D 261/20; C07D 215/14; C07D 213/30

(52) U.S. Cl. CPC C07D 295/15; C07D 215/14; C07D 213/30; C07D 209/12; C07D 261/20; C07D 471/04
USPC 514/253.06; 514/252.12; 435/371; 514/253.01; 514/254.09; 514/254.04; 514/253.04

(57) ABSTRACT

Provided herein are novel benzenesulfonamide compounds having a structure of formula (I) below:

[chemical structure]

as well as to the method for synthesizing same and to the use thereof in pharmaceutical compositions to be used in human or veterinary medicine, as well as to the use thereof. Also provided herein are methods of using the disclosed pharmaceutical compositions for the treatment of neuroinflammatory skin diseases such as acne.

### FIELD

The disclosure relates to novel benzenesulfonamide compounds corresponding to general formula (I) below:

[chemical structure]

and also to the process for synthesizing same and to the use thereof in pharmaceutical compositions intended for use in human or veterinary medicine.

The compounds disclosed herein act as inhibitors of TNFα-converting enzyme, also known as TACE. They are consequently of use in the treatment of diseases for which reducing TNFα production is of great interest.

The present disclosure also relates to the use of the compounds corresponding to general formula (I) in cosmetic compositions.

### BACKGROUND

Adamalysins (“ADAM” or A Disintegrin and Metalloproteinase) are a subfamily of zinc metalloendopeptidase enzymes. Their ectodomain comprises a protease domain, the activation of which is zinc-dependent, a disintegrin domain and a cysteine-rich domain. To date, at least 30 different ADAMs have been identified, of which the first characterized was ADAM17, also known as TACE (TNFα-converting enzyme) [Gueydan C et al. Med. Sci 1997, 13, 83-88; Black R. A et al. Nature 1997, 385:729-733; Moss et al. Nature 1997, 385:733-736]. The TACE mRNA is present in many tissues and more particularly in monocytes, macrophages, and T lymphocytes, but also in keratinocytes for example.

TACE is responsible for the cleavage of pro-TNFα, a 26 kDa membrane protein, so as to result in the release of biologically active soluble TNFα, a 17 kDa protein [Schlondorff et al. Biochem. J. 2000, 347, 131-138]. The soluble TNFα released by the cell is capable of acting on sites very remote from the site of synthesis.

TNFα is involved in a large number of pro-inflammatory biological processes [Aggarwal et al, Eur. Cytokine Netw., 1996, 7: 93-124]. Several pharmacological and clinical studies have shown in an obvious manner that blocking the effects of TNFα with specific anti-TNFα antibodies or anti-TNFα biologicals (Etanercept, Adalimumab, Infliximab) is beneficial in the treatment of autoimmune diseases such as rheumatoid arthritis [Feldman et al. Lancet, 1994, 344, 1105], non-insulin-dependent diabetes mellitus [Lohmander L. S et al. Arthritis Rheum, 1993, 36, 1214-1222], or Crohn's disease [MacDonald et al. Clin. Exp. Immunol. 1990, 81, 301].

TNFα also plays a fundamental role during the inflammatory phenomenon triggered in psoriasis lesions. Serum TNFα levels are elevated in psoriatic patients [Mussi A et al. J. Biol. Regul. Homeost Agents, 1997, 11, 115-118]; TNFα levels are also elevated in the actual psoriasis plaques [Bonifati C. et al. Clin. Exp. Dermatol., 1994, 19, 383-387]. The key cells in the physiopathology of psoriasis are keratinocytes, dendritic cells, and certain T lymphocytes. The interaction between these families of cells results in an inflammatory cascade that leads to the characteristic psoriasis lesions with release of TNFα (Kupper T S, N. Engl. J. Med, 2003, 349, 1987-1990. Clinical studies for the treatment of moderate to severe plaque psoriasis with anti-TNFα biologicals (Etanercept, Adalimumab, Infliximab) have demonstrated their efficacy both on psoriasis lesions and on the quality of life of the patients (Ortonne J P, Annales de dermatologie et de venereologie {Annals of dermatology and venereology}, 2005, 132 (8-9 pt2), 4S6-9 and 2005, 132, 9S01-9S70).

Thus, compounds which inhibit TNFα production are of great interest for the treatment of inflammatory diseases, in particular inflammatory skin diseases, such as acne, and other diseases involving TNFα release.

Acne is a common skin disease, characterized by areas of skin with seborrhea (scaly red skin), comedones (blackheads and whiteheads), papules (pinheads), pustules (pimples), nodules (large papules) and possibly scarring (Adityan et al., Indian J Dermatol Venereol Leprol 75 (3): 323-6, 2009). Multi-factors contribute to the development acne, such as plugging of the hair follicle with abnormally cohesive desquamated cells, proliferation and colonization of bacteria (e.g., Propionibacterium acnes), local inflammation, and abnormalities in follicular keratinization and sebum production. Antibiotics have been used to treat acne but have limited use. Accordingly, there is a need to develop new agents for treating acne.

It has been reported by Kang et al. that transcription factors nuclear factor-KBand activator protein-1 were activated in acne lesions with consequent elevated expression of their target gene products, inflammatory cytokines and matrix-degrading metalloproteinases (Kang et al., American Journal of Pathology, Vol. 166 (6): 1691-1699, 2005). These elevated genes are molecular mediators of inflammation and collagen degradation in acne lesions in vivo. Id.

### SUMMARY

Provided herein are compounds having formula (I) below:

[chemical structure]

in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an alkyl radical, a substituted alkyl radical, an alkenyl radical, a substituted alkenyl radical, an alkynyl radical, a substituted alkynyl radical, an aryl radical, a substituted aryl radical, an aralkyl radical, a substituted aralkyl radical, a heterocyclic radical, a substituted heterocyclic radical, a cycloalkyl radical, a substituted cycloalkyl radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical;
n can take the values of 0, 1, 2, or 3; and,
salts of the compounds having the structure of formula (I) including their addition salts with a pharmaceutically acceptable acid, and their addition salts with a pharmaceutically acceptable base; and,
enantiomers of the compounds having the structure of formula (I).

Also provided herein are compositions comprising a compound having formula (I), a salt thereof, or an enantiomer thereof and a carrier. In one embodiment, the composition is a pharmaceutical composition and the carrier is a pharmaceutically-acceptable carrier. In another embodiment, the pharmaceutical composition comprises a therapeutically-effective amount of a compound having formula (I), a salt thereof, or an enantiomer thereof and a pharmaceutically-acceptable carrier for treating a disease or condition.

Provided herein are methods of using the compounds to treat inflammatory diseases and conditions. In particular, the pharmaceutical compositions provided herein are effective in treating inflammatory skin diseases, such as acne, psoriasis, atopic dermatitis, and psoriatic arthritis. The methods comprise administering an effective amount of a pharmaceutical composition disclosed herein to a subject in need thereof.

### BRIEF DESCRIPTION OF THE DRAWINGS

FIG. 1 shows cutaneous inflammation induced by P. acnes using the mouse ear edema model.

FIGS. 2A and 2B show the inhibition of TNFα (FIG. 2A) and IL-6 (FIG. 2B) secretion by Compound D using skin cells from the mouse ear edema model.

### DETAILED DESCRIPTION

Provided herein are novel compounds that inhibit the TACE enzyme (TNFα-converting enzyme) and, as a result, inhibit the secretion of soluble TNFα (active form of TNFα) by cells. These compounds are therefore potentially active ingredients for the treatment of pathological conditions that involve a decrease or an inhibition of TNFα production. These compounds are useful for the treatment of inflammatory diseases.

By way of illustration, and in a nonlimiting manner, these pathological conditions are, for example, septic shock, hemodynamic shock, malaria, inflammatory bowel disease (IBD) such as Crohn's disease and ulcerative colitis, inflammatory bone diseases, mycobacterial infection, meningitis, fibrotic disease, cardiac disease, ischemic attack, transplant rejection, cancer, atherosclerosis, obesity, disease involving angiogenesis phenomena, autoimmune disease, osteoarthritis, rheumatoid arthritis, ankylosing spondylitis, juvenile chronic arthritis, multiple sclerosis, HIV, non-insulin-dependent diabetes mellitus, allergic disease, asthma, chronic obstructive pulmonary disease (COPD), and occular inflammation.

The compounds provided herein are useful for the treatment of an inflammatory skin disease, such as, acne, psoriasis, atopic dermatitis, and psoriatic arthritis.

These molecules are also potential active ingredients for the treatment of neurological pathological conditions that are inflammatory in nature, for which reducing TNFα production would be of great interest. The pathological conditions listed hereinafter in a nonlimiting manner are, for example, Alzheimer's disease, Parkinson's disease, parkinsonian disorder, amyotrophic lateral sclerosis, an autoimmune disease of the nervous system, autonomic disease of the nervous system, dorsal pain, cerebral edema, cerebrovascular disorder, dementia, nervous system nerve fiber demyelinating autoimmune disease, diabetic neuropathy, encephalitis, encephalomyelitis, epilepsy, chronic fatigue syndrome, giant cell arteritis, Guillain-Barré syndrome, headache, multiple sclerosis, neuralgia, peripheral nervous system disease, polyneuropathy, polyradiculoneuropathy, radiculopathy, respiratory paralysis, spinal cord disease, Tourette's syndrome, central nervous system vasculitis, Huntington's disease, and stroke.

A large variety of TACE inhibitors are already known as indicated below. However, a large number of these inhibitors do not act selectively on the TACE enzyme compared with other enzymes of the family of ADAMs and/or of matrix metalloproteinases (MMPs).

As it happens, the nonselective inhibition of these enzyme families induces adverse side effects observed in vivo. For example, the inhibition of MMP-1 (collagenase-1) has been associated with musculoskeletal toxicity problems.

As a nonselective inhibitor, mention may also be made of Apratastat, a known inhibitor tested clinically in phase 2 for the treatment of rheumatoid arthritis (Curr Opin Investig Drugs. 2006 Nov. 7(11), 1014-1019). This inhibitor is not selective for the TACE enzyme compared with certain MMPs (WO 00/44709; page 251, table 10, Example 61).

Other TACE inhibitors, which are also known and are part of the same family as Apratastat, namely that of cyclic benzenesulfonamide derivatives, have been described in WO 00/44709 and WO 97/18194. Other patents (WO 96/00214, WO 97/22587) claim MMP and/or TACE inhibitors for which the benzenesulfonamide part is separated from the hydroxamic acid function by a single carbon atom. Publications describing MMP inhibitors of this type more broadly are also the publication by MacPherson et al. J. Med. Chem. 1997, 40, 2525 and the publication by Tamura et al. J. Med. Chem. 1998, 41, 640. Other examples of MMP/TACE inhibitors for which the sulfonamide function is separated from the hydroxamic acid by a series of two carbon atoms forming a ring are described in patents WO 98/16503, WO 98/16506, WO 98/16514 and WO 98/16520. Other examples of MMP inhibitors for which the sulfonamide function is separated from the hydroxamic acid by a series of two carbon atoms are also described in WO 2008/045671.

As it happens, the applicant has now discovered, unexpectedly and surprisingly, that novel compounds having the structure of formula (I) exhibit a very good TACE-inhibiting activity, and in particular inhibit the TACE enzyme selectively compared with other ADAMs and MMPs.

Provided herein are compounds having formula (I) below:

[chemical structure]

in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an alkyl radical, a substituted alkyl radical, an alkenyl radical, a substituted alkenyl radical, an alkynyl radical, a substituted alkynyl radical, an aryl radical, a substituted aryl radical, an aralkyl radical, a substituted aralkyl radical, a heterocyclic radical, a substituted heterocyclic radical, a cycloalkyl radical, a substituted cycloalkyl radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical;
n can take the values of 0, 1, 2, or 3; and,
salts of the compounds having the structure of formula (I) including their addition salts with a pharmaceutically acceptable acid, and their addition salts with a pharmaceutically acceptable base; and,
enantiomers of the compounds having the structure of formula (I).

Among the addition salts of the compounds having the structure of formula (I) with a pharmaceutically acceptable acid, mention is made of the salts with an organic acid or with an inorganic acid.

The suitable inorganic acids are, for example, hydrohalic acids such as hydrochloric acid or hydrobromic acid, sulfuric acid, nitric acid, and phosphoric acid.

The suitable organic acids are, for example, acetic acid, trifluoroacetic acid, trichloroacetic acid, propionic acid, glycolic acid, pyruvic acid, succinic acid, benzoic acid, cinnamic acid, mandelic acid, methanesulfonic acid, para-toluenesulfonic acid, salicylic acid, picric acid, citric acid, oxalic acid, tartaric acid, malonic acid, maleic acid, camphorsulfonic acid, and fumaric acid.

Among the addition salts of the compounds having the structure formula (I) with a pharmaceutically acceptable base, mention is made of the salts with an organic base or with an inorganic base.

The inorganic bases are, for example, potassium hydroxide, sodium hydroxide, lithium hydroxide, or calcium hydroxide.

The suitable organic bases comprise amines and amino acids. Among the amines, mention may, for example, be made of aliphatic or aromatic, primary, secondary, or tertiary amines, such as methylamine, ethylamine, ethanolamine, propylamine, isopropylamine, the 4 isomers of butylamine, dimethylamine, diethylamine, diethanolamine, dipropylamine, diisopropylamine, di-n-butylamine, pyrrolidine, piperidine, morpholine, diethanolphenylamine, trimethylamine, triethylamine, tripropylamine, quinuclidine, pyridine, quinoline, or isoquinoline.

Among the amino acids, mention may, for example, be made of lysine, arginine, and ornithine.

As used herein, the term “lower alkyl radical” denotes a linear or branched, saturated hydrocarbon-based chain containing from 1 to 4 carbon atoms.

As used herein, the term “alkyl radical” denotes a linear or branched, saturated hydrocarbon-based chain containing from 1 to 10 carbon atoms.

As used herein, the term “alkenyl radical” denotes a linear or branched, unsaturated hydrocarbon-based chain containing from 2 to 10 carbon atoms and comprising one or more double bonds.

As used herein, the term “alkynyl radical” denotes a linear or branched, unsaturated hydrocarbon-based chain containing from 2 to 10 carbon atoms and comprising one or more triple bonds.

As used herein, the term “substituted alkyl radical” denotes a linear or branched, saturated hydrocarbon-based chain containing from 1 to 10 carbon atoms and substituted with one or more radicals chosen from a halogen atom, an alkoxy radical, and a hydroxyl radical.

As used herein, the term “substituted alkenyl radical” denotes a linear or branched, unsaturated hydrocarbon-based chain containing from 2 to 10 carbon atoms, comprising one or more double bonds and substituted with one or more radicals chosen from a halogen atom, an alkoxy radical, and a hydroxyl radical.

As used herein, the term “substituted alkynyl radical” denotes a linear or branched, unsaturated hydrocarbon-based chain containing from 2 to 10 carbon atoms, comprising one or more triple bonds and substituted with one or more radicals chosen from a halogen atom, an alkoxy radical, and a hydroxyl radical.

As used herein, the term “cycloalkyl” denotes a cyclic saturated hydrocarbon-based chain containing from 3 to 7 carbon atoms.

As used herein, the term “substituted cycloalkyl” denotes a cyclic saturated hydrocarbon-based chain containing from 3 to 7 carbon atoms and substituted with one or more radicals chosen from a halogen atom, an alkoxy radical, and a hydroxyl radical.

As used herein, the term “aryl radical” denotes an aromatic hydrocarbon-based ring or two fused aromatic hydrocarbon-based rings.

The preferred aryl radicals are chosen from phenyl and naphthyl radicals.

As used herein, the term “substituted aryl radical” denotes an aromatic hydrocarbon-based ring or two fused aromatic hydrocarbon-based rings which is (are) substituted with one or more groups of atoms chosen from an alkyl, an alkoxy, an aryl, a halogen, a hydroxyl, a cyano, a trifluoromethyl, and a nitro.

As used herein, the term “aralkyl radical” denotes an alkyl substituted with an aryl.

As used herein, the term “substituted aralkyl radical” denotes an alkyl substituted with a substituted aryl.

As used herein, the term “heterocyclic radical” denotes a saturated or unsaturated, cyclic or polycyclic hydrocarbon-based chain comprising one or more heteroatoms chosen from O, S, and N.

As used herein, the term “substituted heterocyclic radical” denotes a heterocyclic radical substituted with one or more groups of atoms chosen from an alkyl, an alkoxy, a halogen, a hydroxyl, a cyano, a trifluoromethyl, and a nitro.

As used herein, the term “heteroaryl radical” denotes an aromatic heterocyclic radical, i.e. a cyclic or polycyclic aromatic hydrocarbon-based chain, comprising one or more heteroatoms chosen from O, S, and N.

As used herein, the term “substituted heteroaryl radical” denotes a heteroaryl radical substituted with one or more groups of atoms chosen, for example, from an alkyl, an alkoxy, an aryl, a substituted aryl, a halogen, a hydroxyl, a cyano, a trifluoromethyl, and a nitro.

As used herein, the term “heteroaralkyl radical” denotes an alkyl radical substituted with a heteroaryl radical.

As used herein, the term “substituted heteroaralkyl radical” denotes a heteroaralkyl radical substituted with one or more groups of atoms chosen from an alkyl, an alkoxy, a halogen, a hydroxyl, a cyano, a trifluoromethyl, and a nitro.

As used herein, the term “alkoxy radical” denotes an oxygen atom substituted with an alkyl radical.

As used herein, the term “halogen atom” denotes a fluorine, chlorine, bromine, or iodine atom.

Among the compounds having the structure of formula (I), mention may in particular be made of the following compounds:
1) 3-[(4-but-2-ynyloxybenzenesulfonyl)methylamino]-N-hydroxy-2-(4-methanesulfonylpiperazin-1-yl)propionamide,
2) (S)-3-(4-but-2-ynyloxybenzenesulfonylamino)-N-hydroxy-2-(4-methanesulfonylpiperazin-1-yl)propionamide,
3) (S)-3-(4-benzyloxybenzenesulfonylamino)-N-hydroxy-2-(4-methanesulfonylpiperazin-1-yl)propionamide,
4) (S)-3-[(4-benzyloxybenzenesulfonyl)methylamino]-N-hydroxy-2-(4-methanesulfonylpiperazin-1-yl)propionamide,
5) (S)—N-hydroxy-2-(4-methanesulfonylpiperazin-1-yl)-3-[4-(2-methylquinolin-4-ylmethoxy)benzenesulfonylamino]propionamide,
6) (S)—N-hydroxy-2-(4-methanesulfonylpiperazin-1-yl)-3-[4-(naphthalen-1-ylmethoxy)benzenesulfonylamino]propionamide,
7) (S)—N-hydroxy-2-(4-methanesulfonylpiperazin-1-yl)-3-(4-propoxybenzenesulfonylamino)propionamide,
8) (S)-3-[4-(3-cyanobenzyloxy)benzenesulfonylamino]-N-hydroxy-2-(4-methanesulfonylpiperazin-1-yl)propionamide,
9) (S)-3-[4-(4-cyanobenzyloxy)benzenesulfonylamino]-N-hydroxy-2-(4-methanesulfonylpiperazin-1-yl)propionamide,
10) benzyl 4-{(S)-1-hydroxycarbamoyl-2-[4-(2-methylquinolin-4-ylmethoxy)benzenesulfonylamino]ethyl}piperazine-1-carboxylate,
11) (S)—N-hydroxy-2-(4-methanesulfonylpiperazin-1-yl)-3-[4-(2-phenylpyridin-4-ylmethoxy)benzenesulfonylamino]propionamide,
12) (R)—N-hydroxy-2-(4-methanesulfonylpiperazin-1-yl)-3-[4-(2-methylquinolin-4-ylmethoxy)benzenesulfonylamino]propionamide,
13) (S)—N-hydroxy-3-[4-(2-methylquinolin-4-ylmethoxy)benzenesulfonylamino]-2-piperazin-1-yl-propionamide,
14) (S)—N-hydroxy-2-(4-methanesulfonylpiperazin-1-yl)-3-[4-(2-methylquinolin-4-ylmethoxy)benzenesulfonylamino]propionamide hydrochloride,
15) tert-butyl 3-{4-[(S)-2-hydroxycarbamoyl-2-(4-methanesulfonylpiperazin-1-yl)ethylsulfamoyl]phenoxymethyl}-2-methylindole-1-carboxylate di(trifluoroacetate),
16) (S)—N-hydroxy-2-(4-methanesulfonylpiperazin-1-yl)-3-[4-(quinolin-4-ylmethoxy)benzenesulfonylamino]propionamide,
17) (S)-2-(4-benzylpiperazin-1-yl)-N-hydroxy-3-[4-(2-methylquinolin-4-ylmethoxy)benzenesulfonylamino]propionamide,
18) (S)-2-[4-(4-fluorobenzyl)piperazin-1-yl]-N-hydroxy-3-[4-(2-methylquinolin-4-ylmethoxy)benzenesulfonylamino]propionamide,
19) (S)-2-(4-ethylpiperazin-1-yl)-N-hydroxy-3-[4-(2-methylquinolin-4-ylmethoxy)benzenesulfonylamino]propionamide,
20) (S)—N-hydroxy-3-[4-(2-methylquinolin-4-ylmethoxy)benzenesulfonylamino]-2-[4-(4-trifluoromethyl-benzyl)piperazin-1-yl]propionamide,
21) (S)—N-hydroxy-2-[4-(4-methylbenzyl)piperazin-1-yl]-3-[4-(2-methylquinolin-4-ylmethoxy)benzenesulfonylamino]propionamide,
22) (S)-3-[4-(benzoisoxazol-3-ylmethoxy)benzenesulfonylamino]-N-hydroxy-2-(4-methanesulfonylpiperazin-1-yl)propionamide,
23) (S)—N-hydroxy-2-(4-isobutyrylpiperazin-1-yl)-3-[4-(2-methylquinolin-4-ylmethoxy)benzenesulfonylamino]propionamide,
24) (S)—N-hydroxy-2-[4-(2-methylpropane-1-sulfonyl)piperazin-1-l]-3-[4-(2-methylquinolin-4-ylmethoxy)benzenesulfonylamino]propionamide,
25) (S)—N-hydroxy-2-(4-methanesulfonylpiperazin-1-yl)-3-[4-(2-trifluoromethylpyrazolo[1,5-a]pyridin-3-ylmethoxy)benzenesulfonylamino]propionamide,
26) (S)—N-hydroxy-3-[4-(2-methylquinolin-4-ylmethoxy)benzenesulfonylamino]-2-[4-(propane-2-sulfonyl)piperazin-1-yl]propionamide,
27) (S)-2-(4-benzylpiperazin-1-yl)-N-hydroxy-3-[4-(2-trifluoromethylpyrazolo[1,5-a]pyridin-3-ylmethoxy)benzenesulfonylamino]propionamide,
28) (S)-2-(4-acetylpiperazin-1-yl)-N-hydroxy-3-[4-(2-methylquinolin-4-ylmethoxy)benzenesulfonylamino]propionamide,
29) (S)—N-hydroxy-2-(4-methanesulfonylpiperazin-1-yl)-3-{propyl-[4-(quinolin-4-ylmethoxy)benzenesulfonyl]amino}propionamide,
30) (S)-2-(4-benzenesulfonylpiperazin-1-yl)-N-hydroxy-3-[4-(pyrazolo[1,5-a]pyridin-3-ylmethoxy)benzenesulfonylamino]propionamide,
31) (S)-2-(4-benzylpiperazin-1-yl)-N-hydroxy-3-[4-(1-methylpiperidin-4-ylmethoxy)benzenesulfonylamino]propionamide,
32) (S)-2-[4-(4-fluorobenzoyl)piperazin-1-yl]-N-hydroxy-3-[4-(3-m-tolyl-propoxy)benzenesulfonylamino]propionamide,
33) (S)—N-hydroxy-3-[4-(2-methylnaphthalen-1-ylmethoxy)benzenesulfonylamino]-2-(4-propionylpiperazin-1-yl)propionamide,
34) (S)—N-hydroxy-3-[4-(4-methylpentyloxy)benzenesulfonylamino]-2-(4-phenylacetylpiperazin-1-yl)propionamide,
35) (S)—N-hydroxy-2-(4-methanesulfonylpiperazin-1-yl)-3-[4-(2-methylpyridin-4-ylmethoxy)benzenesulfonylamino]propionamide,
36) (S)-2-(3-acetylimidazolidin-1-yl)-N-hydroxy-3-[4-(2-methylquinolin-4-ylmethoxy)benzenesulfonylamino]propionamide,
37) (S)-3-[4-(3,5-dimethylbenzyloxy)benzenesulfonylamino]-N-hydroxy-2-imidazolidin-1-yl-propionamide,
38) (S)—N-hydroxy-2-(4-methanesulfonyl-[1,4]diazepan-1-yl)-3-[4-(2-methylquinolin-4-ylmethoxy)benzenesulfonylamino]propionamide,
39) (S)-2-(4-benzyl-[1,4]diazepan-1-yl)-N-hydroxy-3-[4-(2-methylquinolin-4-ylmethoxy)benzenesulfonylamino]propionamide,
40) (S)-2-[1,4]diazocan-1-yl-N-hydroxy-3-[4-(2-methylquinolin-4-ylmethoxy)benzenesulfonylamino]propionamide,
41) (S)—N-hydroxy-3-[4-(2-methylbenzofuran-3-ylmethoxy)benzenesulfonylamino]-2-[4-(propane-2-sulfonyl)piperazin-1-yl]propionamide, and
42) (S)-2-(4-benzylpiperazin-1-yl)-N-hydroxy-3-[4-(2-isopropyl-1H-indol-3-ylmethoxy)benzenesulfonylamino]propionamide.

The compounds of general formula (I) are prepared according to the reaction scheme of scheme 1 presented below.

[chemical structure]

[chemical structure]

According to scheme 1, the compounds (3) are obtained by reaction between the amino acid (1) H-DAP(Boc)-OMe.HCl or H-(D)-DAP(Boc)-OMe.HCl, and the compound (2) (commercial or prepared beforehand) in the presence of an organic tertiary base such as diisopropylethylamine or triethylamine at a temperature of between 60° C. and 120° C. The compounds (4) are obtained by deprotection of the amine function of compounds (3) according to conventional methods such as, for example, the use of a solution of hydrochloric acid in isopropanol.

A reaction between the compound (4) and 4-hydroxybenzenesulfonyl chloride O-protected with a benzyl group for example (P=CH₂-Ph) (5) in the presence of a tertiary amine such as, for example, triethylamine in dichloromethane, produces the compound (6). An N-alkylation of the sulfonamide function can then be carried out by reaction with an alkyl halide in the presence of a base such as, for example, potassium carbonate in a solvent such as DMF, so as to give the derivative (7). The compound (8) is obtained by deprotection according to methods known by those skilled in the art for deprotecting a phenol function. The compound (9) is obtained by alkylation of the phenol function of the compound (8) by reaction with an alkyl halide in the presence of a base such as, for example, cesium carbonate in acetone, or via a Mitsunobu reaction with a primary alcohol derivative in the presence of triphenylphosphine and of diisopropyl azodicarboxylate for example. The compound (10) is obtained via a saponification reaction in the presence of a base such as lithium hydroxide in the presence of water and of tetrahydrofuran for example. In a final step, the compound (11) is obtained by coupling between O-(tert-butyldimethylsilyl)hydroxylamine, for example, and the derivative (10) under conventional peptide coupling conditions, using, for example, 1-(3-dimethylaminopropyl)-3-ethylcarbodiimide hydrochloride, hydroxybenzotriazole, or TBTU as coupling agents, and triethylamine or diisopropylethylamine as base, in a solvent such as dichloromethane or dimethylformamide. The deprotection of the silylated hydroxamic acid intermediately formed is carried out in situ or by washing with a slightly acidic aqueous solution, so as to give the compound (11).

Another alternative for obtaining the compound (11) is presented in scheme 2 below.

[chemical structure]

According to the synthesis scheme of scheme 2, the derivative (3) can optionally be alkylated in the presence of a base such as sodium hydride and of an alkyl halide in dimethylformamide, for example, so as to give the compound (12), from which the compound (13) is obtained according to conventional methods for deprotecting amines, for instance the use of a solution of hydrochloric acid in isopropanol.

The compound (14) is prepared beforehand from the commercially available 4-hydroxybenzenesulfonic acid sodium salt by alkylation with an alkyl halide in the presence of a base such as sodium hydroxide, for example, in a mixture of solvents such as isopropanol and water, for example. The compound (15) is then obtained by reacting the compound (14) with oxalyl chloride in the presence of dimethylformamide in dichloromethane, for example.

The derivative (9) is obtained by reaction between the compounds (13) and (15) in the presence of a base such as triethylamine in dichloromethane, for example.

An alternative synthesis pathway for obtaining the compound (11) is also presented in scheme 3 below.

[chemical structure]

According to scheme 3, the compound (17) is obtained by reacting the amino acid (1) H-DAP(Boc)-OMe.HCl or H-(D)-DAP(Boc)-OMe.HCl, and the compound (16) (prepared beforehand by reacting bis(2-chloroethyl)amine, for example, and benzyl bromide in the presence of potassium carbonate in acetonitrile) in the presence of an organic tertiary base such as diisopropylethylamine at a temperature of approximately 120° C. After deprotection of the amine function, the compound (18) is condensed with sulfonyl chloride (15) so as to give the derivative (19). An N-alkylation of the sulfonamide function can then be carried out by reaction with an alkyl halide in the presence of a base such as, for example, potassium carbonate in a solvent such as DMF, so as to give the derivative (20). The compound (21) is obtained according to the conventional conditions for hydrogenation of the compound (20) in the presence of palladium-on-carbon in a solvent such as ethanol for example. The compound (9) is obtained according to the conventional synthesis methods, for example, by reacting the compound (21) with an acyl chloride or a sulfonyl chloride in the presence of triethylamine, or by reacting with an alkyl halide in the presence of a base such as sodium hydride, for example. The compound (10) is obtained via a saponification reaction in the presence of a base such as lithium hydroxide in the presence of water and of tetrahydrofuran, for example. In a final step, the compound (11) is obtained by coupling between O-(tert-butyldimethylsilyl)hydroxylamine, for example, and the compound (10) under conventional peptide coupling conditions, using, for example, 1-(3-dimethylaminopropyl)-3-ethylcarbodiimide hydrochloride, hydroxybenzotriazole, or TBTU as coupling agents, and triethylamine or diisopropylethylamine as base, in a solvent such as dichloromethane or dimethylformamide. The deprotection of the silylated hydroxamic acid intermediately formed is carried out in situ or by washing with an acidic aqueous solution, so as to give the compound (11).

An alternative synthesis pathway for the compounds with R1 representing a —(CO)—R₄ radical is described in scheme 4.

[chemical structure]

After deprotection of the amine function of the compound (17) according to conventional conditions for hydrogenation in the presence of palladium-on-carbon in a solvent such as ethanol for example, the compound (22) is obtained. The compound (23) is obtained by reacting with an acyl chloride, R₄COCl, in the presence of a base such as triethylamine. When R₂ is a lower alkyl radical, an N-alkylation of the carbamate is then carried out by reacting with an alkyl halide in the presence of a base such as, potassium carbonate in a solvent such as DMF, so as to give the derivative (24). The compound (25) is prepared via a saponification reaction in the presence of a base such as lithium hydroxide in the presence of water and of tetrahydrofuran, for example. Coupling between O-allylhydroxylamine hydrochloride, for example, and the derivative (25) makes it possible to obtain the compound (26) under conventional peptide coupling conditions. For this, use is made, for example, of 1-(3-dimethylaminopropyl)-3-ethylcarbodiimide hydrochloride, hydroxybenzotriazole, or TBTU as coupling agents, and triethylamine or diisopropylethylamine as base. The reaction is carried out in a solvent such as dichloromethane or dimethylformamide.

After deprotection of the amine function of the compound (26) according to conventional methods, the compound (27) is obtained. It is condensed with sulfonyl chloride (15) so as to give the compound (28). In a final step, the compound (29) is obtained by deprotecting the hydroxylamine function of the compound (28) according to conventional methods such as, for example, treatment with tetrakis(triphenylphosphine)palladium(0) and potassium carbonate in methanol.

In one embodiment, the compounds having the structure of formula (I) are those for which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical;
n can take the values of 0, 1, or 2; and,
salts of said compounds including their addition salts with a pharmaceutically acceptable acid, and their addition salts with a pharmaceutically acceptable base; and,
enantiomers of said compounds.

In another embodiment, the compounds having the structure of formula (I) are those for which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a —C(O)—R₄ radical, or a —SO₂—R₄ radical, wherein R₄ is as defined below;
R₂ is a hydrogen atom or a lower alkyl radical;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical;
n can take the values of 1 or 2; and,
salts of said compounds including their addition salts with a pharmaceutically acceptable acid, and their addition salts with a pharmaceutically acceptable base; and,
enantiomers of said compounds.

In other embodiments, the compounds having the structure of formula (I) are those for which:
R₁ is an alkyl radical, a substituted alkyl radical, an aralkyl radical, a substituted aralkyl radical, a —C(O)—R₄ radical, or a —SO₂—R₄ radical, wherein R₄ is as defined below;
R₂ is a hydrogen atom;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical;
n takes the value of 1; and,
salts of said compounds including their addition salts with a pharmaceutically acceptable acid, and their addition salts with a pharmaceutically acceptable base; and, enantiomers of said compounds.
In one aspect the compounds having the structure of formula (I) are those for which:
R₁ is an alkyl radical, a substituted alkyl radical, an aralkyl radical, a substituted aralkyl radical, a —C(O)—R₄ radical, or a —SO₂—R₄ radical, wherein R₄ is as defined below;
R₂ is a hydrogen atom;
R₃ is a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical;
n takes the value of 1; and,
salts of said compounds including their addition salts with a pharmaceutically acceptable acid, and their addition salts with a pharmaceutically acceptable base; and,
enantiomers of said compounds.

In another aspect, the compounds having the structure of formula (I) are those for which:
R₁ is an alkyl radical, a substituted alkyl radical, an aralkyl radical, a substituted aralkyl radical, a —C(O)—R₄ radical, or a —SO₂—R₄ radical, wherein R₄ is as defined below;
R₂ is a hydrogen atom;
R₃ is a heteroaryl radical or a substituted heteroaryl radical;
R₄ is an alkyl radical, a substituted alkyl radical, an aryl radical, a substituted aryl radical, an aralkyl radical, or a substituted aralkyl radical;
n takes the value of 1; and,
salts of said compounds including their addition salts with a pharmaceutically acceptable acid, and their addition salts with a pharmaceutically acceptable base; and,
enantiomers of said compounds.

Provided herein are compounds exhibiting a very good TACE-inhibiting activity. In particular, they inhibit the TACE enzyme selectively compared with other ADAMs and MMPs. This TACE enzyme-inhibiting activity is measured in an enzymatic assay and quantified via the measurement of an IC₅₀ (inhibitory concentration necessary to obtain about 50% inhibition of the TACE enzyme), as described in Example 28. The compounds disclosed herein have an IC₅₀ for TACE less than or equal to about 10 μM and more particularly less than or equal to about 1 μM. Advantageously, the compounds provided herein have an IC₅₀ for TACE less than or equal to about 0.5 μM.

Advantageously, these compounds are also very selective for TACE compared with the other ADAMs and MMPs (assay described in Example 29): the inhibitory activity is at least about 10 times greater for TACE than for other ADAMs and MMPs (i.e. the IC₅₀ value for TACE is at least about 10 times smaller than that for other ADAMs and MMPs), and more advantageously at least about 100 times greater.

TACE (TNFα-converting enzyme) catalyses the formation of soluble TNF-alpha from the precursor protein (transmembrane TNFα) bound to the membranes of certain cells. TNFα is a pro-inflammatory cytokine which is known to play a role in many pathological conditions with an inflammatory nature.

Provided herein are methods for using at least one compound having the structure of formula (I) as defined above, for the treatment of pathological conditions and disorders linked to TNFα release. A TACE enzyme inhibitor having the structure of formula (I) decreases TNFα production. As a result, it is of use for the treatment of pathological conditions linked to TNFα release. Such pathological conditions include but are not limited to inflammatory skin diseases, for example acne, psoriasis, and atopic dermatitis.

In one embodiment, provided herein is a method of using at least one compound having the structure of formula (I) as defined above, for preparing a pharmaceutical or cosmetic composition in which said compound has TACE enzyme-inhibiting activity.

It is therefore directed toward the use of at least one compound having the structure of formula (I) as defined above, for the treatment of pathological conditions or disorders which are improved by inhibiting the TACE enzyme.

In another embodiment, provided herein is a method of therapeutic (human or animal) or cosmetic treatment, which consists essentially of or comprises the administration or the application of a pharmaceutical or cosmetic composition comprising a compound having the structure of formula (I) as a TACE inhibitor and, consequently, as an inhibitor of soluble TNFα production.

Provided herein are methods of using at least one compound having the structure of formula (I) as defined above, for the treatment of pathological conditions or disorders linked to TNFα production.

In one embodiment, provided herein are methods of using a compound having the structure of formula (I) as defined above, for preparing a medicament intended for the treatment of pathological conditions for which reducing TNFαproduction would be of great interest. In another embodiment, provided herein are methods of treating inflammatory diseases.

Indeed, the compounds provided herein are particularly suitable for the treatment and prevention (including substantial inhibition) of disorders/diseases such as the inflammatory diseases listed hereinafter, but are not limited thereto, such as septic shock, hemodynamic shock, malaria, inflammatory bowel disease (IBD) such as Crohn's disease and ulcerative colitis, inflammatory bone disease, mycobacterial infection, meningitis, fibrotic disease, cardiac disease, atherosclerosis, obesity, ischemic attack, transplant rejection, cancer, disease involving angiogenesis phenomena, autoimmune disease, osteoarthritis, rheumatoid arthritis, ankylosing spondylitis, juvenile chronic arthritis, multiple sclerosis, HIV, non-insulin-dependent diabetes mellitus, allergic disease, asthma, and chronic obstructive pulmonary disease (COPD).

The compounds provided herein are also particularly suitable for treating inflammatory skin diseases, such as, acne, psoriasis, atopic dermatitis, and psoriatic arthritis.

These molecules are also potential active ingredients for the treatment of neurological pathological conditions with an inflammatory nature, for which reducing TNFα production would be of great interest. These pathological conditions listed hereinafter in a nonlimiting manner are, for example, Alzheimer's disease, Parkinson's disease, parkinsonian disorder, amyotrophic lateral sclerosis, autoimmune disease of the nervous system, autonomic disease of the nervous system, dorsal pain, cerebral edema, cerebrovascular disorder, dementia, nervous system nerve fiber demyelinating autoimmune disease, diabetic neuropathies, encephalitis, encephalomyelitis, epilepsy, chronic fatigue syndrome, giant cell arteritis, Guillain-Barrésyndrome, headache, multiple sclerosis, neuralgia, peripheral nervous system disease, polyneuropathy, polyradiculoneuropathy, radiculopathy, respiratory paralysis, spinal cord disease, Tourette's syndrome, central nervous system vasculitis, Huntington's disease, and stroke.

Provided herein are methods of using a compound having the structure of formula (I) as defined above, for preparing a medicament intended for the treatment of pathological conditions with an inflammatory nature, in which TNFα is involved.

Also provided herein are methods of using a compound having the structure of formula (I) as defined above, for preparing a medicament intended for the treatment of inflammatory skin diseases, such as acne, psoriasis, atopic dermatitis, and psoriatic arthritis.

In one aspect, provided herein are compositions comprising a compound having the structure of formula (I), a salt thereof, or an enantiomer thereof and a carrier. Compositions provided herein include pharmaceutical compositions. In another aspect, the pharmaceutical compositions, intended in particular for the treatment of the above-mentioned conditions, comprise, in a pharmaceutically-acceptable carrier, which is compatible with the method of administration selected for this composition, at least one compound having the structure of formula (I). This compound can also be in one of its enantiomeric forms or in the form of one of its pharmaceutically-acceptable salts.

In one embodiment, an effective amount of a compound provided herein is administered to a subject in need thereof. In another embodiment, a pharmaceutical composition comprising a therapeutically-effective amount of a compound provided herein, a salt thereof, or an enantiomer thereof, and a pharmaceutically-acceptable carrier is administered to a subject in need thereof. The subject is a mammalian subject. The mammalian subject is a human. In one embodiment, the subject in need thereof is a human patient afflicted with acne.

The therapeutically-effective amount of the compound, a salt thereof, or an enantiomer thereof, is effective to treat an inflammatory disease. In one embodiment, the disease is an inflammatory skin disease, such as acne, psoriasis, atopic dermatitis, and psoriatic arthritis. In another embodiment, the inflammatory skin disease is acne.

The methods disclosed herein comprise administering to a subject in need thereof with a compound or composition disclosed herein. A subject in need thereof may have an inflammatory disease or condition mentioned above.

It has been reported by Kang et al. that transcription factors, nuclear factor-κB (NF-κB) and activator protein-1 (AP-1), were activated in acne lesions with consequent elevated expression of their target gene products, inflammatory cytokines and matrix-degrading metalloproteinases (MMPs) (Kang et al., American Journal of Pathology, Vol. 166 (6): 1691-1699, 2005). These elevated genes are molecular mediators of inflammation and collagen degradation in acne lesions in vivo. Id. The compositions provided herein are used to treat acne by inhibiting inflammatory cytokines and matrix-degrading metalloproteinases. The compounds and compositions inhibit the molecular mediators of inflammation and collagen degradation in acne lesions.

In one aspect, effective amounts of the compounds and compositions can be applied to the skin of a subject in need thereof. The skin of the subject has acne lesions. In another aspect, the skin is a skin sample, such as skin biopsy, from a human patient, and the skin sample comprises acne lesions. In other aspects, the skin can be on a mammalian body, and the mammalian body is that of a human subject.

Provided herein are methods of using the compounds and compositions to inhibit inflammatory the activity and/or production of cytokines and matrix-degrading metalloproteinases associated with acne. In one aspect, provided herein are methods of using the compounds and compositions to inhibit the activation of transcription factors, such as NF-κB and AP-1, in acne lesions. NF-κB up regulates proinflammatory cytokine genes, such as TNFα, IL-1β, IL-8, and IL-10, in acne lesions. Id. AP-1 regulates several MMPs, including but not limited to MMP-1 (collagenase-1), MMP-3 (stromelysin 1), MMP-8 (collagenase 2), MMP-9 (collagenase 4), and MMP-13 (collagenase 3). An increase in MMP activity in skin increases procollagen I and III mRNA level and procollagen I protein level. In another aspect, provided herein are methods of using the compounds and compositions to inhibit the production of cytokines, such as TNFα, IL-1β, IL-8, and IL-10, and MMPs, such as MMP-1, MMP-3, MMP-8, MMP-9, and MMP-13, and procollagens, such as procollagen I and procollagen III, in acne lesion. In one embodiment, the compounds and compositions inhibit the mRNA levels and protein levels of proinflammatory cytokines, MMPs, and procollagens.

In other embodiments, the compounds and compositions provided herein inhibit the inflammation induced by P. acnes. P. acnes induces secretion of cytokines. The methods of using the compounds and compositions provided herein include inhibiting the production of cytokines, such as TNFα and IL-6, induced by P. acnes.

MMP-12 is a matrix metalloproteinase that degrades elastin. Elastin is a protein found in the skin and tissue of the body. Elastin helps to keep skin flexible, so that it returns to its original position, when poked or pinched. Elastin declines as a person ages. MMP-12 is not the only MMP involved in breaking down elastin. Others include, but are not limited to MMP-2 and MMP-9. Provided herein are methods of using the compounds and compositions disclosed herein to inhibit the degradation of elastin. The methods provided herein involve using effective amounts of the compounds and compositions to inhibit MMP-2, MMP-9, and MMP-12.

In one embodiment, the cytokines and MMPs to be inhibited with the compounds and compositions disclosed herein are present in a biological sample, such as a sample comprising cells or a tissue. The cells can be skin cells, and the tissue can be skin. The cells can also be obtained from tissues or subjects diagnosed with an inflammatory disease or condition. In another embodiment, the cells can be in the tissue of the body of a subject diagnosed with an inflammatory disease, such as an inflammatory skin disease. The methods disclosed herein comprise inhibiting one or more cytokines and/or MMPs in a biological sample or in a subject in need thereof.

As used herein the term “about” means approximately or in the region of, such that the values encompassed by the number are related to the significant digits in the number.

As used herein the terms “from” and “between” in the description of values or ranges include the upper and lower limits.

Several examples of preparation of active compounds having formula (I), and also of the results of biological activity of such compounds, will now be given by way of illustration and without being in any way limiting in nature.

### EXAMPLES

The compounds having the structure of formula (I) are characterized by proton NMR analysis on a Bruker Avance 400 MHz instrument.

### Example 1

### 3-[(4-but-2-ynyloxybenzenesulfonyl)methylamino]-N-hydroxy-2-(4-methanesulfonylpiperazin-1-yl)propionamide

### 1-1: Dimethyl 2-(4-tert-butoxycarbonylpiperazin-1-yl)malonate

19.5 g (141 mmol) of potassium carbonate and then 19.5 ml (134 mmol) of dimethyl bromomalonate are added to a solution of 25 g (134 mmol) of tert-butyl piperazine-1-carboxylate in 300 ml of acetonitrile. The reaction medium is stirred at ambient temperature for 24 h and then filtered in order to remove the insoluble salts, and concentrated under vacuum. The crude residue obtained is purified by chromatography on silica gel, elution being carried out with a 70/30 heptane/ethyl acetate mixture. 41 g (97%) of dimethyl 2-(4-tert-butoxycarbonylpiperazin-1-yl)malonate are obtained in the form of a light oil.

### 1-2: Dimethyl 2-(4-tert-butoxycarbonylpiperazin-1-yl)-2-(1,3-dioxo-1,3-dihydroisoindol-2-ylmethyl)malonate

3.5 g (87 mmol) of sodium hydride are added portionwise to a solution of 25 g (87 mmol) of dimethyl 2-(4-tert-butoxycarbonylpiperazin-1-yl)malonate in 250 ml of tetrahydrofuran cooled to 2° C. The reaction medium is stirred at ambient temperature for 30 minutes and then brought back to 2° C., before adding, dropwise, 21 g (87 mmol) of 2-bromomethylisoindole-1,3-dione in 200 ml of tetrahydrofuran. The reaction medium is stirred at ambient temperature for 20 h, treated by adding 500 ml of water and then extracted with ethyl acetate. The organic phase is dried over magnesium sulfate, filtered, and concentrated under vacuum.

The crude product obtained is purified by chromatography on silica gel, elution being carried out with a 70/30 heptane/ethyl acetate mixture. 27.5 g (73%) of dimethyl 2-(4-tert-butoxycarbonylpiperazin-1-yl)-2-(1,3-dioxo-1,3-dihydroisoindol-2-ylmethyl)malonate are obtained in the form of a white solid.

### 1-3: Dimethyl 2-aminomethyl-2-(4-tert-butoxycarbonylpiperazin-1-yl)malonate

A solution of 2.9 ml (64 mmol) of hydrazine hydrate in 8 ml of methanol is added to a solution of 27.5 g (58 mmol) of dimethyl 2-(4-tert-butoxycarbonylpiperazin-1-yl)-2-(1,3-dioxo-1,3-dihydroisoindol-2-ylmethyl)malonate in 300 ml of methanol cooled beforehand to −5° C. The reaction medium is stirred at from −5° C. to ambient temperature over the course of 3 h. After evaporation and addition of 300 ml of water, the reaction medium is extracted with ethyl acetate. The organic phases are washed with a saturated aqueous solution of sodium hydrogen carbonate, dried over magnesium sulfate, filtered, and evaporated. The residue obtained is purified by chromatography on silica gel, elution being carried out with an 8/2 heptane/ethyl acetate mixture and then an increase in polarity up to a 90/10 ethyl acetate/methanol mixture. 10 g (50%) of dimethyl 2-aminomethyl-2-(4-tert-butoxycarbonylpiperazin-1-yl)malonate are thus obtained in the form of a light oil.
1-4: Dimethyl 2-(4-tert-butoxycarbonylpiperazin-1-yl)-2-[(4-but-2-ynyloxybenzenesulfonylamino)methyl]malonate

1.1 ml (8 mmol) of triethylamine and then 1.8 ml (7 mmol) of 4-but-2 ynyloxybenzenesulfonyl chloride are added to a solution of 2.5 g (7 mmol) of dimethyl 2-aminomethyl-2-(4-tert-butoxycarbonylpiperazin-1-yl)malonate in 30 ml of dichloromethane. The reaction medium is stirred at ambient temperature for 2 hours and then concentrated under vacuum. The crude product obtained is purified by chromatography on silica gel, elution being carried out with a 70/30 heptane/ethyl acetate mixture. 2.1 g (51%) of dimethyl 2-(4-tert-butoxycarbonylpiperazin-1-yl)-2-[(4-but-2-ynyloxybenzenesulfonylamino)methyl]malonate are obtained in the form of a white solid.

### 1-5: Dimethyl 2-[(4-but-2-ynyloxybenzenesulfonylamino)methyl]-2-piperazin-1-ylmalonate

2.8 ml of trifluoroacetic acid are added to a solution of 2.1 g (4 mmol) of dimethyl 2-(4-tert-butoxycarbonylpiperazin-1-yl)-2-[(4-but-2-ynyloxybenzenesulfonylamino)methyl]malonate diluted in 30 ml of dichloromethane. After stirring at ambient temperature for 24 h, a saturated aqueous solution of sodium hydrogen carbonate is added to pH=8 and the reaction medium is extracted with dichloromethane. The organic phases are combined, washed with water, dried over magnesium sulfate, and then filtered, and evaporated. 1.7 g (98%) of dimethyl 2-[(4-but-2-ynyloxybenzenesulfonylamino)methyl]-2-piperazin-1-ylmalonate are obtained in the form of a white solid.

### 1-6: Dimethyl 2-[(4-but-2-ynyloxybenzenesulfonylamino)methyl]-2-(4-methanesulfonylpiperazin-1-yl)malonate

0.6 ml (4 mmol) of triethylamine and then 0.3 ml (4 mmol) of methanesulfonyl chloride are added to a solution of 1.6 g (4 mmol) of dimethyl 2-[(4-but-2-ynyloxybenzenesulfonylamino)methyl]-2-piperazin-1-ylmalonate diluted in 30 ml of dichloromethane. The reaction medium is then stirred at ambient temperature for 3 h and then evaporated to dryness. The crude residue is purified by chromatography on silica gel, elution being carried out with a 99/1 dichloromethane/methanol mixture. 1.1 g (58%) of dimethyl 2-[(4-but-2-ynyloxybenzenesulfonylamino)methyl]-2-(4-methanesulfonylpiperazin-1-yl)malonate are obtained in the form of a white solid.

### 1-7: Dimethyl 2-{(4-but-2-ynyloxybenzenesulfonyl)methylamino]methyl}-2-(4-methanesulfonylpiperazin-1-yl)malonate

120 mg (0.9 mmol) of potassium carbonate and then 56 μl (0.9 mmol) of methyl iodide are added to a solution of 400 mg (0.8 mmol) of dimethyl 2-[(4-but-2-ynyloxybenzenesulfonylamino)methyl]-2-(4-methanesulfonylpiperazin-1-yl)malonate in 10 ml of dimethylformamide. The reaction medium is then stirred at ambient temperature for 18 h and then hydrolyzed by adding water and extracted with ethyl acetate. The organic phases are washed with water and then dried over magnesium sulfate, filtered, and concentrated under vacuum. The crude product obtained is purified by chromatography on silica gel, elution being carried out with a 50/50 heptane/ethyl acetate mixture. 410 mg (100%) of dimethyl 2-{[(4-but-2-ynyloxybenzenesulfonyl)methylamino]methyl}-2-(4-methanesulfonylpiperazin-1-yl)malonate are obtained in the form of a white solid.

### 1-8: 3-[(4-But-2-ynyloxybenzenesulfonyl)methylamino]-2-(4-methanesulfonylpiperazin-1-yl)propanoic acid

1.7 ml (1.7 mmol) of an aqueous solution of sodium hydroxide having a concentration of 1M are added to a solution of 270 mg (0.5 mmol) of dimethyl 2-{[(4-but-2-ynyloxybenzenesulfonyl)methylamino]methyl}-2-(4-methanesulfonylpiperazin-1-yl)malonate in 7 ml of tetrahydrofuran and 2 ml of methanol. The reaction medium is stirred at 40° C. for 15 h and then brought back to pH=6 by adding an aqueous solution of hydrochloric acid having a concentration of 1M. After evaporation of the solvents under vacuum, the product precipitates. The residue obtained is taken up in 5 ml of water and stirred for 30 min until precipitation occurs. The product is filtered off, rinsed with water and then dried under vacuum. 200 mg (87%) of 3-[(4-but-2-ynyloxybenzenesulfonyl)methylamino]-2-(4-methanesulfonylpiperazin-1-yl)propanoic acid are obtained in the form of a white solid.

### 1-9: 3-[(4-But-2-ynyloxybenzenesulfonyl)methylamino]-N-hydroxy-2-(4-methanesulfonylpiperazin-1-yl)propionamide

63 mg (0.5 mmol) of 1-hydroxybenzotriazole and then 88 mg (0.5 mmol) of 1-ethyl-3-(3-dimethylaminopropyl)carbodiimide hydrochloride are added to a solution of 200 mg (0.4 mmol) of 3-[(4-but-2-ynyloxybenzenesulfonyl)methylamino]-2-(4-methanesulfonylpiperazin-1-yl)propanoic acid in 6 ml of dimethylformamide. The reaction medium is stirred for 10 min at ambient temperature and then 68 mg (0.5 mmol) of O-tert-butyldimethysilylhydroxylamine are added. The reaction medium is then stirred at ambient temperature for 24 h, hydrolyzed by adding 2 ml of a 5% aqueous citric acid solution, and stirred for a further 30 minutes. After extraction with ethyl acetate, the organic phase is washed with water, dried over magnesium sulfate, filtered, and concentrated. The crude residue is purified by chromatography on silica gel, elution being carried out with a 95/5 dichloromethane/methanol mixture. 100 mg (50%) of 3-[(4-but-2-ynyloxybenzenesulfonyl)methylamino]-N-hydroxy-2-(4-methanesulfonylpiperazin-1-yl)propionamide are obtained in the form of a white solid with a melting point of 86° C.

¹H NMR (δ, DMSO): 1.91 (s, 3H); 2.63-2.68 (m, 2H); 2.72 (s, 3H); 2.72-2.75 (m, 2H); 2.92 (s, 3H); 3.05-3.15 (m, 5H); 3.30-3.38 (m, 2H); 4.93 (s, 2H); 7.24 (d, J=6.8 Hz, 2H); 7.79 (d, J=6.8 Hz, 2H); 9.06 (s, 1H); 10.77 (s, 1H).

### Example 2

### (S)-3-(4-but-2-ynyloxybenzenesulfonylamino)-N-hydroxy-2-(4-methanesulfonylpiperazin-1-yl)propionamide

### 2.1: Sodium salt of 4-but-2-ynyloxybenzenesulfonic acid

50 g (370 mmol) of 1-bromo-2-butyne are added to a solution of 43 g (185 mmol) of commercial sodium salt of 4-hydroxybenzenesulfonic acid and of 185 ml (185 mmol) of an aqueous solution of sodium hydroxide having a concentration of 1M, in 800 ml of isopropanol. The reaction medium is heated at 70° C. for 18 h.

After evaporation of the isopropanol, the product obtained is filtered, rinsed with isopropanol and with diethyl ether and then dried under vacuum. 46 g (100%) of the sodium salt of 4-but-2-ynyloxybenzenesulfonic acid are obtained in the form of a white solid.

### 2.2: 4-But-2-ynyloxybenzenesulfonyl chloride

30 g (107 mmol) of the sodium salt of 4-but-2-ynyloxybenzenesulfonic acid in 120 ml of dimethylformamide are added dropwise to a solution of 28 ml (321 mmol) of oxalyl chloride in 120 ml of dichloromethane, cooled beforehand to −10° C., and then the reaction medium is stirred at ambient temperature for 18 h. 800 ml of ice are added, and the medium is extracted with ethyl acetate. The organic phases are combined, washed with water, dried over magnesium sulfate, filtered, and concentrated under vacuum. 22 g (84%) of 4-but-2-ynyloxybenzenesulfonyl chloride are obtained in the form of a beige solid.

### 2.3: N, N-bis(2-Chloroethyl)methanesulfonamide

8.6 ml (62 mmol) of triethylamine are added to a solution of 5 g (28 mmol) of bis(2-chloroethyl)amine hydrochloride in 60 ml of dichloromethane. The triethylammonium chloride salts precipitate and are filtered off. 2.4 ml (31 mmol) of methylsulfonyl chloride are then added to the filtrate obtained, and the reaction medium is stirred at ambient temperature for 3 h. After the addition of water, the product is extracted with dichloromethane. The organic phase is washed with water, dried over magnesium sulfate, filtered, and concentrated. 5.8 g (94%) of N,N-bis(2-chloroethyl)methanesulfonamide are obtained in the form of a beige solid.

### 2.4: Methyl (S)-3-tert-butoxycarbonylamino-2-(4-methanesulfonylpiperazin-1-yl)propanoate

In a Schlenk tube, a solution of 5 g (20 mmol) of methyl (S)-2-amino-3-tert-butoxycarbonylaminopropanoate hydrochloride and 4.3 g (20 mmol) of N,N-bis(2-chloroethyl)methanesulfonamide in 65 ml of N,N-diisopropylethylamine is heated at 127° C. with vigorous stirring for 18 h. After the addition of water, the product is extracted with ethyl acetate. The organic phases are combined, washed with water, dried over magnesium sulfate, filtered, and concentrated under vacuum. The crude product obtained is purified by chromatography on silica gel, elution being carried out with a 50/50 heptane/ethyl acetate mixture. 3.3 g (46%) of methyl (S)-3-tert-butoxycarbonylamino-2-(4-methanesulfonylpiperazin-1-yl)propanoate are obtained in the form of a white solid.

### 2.5: Methyl (S)-3-amino-2-(4-methanesulfonylpiperazin-1-yl)propanoate hydrochloride

15 ml of a solution of hydrochloric acid in isopropanol, having a concentration of 5-6N are added dropwise to a solution of 2.7 g (7.4 mmol) of methyl (S)-3-tert-butoxycarbonylamino-2-(4-methanesulfonylpiperazin-1-yl)propanoate in 30 ml of methanol. The reaction medium is stirred at 40° C. for 2 h, concentrated under vacuum, and then taken up in 20 ml of methanol and 150 ml of diethyl ether. The product precipitates, and is filtered off under vacuum, rinsed with diethyl ether and then dried under vacuum. 2.3 g (100%) of methyl (S)-3-amino-2-(4-methanesulfonylpiperazin-1-yl)propanoate hydrochloride are obtained in the form of a white solid.

### 2.6: Methyl (S)-3-(4-but-2-ynyloxybenzenesulfonylamino)-2-(4-methanesulfonylpiperazin-1-yl)propanoate

0.3 ml (2 mmol) of triethylamine and 270 mg (1 mmol) of 4-but-2-ynyloxybenzenesulfonyl chloride (prepared as described in 2.2) are added to a solution of 300 mg (1 mmol) of methyl (S)-3-amino-2-(4-methanesulfonylpiperazin-1-yl)propanoate hydrochloride (prepared as described in 2.5) in 8 ml of dichloromethane. After stirring at ambient temperature for 18 h, water is added, and the reaction medium is extracted with dichloromethane. The organic phases are washed with water, dried over magnesium sulfate, filtered, and concentrated.

The crude product obtained is purified by chromatography on silica gel, elution being carried out with a 50/50 heptane/ethyl acetate mixture. 400 mg (85%) of methyl (S)-3-(4-but-2-ynyloxybenzenesulfonylamino)-2-(4-methanesulfonylpiperazin-1-yl)propanoate are obtained in the form of a white solid.

### 2.7: (S)-3-(4-But-2-ynyloxybenzenesulfonylamino)-2-(4-methanesulfonylpiperazin-1-yl)propanoic acid

1.3 ml (1.3 mmol) of an aqueous solution of lithium hydroxide having a concentration of 1M are added to a solution of 400 mg (0.8 mmol) of methyl (S)-3-(4-but-2-ynyloxybenzenesulfonylamino)-2-(4-methanesulfonylpiperazin-1-yl)propanoate diluted in 10 ml of tetrahydrofuran cooled beforehand to 0° C. The reaction medium is stirred at ambient temperature for 20 h. After evaporation to dryness, 1.5 ml of an aqueous solution of acetic acid having a concentration of 1M are added so as to obtain a pH=6. The product precipitates, and is filtered off, rinsed with water, and then with diethyl ether and dried under vacuum. 340 mg (89%) of (S)-3-(4-but-2-ynyloxybenzenesulfonylamino)-2-(4-methanesulfonylpiperazin-1-yl)propanoic acid are obtained in the form of a white solid.

### 2.8: (S)-3-(4-But-2-ynyloxybenzenesulfonylamino)-N-hydroxy-2-(4-methanesulfonylpiperazin-1-yl)propionamide

120 mg (0.9 mmol) of 1-hydroxybenzotriazole and 170 mg (0.9 mmol) of 1-ethyl-3-(3-dimethylaminopropyl)carbodiimide hydrochloride are added to a solution of 340 mg (0.7 mmol) of (S)-3-(4-but-2-ynyloxybenzenesulfonylamino)-2-(4-methanesulfonylpiperazin-1-yl)propanoic acid in 8 ml of dimethylformamide. The reaction medium is stirred for 30 min, and then 120 mg (0.8 mmol) of O-tert-butyldimethysilylhydroxylamine in 3 ml of dimethylformamide are added. The reaction medium is then stirred at ambient temperature for 20 h, and then hydrolyzed with 2 ml of water and 2 ml of a 5% aqueous solution of citric acid. After stirring for 30 min, a saturated aqueous solution of sodium hydrogen carbonate is added to pH=8, and then the reaction medium is extracted with ethyl acetate. The organic phase is dried over magnesium sulfate, filtered, and concentrated. The residue is taken up in dichloromethane, filtered, and then dried under vacuum. 80 mg (23%) of (S)-3-(4-but-2-ynyloxybenzenesulfonylamino)-N-hydroxy-2-(4-methanesulfonylpiperazin-1-yl)propionamide are obtained in the form of a white solid with a melting point of 150° C.

¹H NMR (δ, DMSO): 1.86 (s, 3H); 2.55 (m, 4H); 2.83 (s, 3H); 2.85-2.88 (m, 1H); 2.97-3.00 (m, 3H); 3.00-3.06 (m, 2H); 3.10-3.12 (t, J=4.8 Hz, 1H); 4.86 (s, 2H); 7.15 (d, J=9.2 Hz, 2H); 7.51 (s, 1H); 7.75 (d, J=9.2 Hz, 2H); 8.94 (s, 1H); 10.6 (s, 1H).

### Example 3

### (S)-3-(4-benzyloxy-benzenesulfonylamino)-N-hydroxy-2-(4-methanesulfonylpiperazin-1-yl)propionamide

### 3.1: N, N-bis-(2-Chloroethyl)methanesulfonamide

14.3 ml (185 mmol) of methanesulfonyl chloride are added slowly to a solution of 15 g (84 mmol) of commercial bis(2-chloroethylamine) hydrochloride and 26 ml (185 mmol) of triethylamine in 200 ml of dichloromethane and 70 ml of tetrahydrofuran previously stirred for 15 min and then filtered in order to remove the triethylammonium chloride. The reaction medium is then stirred at ambient temperature for 18 h, extracted with dichloromethane, and washed with water. The organic phase is dried over magnesium sulfate, filtered, and evaporated. The residue obtained is washed with diisopropyl ether, filtered, and then dried under vacuum. 15.3 g (82%) of N,N-bis-(2-chloroethyl)methanesulfonamide are obtained in the form of a solid.

### 3.2: Methyl (S)-3-tert-butoxycarbonylamino-2-(4-methanesulfonylpiperazin-1-yl)propanoate

A solution of 9.6 g (44 mmol) of N,N-bis-(2-chloroethyl)methanesulfonamide and 11.1 g (44 mmol) of methyl 2-amino-3-tert-butoxypropanoate hydrochloride in 90 ml of diisopropylethylamine is heated at 127° C. for 18 h. The reaction medium is evaporated to dryness. 31 g of crude residue are obtained and purified by chromatography on silica gel, elution being carried out with a 9/1 heptane/ethyl acetate mixture and then an increase in polarity up to 4/6. 5.5 g (35%) of methyl (S)-3-tert-butoxycarbonylamino-2-(4-methanesulfonylpiperazin-1-yl)propanoate are obtained.

### 3.3: Methyl (S)-3-amino-2-(4-methanesulfonylpiperazin-1-yl)propanoate dihydrochloride

A solution of 4 g (11 mmol) of methyl (S)-3-tert-butoxycarbonylamino-2-(4-methanesulfonylpiperazin-1-yl)propanoate (prepared as described in Example 2.4) in 40 ml of methanol and 20 ml of a solution of hydrochloric acid in isopropanol, having a concentration of 5 or 6M, is stirred at 40° C. for 18 h and then concentrated under vacuum. The residue obtained is taken up in 200 ml of diethyl ether, filtered, and then dried under vacuum. 3.5 g (94%) of methyl (S)-3-amino-2-(4-methanesulfonylpiperazin-1-yl)propanoate dihydrochloride are obtained in the form of a beige solid.

### 3.4: Sodium salt of 4-benzyloxybenzenesulfonic acid

64 ml (539 mmol) of benzyl bromide are added to a solution of 50 g (215 mmol) of the sodium salt of 4-hydroxybenzenesulfonic acid dihydrate in 700 ml of isopropanol and 250 ml (250 mmol) of an aqueous solution of sodium hydroxide having a concentration of 1M. The reaction medium is heated at 70° C. for 20 h. After concentration of the isopropanol under vacuum, the product precipitates and is filtered off. 61 g (100%) of the sodium salt of 4-benzyloxybenzenesulfonic acid are obtained in the form of a white solid.

### 3.5: 4-Benzyloxybenzenesulfonyl chloride

A solution of 55 ml (639 mmol) of oxalyl chloride in 250 ml of dichloromethane is added dropwise to a solution of 61 g (213 mmol) of the sodium salt of 4-benzyloxybenzenesulfonic acid in 200 ml of dimethylformamide, while maintaining the temperature between −20° C. and −10° C. After addition, the reaction medium is slowly brought back to ambient temperature and then stirred for 18 h, poured onto ice and extracted with ethyl acetate. The organic phase is washed with water and with a saturated aqueous solution of sodium chloride and concentrated under vacuum. 54 g (89%) of 4-benzyloxybenzenesulfonyl chloride are obtained in the form of a white solid.

### 3.6: Methyl (S)-3-(4-benzyloxybenzenesulfonylamino)-2-(4-methanesulfonylpiperazin-1-yl)propanoate

1.1 ml (7.8 mmol) of triethylamine and then 730 mg (2.6 mmol) of 4-benzyloxybenzenesulfonyl chloride in 8 ml of dichloromethane are added to a solution of 800 mg (2.4 mmol) of methyl (S)-3-amino-2-(4-methanesulfonylpiperazin-1-yl)propanoate dihydrochloride in 20 ml of dichloromethane, and the reaction medium is stirred at ambient temperature for 3 h. After the addition of water, the product is extracted with dichloromethane. The organic phase is washed with water, dried over magnesium sulfate, filtered, and concentrated.

The residue obtained is purified by chromatography on silica gel, elution being carried out with an 8/2 heptane/ethyl acetate mixture. 0.9 g (75%) of methyl (S)-3-(4-benzyloxybenzenesulfonylamino)-2-(4-methanesulfonylpiperazin-1-yl)propanoate are obtained in the form of a white solid.

### 3.7: (S)-3-(4-Benzyloxybenzenesulfonylamino)-2-(4-methanesulfonylpiperazin-1-yl)propanoic acid

2.6 ml (2.6 mmol) of an aqueous solution of lithium hydroxide having a concentration of 1M are added to a solution of 900 mg (1.8 mmol) of methyl (S)-3-(4-benzyloxybenzenesulfonylamino)-2-(4-methanesulfonylpiperazin-1-yl)propanoate in 20 ml of tetrahydrofuran and 0.5 ml of water. The reaction medium is stirred at ambient temperature for 18 h and then the THF is evaporated off under vacuum. 2.8 ml of an aqueous solution of acetic acid having of concentration of 1M and then 30 ml of water are added, and the product precipitates. The suspension is stirred for 30 min at 100° C. and then brought back to ambient temperature, filtered, and dried under vacuum. 750 mg (86%) of (S)-3-(4-benzyloxybenzenesulfonylamino)-2-(4-methanesulfonylpiperazin-1-yl)propanoic acid are obtained in the form of a white solid.

### 3.8: (S)-3-(4-Benzyloxybenzenesulfonylamino)-N-hydroxy-2-(4-methanesulfonyl-piperazin-1-yl)propionamide

224 mg (1.7 mmol) of 1-hydroxybenzotriazole and 318 mg (1.7 mmol) of 1-ethyl-3-(3-dimethylaminopropyl)carbodiimide hydrochloride are added successively to 750 mg (1.5 mmol) of (S)-3-(4-benzyloxybenzenesulfonylamino)-2-(4-methanesulfonylpiperazin-1-yl)propanoic acid in 20 ml of dimethylformamide. After stirring at ambient temperature for 20 min, a solution of 244 mg (1.7 mmol) of O-tert-butyldimethylsilylhydroxylamine in 3 ml of dimethylformamide is added. The reaction medium is then stirred at ambient temperature for 18 h and then 2 ml of a saturated aqueous solution of sodium hydrogen carbonate and finally 2 ml of water are added. After extraction with ethyl acetate, the organic phase is washed with a saturated aqueous solution of sodium hydrogen carbonate, dried over magnesium sulfate, filtered, and concentrated. The crude residue obtained is taken up in 15 ml of ethyl acetate, heated to 70° C. and then brought back to ambient temperature, filtered, and dried under vacuum.

300 mg (34%) of (S)-3-(4-benzyloxybenzenesulfonylamino)-N-hydroxy-2-(4-methanesulfonylpiperazin-1-yl)propionamide are obtained in the form of a white solid having a melting point of 165° C.

¹H NMR (δ, DMSO): 2.40-2.50 (m, 2H); 2.50-2.60 (m, 2H); 2.84 (s, 3H), 3.00-3.05 (m, 4H); 3.06-3.09 (m, 2H); 3.34 (s, 1H); 5.19 (s, 2H); 7.19 (d, J=8.4 Hz, 2H); 7.30-7.34 (m, 1H); 7.35-7.47 (m, 5H); 7.73 (d, J=8.4 Hz, 2H); 8.93 (s, 1H); 10.65 (s, 1H).

### Example 4

### (S)-3-[(4-Benzyloxybenzenesulfonyl)methylamino]-N-hydroxy-2-(4-methanesulfonylpiperazin-1-yl)propionamide

### 4.1: Methyl (S)-3-[(4-benzyloxybenzenesulfonyl)methylamino]-2-(4-methanesulfonylpiperazin-1-yl)propanoate

300 mg (1.9 mmol) of potassium carbonate and then 0.2 ml (3.1 mmol) of methyl iodide are added to a solution of 800 mg (1.6 mmol) of methyl (S)-3-(4-benzyloxybenzenesulfonylamino)-2-(4-methanesulfonylpiperazin-1-yl)propanoate (prepared as described in 3.6) in 15 ml of dimethylformamide. The reaction medium is then stirred at ambient temperature for 20 h, hydrolyzed, and then diluted with ethyl acetate. The product is extracted with ethyl acetate. The organic phases are washed with water, dried over magnesium sulfate, and filtered.

The filtrate is concentrated under vacuum, to give 820 mg (100%) of methyl (S)-3-(4-benzyloxybenzenesulfonylamino)-2-(4-methanesulfonylpiperazin-1-yl)propanoate in the form of a white solid.

### 4.2: (S)-3-[(4-Benzyloxybenzenesulfonyl)methylamino]-2-(4-methanesulfonylpiperazin-1-yl)propanoic acid

In a manner analogous to Example 3.7, using 820 mg (1.6 mmol) of methyl (S)-3-[(4-benzyloxybenzenesulfonyl)methylamino]-2-(4-methanesulfonylpiperazin-1-yl)propanoate, 720 mg (90%) of (S)-3-[(4-benzyloxybenzenesulfonyl)methylamino]-2-(4-methanesulfonylpiperazin-1-yl)propanoic acid are obtained in the form of a white solid.

### 4.3: (S)-3-[(4-Benzyloxybenzenesulfonyl)methylamino]-N-hydroxy-2-(4-methanesulfonylpiperazin-1-yl)propionamide

In a manner analogous to Example 3.8, using 720 mg (1.4 mmol) of (S)-3-[(4-benzyloxybenzenesulfonyl)methylamino]-2-(4-methanesulfonylpiperazin-1-yl)propanoic acid, 360 mg (49%) of (S)-3-[(4-benzyloxybenzenesulfonyl)methylamino]-N-hydroxy-2-(4-methanesulfonylpiperazin-1-yl)propionamide are obtained in the form of a white solid with a melting point of 110° C.

¹H NMR (δ, DMSO): 2.58-2.63 (m, 2H); 2.65 (s, 3H); 2.67-2.73 (m, 2H); 2.86 (s, 3H); 2.98-3.05 (m, 4H); 3.05-3.09 (m, 1H); 3.24-3.25 (m, 1H); 3.28-3.31 (m, 1H); 5.21 (s, 2H); 7.24 (d, J=8.9 Hz, 2H); 7.34-7.44 (m, 3H); 7.48 (d, J=7.2 Hz, 2H); 7.72 (d, J=8.9 Hz, 2H); 8.99 (s, 1H); 10.69 (s, 1H).

### Example 5

### (S)—N-Hydroxy-2-(4-methanesulfonylpiperazin-1-yl)-3-[4-(2-methylquinolin-4-ylmethoxy)benzenesulfonylamino]propionamide

### 5.1: Methyl (S)-3-(4-hydroxybenzenesulfonylamino)-2-(4-methanesulfonylpiperazin-1-yl)propanoate

A solution of 2.0 g (3.9 mmol) of methyl (S)-3-(4-benzyloxybenzenesulfonylamino)-2-(4-methanesulfonylpiperazin-1-yl)propanoate (prepared as described in Example 3.6) in 60 ml of ethanol, 30 ml of dioxane and 0.5 ml of glacial acetic acid is degassed under a nitrogen stream and then 200 mg (10% by weight) of palladium-on-carbon at 10% in suspension in 3 ml of dioxane are added. The reaction medium is placed under a hydrogen atmosphere and stirred at ambient temperature for 18 h. After filtration through celite, the filtrate is hydrolyzed and then the product is extracted with ethyl acetate. The organic phase is washed with water and then with a saturated aqueous solution of sodium chloride, dried over magnesium sulfate, filtered, and concentrated under vacuum. 1.65 g (100%) of methyl (S)-3-(4-hydroxybenzenesulfonylamino)-2-(4-methanesulfonylpiperazin-1-yl)propanoate are obtained in the form of a white solid.

### 5.2: Methyl (S)-2-(4-methanesulfonylpiperazin-1-yl)-3-[4-(2-methylquinolin-4-ylmethoxy)benzenesulfonylamino]propanoate

280 mg (0.85 mmol) of cesium carbonate followed by 160 mg (0.85 mmol) of 4-chloromethyl-2-methylquinoline and by 15 mg of potassium iodide are added to a solution of 300 mg (0.71 mmol) of methyl (S)-3-(4-hydroxybenzenesulfonylamino)-2-(4-methanesulfonylpiperazin-1-yl)propanoate in 10 ml of acetone. The reaction medium is stirred at ambient temperature for 18 h, filtered, and concentrated under vacuum. The crude product is purified by chromatography on silica gel, elution being carried out with a 40/60 heptane/ethyl acetate mixture. 130 mg (32%) of methyl (S)-2-(4-methanesulfonylpiperazin-1-yl)-3-[4-(2-methylquinolin-4-ylmethoxy)benzenesulfonylamino]propanoate are obtained in the form of a white solid.

### 5.3: (S)-2-(4-Methanesulfonylpiperazin-1-yl)-3-[4-(2-methylquinolin-4-ylmethoxy)benzenesulfonylamino]propanoic acid

In a manner analogous to Example 3.7, using 130 mg (0.2 mmol) of methyl (S)-2-(4-methanesulfonyl-piperidin-1-yl)-3-[4-(2-methylquinolin-4-ylmethoxy)benzenesulfonylamino]propanoate, 120 mg (99%) of (S)-2-(4-methanesulfonylpiperidin-1-yl)-3-[4-(2-methylquinolin-4-ylmethoxy)benzenesulfonylamino]propanoic acid are obtained in the form of a white solid.

### 5.4: (S)—N-Hydroxy-2-(4-methanesulfonylpiperazin-1-yl)-3-[4-(2-methylquinolin-4-ylmethoxy)benzenesulfonylamino]propionamide

In a manner analogous to Example 3.8, using 123 mg (0.2 mmol) of (S)-2-(4-methanesulfonylpiperazin-1-yl)-3-[4-(2-methylquinolin-4-ylmethoxy)benzenesulfonylamino]propanoic acid, 90 mg (69%) of (S)—N-hydroxy-2-(4-methanesulfonylpiperazin-1-yl)-3-[4-(2-methylquinolin-4-ylmethoxy)benzenesulfonyl-amino]propionamide are obtained in the form of a solid with a melting point of 185° C.

¹H NMR (δ, DMSO): 2.54-2.60 (m, 4H); 2.72 (s, 3H); 2.88 (s, 3H), 2.88-2.93 (m, 1H); 3.01-3.05 (m, 1H); 3.06-3.12 (m, 4H); 3.13-3.16 (t, J=7 Hz, 1H); 5.76 (s, 2H); 7.38 (d, J=8 Hz, 2H); 7.57 (s, 1H); 7.61-7.66 (m, 2H); 7.78-7.85 (m, 3H); 8.02 (d, J=8.2 Hz, 1H); 8.15 (d, J=8.2 Hz, 1H); 8.98 (s, 1H); 10.71 (s, 1H).

### Example 6

### (S)—N-Hydroxy-2-(4-methanesulfonylpiperazin-1-yl)-3-[4-(naphthalen-1-ylmethoxy)benzenesulfonylamino]propionamide

### 6.1: Methyl (S)-2-(4-methanesulfonylpiperazin-1-yl)-3-[4-(2-methylquinolin-4-ylmethoxy)benzenesulfonylamino]propanoate

In a manner analogous to Example 5.2, using 160 mg (0.9 mmol) of 4-chloromethyl-2-methylquinoline and 300 mg (0.7 mmol) of methyl (S)-3-(4-hydroxy-benzenesulfonylamino)-2-(4-methanesulfonylpiperazin-1-yl)propanoate (prepared as described in 5.1), 130 mg (32%) of methyl (S)-2-(4-methanesulfonylpiperazin-1-yl)-3-[4-(2-methylquinolin-4-ylmethoxy)benzenesulfonylamino]propanoate are obtained in the form of a white solid.

### 6.2: (S)-2-(4-Methanesulfonylpiperazin-1-yl)-3-[4-(naphthalen-1-ylmethoxy)benzenesulfonylamino]propanoic acid

In a manner analogous to Example 3.7, using 240 mg (0.6 mmol) of methyl (S)-2-(4-methanesulfonylpiperazin-1-yl)-3-[4-(naphthalen-1-ylmethoxy)benzenesulfonylamino]propanoate, 210 mg (91%) of (S)-2-(4-methanesulfonylpiperazin-1-yl)-3-[4-(naphthalen-1-ylmethoxy)benzenesulfonylamino]-propanoic acid are obtained in the form of a white solid.

### 6.3: (S)—N-Hydroxy-2-(4-methanesulfonylpiperazin-1-yl)-3-[4-(naphthalen-1-ylmethoxy)benzenesulfonylamino]propionamide

In a manner analogous to Example 3.8, using 210 mg (0.4 mmol) of (S)-2-(4-methanesulfonylpiperazin-1-yl)-3-[4-(naphthalen-1-ylmethoxy)benzenesulfonylamino]propanoic acid, 70 mg (33%) of (S)—N-hydroxy-2-(4-methanesulfonylpiperazin-1-yl)-3-[4-(naphthalen-1-ylmethoxy)benzene-sulfonylamino]propionamide are obtained in the form of a beige solid with a melting point of 148° C.

¹H NMR (δ, DMSO): 2.45 (m, 2H); 2.60 (m, 2H); 2.85 (s, 3H); 2.90-3.05 (m, 4H); 3.06-3.15 (m, 2H); 3.35 (s, 1H); 5.66 (s, 2H); 7.30 (d, J=8.4 Hz, 2H); 7.50-7.60 (m, 4H); 7.70 (d, J=6.2 Hz, 1H); 7.77 (d, J=8.2 Hz, 2H); 7.95-8.05 (m, 2H); 8.10 (d, J=6.4 Hz, 1H); 8.94 (s, 1H); 10.70 (s, 1H).

### Example 7

### (S)—N-Hydroxy-2-(4-methanesulfonylpiperazin-1-yl)-3-(4-propoxybenzenesulfonylamino)propionamide

### 7.1: Methyl (S)-2-(4-methanesulfonylpiperazin-1-yl)-3-(4-propoxybenzenesulfonylamino)propanoate

In a manner analogous to Example 5.2, using 0.1 ml (1.3 mmol) of 1-bromopropane and 400 mg (0.95 mmol) of methyl (S)-3-(4-hydroxybenzenesulfonylamino)-2-(4-methanesulfonylpiperazin-1-yl)propanoate (prepared as described in 5.1), 220 mg (50%) of methyl (S)-2-(4-methanesulfonylpiperazin-1-yl)-3-(4-propoxybenzenesulfonylamino)propanoate are obtained in the form of a colorless oil.

### 7.2: (S)-2-(4-Methanesulfonylpiperazin-1-yl)-3-(4-propoxybenzenesulfonylamino)propanoic acid

In a manner analogous to Example 3.7, using 220 mg (0.5 mmol) of methyl (S)-2-(4-methanesulfonylpiperazin-1-yl)-3-(4-propoxybenzenesulfonylamino)propanoate, 190 mg (90%) of (S)-2-(4-methanesulfonylpiperazin-1-yl)-3-(4-propoxybenzenesulfonylamino)propanoic acid are obtained in the form of a white solid.

### 7.3: (S)—N-Hydroxy-2-(4-methanesulfonylpiperazin-1-yl)-3-(4-propoxybenzenesulfonylamino)propionamide

In a manner analogous to Example 3.8, using 190 mg (0.4 mmol) of (S)-2-(4-methanesulfonylpiperazin-1-yl)-3-(4-propoxybenzenesulfonylamino)propanoic acid, 30 mg (16%) of (S)—N-hydroxy-2-(4-methanesulfonylpiperazin-1-yl)-3-(4-propoxybenzenesulfonylamino)propionamide are obtained in the form of a white solid with a melting point of 137° C.

¹H NMR (δ, DMSO): 0.91 (t, J=7.3 Hz, 3H); 1.63-1.73 (m, 2H); 2.45 (m, 2H); 2.55 (m, 2H); 2.77 (s, 3H); 2.82 (m, 1H); 2.83-2.95 (m, 4H); 2.95-3.05 (m, 2H); 3.94 (t, J=6.4 Hz, 2H); 7.03 (d, J=8.7 Hz, 2H); 7.38 (m, 1H); 7.65 (d, J=8.7 Hz, 2H); 8.85 (s, 1H); 10.58 (s, 1H).

### Example 8

### (S)-3-[4-(3-Cyanobenzyloxy)benzenesulfonylamino]-N-hydroxy-2-(4-methanesulfonylpiperazin-1-yl)propionamide

### 8.1: Methyl (S)-3-[4-(3-cyanobenzyloxy)benzenesulfonylamino]-2-(4-methanesulfonylpiperazin-1-yl)propanoate

In a manner analogous to Example 5.2, using 205 mg (1 mmol) of 3-(bromomethyl)benzonitrile and 400 mg (0.95 mmol) of methyl (S)-3-(4-hydroxybenzenesulfonylamino)-2-(4-methanesulfonylpiperazin-1-yl)propanoate (prepared as described in Example 5.1), 295 mg (58%) of methyl (S)-3-[4-(3-cyanobenzyloxy)benzenesulfonylamino]-2-(4-methanesulfonylpiperazin-1-yl)propanoate are obtained in the form of a white solid.

### 8.2: (S)-3-[4-(3-Cyanobenzyloxy)benzenesulfonylamino]-2-(4-methanesulfonylpiperazin-1-yl)propanoic acid

In a manner analogous to Example 3.7, using 295 mg (0.5 mmol) of methyl (S)-3-[4-(3-cyanobenzyloxy)benzenesulfonylamino]-2-(4-methanesulfonylpiperazin-1-yl)propanoate, 270 mg (94%) of (S)-3-[4-(3-cyanobenzyloxy)benzenesulfonylamino]-2-(4-methanesulfonylpiperazin-1-yl)propanoic acid are obtained in the form of a white solid.

### 8.3: (S)-3-[4-(4-Cyanobenzyloxy)benzenesulfonylamino]-N-hydroxy-2-(4-methanesulfonylpiperazin-1-yl)propionamide

In a manner analogous to Example 3.8, using 264 mg (0.5 mmol) of (S)-3-[4-(3-cyanobenzyloxy)benzenesulfonylamino]-2-(4-methanesulfonylpiperazin-1-yl)propanoic acid, 107 mg (40%) of (S)-3-[4-(3-cyanobenzyloxy)benzenesulfonylamino]-N-hydroxy-2-(4-methanesulfonylpiperazin-1-yl)propionamide are obtained in the form of a beige powder with a melting point of 108° C.

¹H NMR (δ, DMSO): 2.55 (m, 4H); 2.84 (s, 3H); 2.95-3.05 (m, 4H); 3.10 (t, J=6.4 Hz, 1H); 3.34 (m; 2H); 5.26 (s, 2H); 7.22 (d, J=8.6 Hz, 2H); 7.50 (s, 1H); 7.64 (t, J=7.6 Hz, 1H); 7.76 (d, J=8.6 Hz, 2H); 7.83 (t, J=8 Hz, 2H); 7.96 (s, 1H); 8.93 (s, 1H); 10.66 (s, 1H).

### Example 9

### (S)-3-[4-(4-Cyanobenzyloxy)benzenesulfonylamino]-N-hydroxy-2-(4-methanesulfonylpiperazin-1-yl)propionamide

### 9.1: Methyl (S)-3-[4-(4-cyanobenzyloxy)benzenesulfonylamino]-2-(4-methanesulfonylpiperazin-1-yl)propanoate

In a manner analogous to Example 5.2, using 400 mg (1 mmol) of methyl (S)-3-(4-hydroxybenzenesulfonylamino)-2-(4-methanesulfonylpiperazin-1-yl)propanoate (prepared as described in 5.1) and 205 mg (1.1 mmol) of 4-(bromomethyl)benzonitrile, 229 mg (45%) of methyl (S)-3-[4-(4-cyanobenzyloxy)benzenesulfonylamino]-2-(4-methanesulfonylpiperazin-1-yl)propanoate are obtained in the form of a white solid.

### 9.2: (S)-3-[4-(4-Cyanobenzyloxy)benzenesulfonylamino]-2-(4-methanesulfonylpiperazin-1-yl)propanoic acid

In a manner analogous to Example 3.7, using 229 mg (0.4 mmol) of methyl (S)-3-[4-(4-cyanobenzyloxy)benzenesulfonylamino]-2-(4-methanesulfonylpiperazin-1-yl)propanoate, 202 mg (91%) (S)-3-[4-(4-cyanobenzyloxy)benzenesulfonylamino]-2-(4-methanesulfonylpiperazin-1-yl)propanoic acid are obtained in the form of a white solid.

### 9.3: (S)-3-[4-(4-Cyanobenzyloxy)benzenesulfonylamino]-N-hydroxy-2-(4-methanesulfonylpiperazin-1-yl)propionamide

In a manner analogous to Example 3.8, using 197 mg (0.4 mmol) of (S)-3-[4-(4-cyanobenzyloxy)benzenesulfonylamino]-2-(4-methanesulfonylpiperazin-1-yl)-propanoic acid, 81 mg (40%) of (S)-3-[4-(4-cyanobenzyloxy)benzenesulfonylamino]-N-hydroxy-2-(4-methanesulfonylpiperazin-1-yl)propionamide are obtained in the form of a beige powder with a melting point of 109° C.

¹H NMR (δ, DMSO): 2.50-2.60 (m, 4H); 2.84 (s, 3H); 2.96-3.01 (m, 4H); 3.09 (t, J=7 Hz, 1H); 3.34 (s, 2H); 5.32 (s, 2H); 7.22 (d, J=8.8 Hz, 2H); 7.50 (m, 1H); 7.66 (d, J=8.1 Hz, 2H); 7.75 (d, J=8.8 Hz, 2H); 7.89 (d, J=8.1 Hz, 2H); 8.93 (s, 1H); 10.66 (s, 1H).

### Example 10

### Benzyl 4-{(S)-1-hydroxycarbamoyl-2-[4-(2-methylquinolin-4-ylmethoxy)benzenesulfonylamino]ethyl}piperazine-1-carboxylate

### 10.1: Benzyl bis(2-chloroethyl)carbamate

13.2 ml (92 mmol) of benzyl chloroformate are added slowly to a solution, cooled to 0° C., of 15 g (84 mmol) of bis(2-chloroethylamine) hydrochloride, 26 ml (185 mmol) of triethylamine in 200 ml of dichloromethane and 70 ml of tetrahydrofuran, stirred beforehand for 15 min and then filtered in order to remove the triethylammonium chloride. The reaction medium is stirred at ambient temperature for 18 h. After the addition of water, the reaction medium is extracted with ethyl acetate. The organic phase is dried over magnesium sulfate, filtered, and evaporated. 20 g of crude residue are obtained and purified by chromatography on silica gel, elution being carried out with an 8/2 heptane/ethyl acetate mixture. 6 g (26%) of benzyl bis(2-chloroethyl)carbamate are thus obtained.

### 10.2: Benzyl 4-((S)-2-tert-butoxycarbonylamino-1-methoxycarbonylethyl)piperazine-1-carboxylate

A solution of 5.5 g (20 mmol) of benzyl bis(2-chloroethyl)carbamate and 5.1 g (20 mmol) of methyl 2-amino-3-tert-butoxypropanoate hydrochloride in 40 ml of diisopropylethylamine is heated at 127° C. for 18 h. After cooling, the reaction medium is evaporated to dryness. 17 g of crude residue are obtained and purified by chromatography on silica gel, elution being carried out with a 9/1 up to 4/6 heptane/ethyl acetate mixture. 1.6 g (19%) of benzyl 4-((S)-2-tert-butoxycarbonylamino-1-methoxycarbonylethyl)piperazine-1-carboxylate.

### 10.3: Benzyl 4-(2-amino-1-methoxycarbonylethyl)piperazine-1-carboxylate dihydrochloride

A solution of 1.45 g (3.4 mmol) of benzyl 4-((S)-2-tert-butoxycarbonylamino-1-methoxycarbonylethyl)piperazine-1-carboxylate in 3.5 ml of a solution of hydrochloric acid in isopropanol, having a concentration 5-6N, and 10 ml of methanol is heated at 40° C. for 3 h and then evaporated. The residue is taken up in diethyl ether and filtered. 1.2 g (90%) of benzyl 4-(2-amino-1-methoxycarbonylethyl)piperazine-1-carboxylate dihydrochloride are obtained in the form of a solid.

### 10.4: Benzyl 4-[(S)-2-(4-hydroxybenzenesulfonylamino)-1-methoxycarbonylethyl]piperazine-1-carboxylate

2.1 ml (15 mmol) of triethylamine and then 920 mg (5 mmol) of 4-hydroxybenzenesulfonyl chloride in 20 ml of dichloromethane are added dropwise to a solution of 1.1 g (3 mmol) of benzyl 4-(2-amino-1-methoxycarbonylethyl)piperazine-1-carboxylate dihydrochloride in 30 ml of dichloromethane, cooled beforehand to 0° C. The reaction medium is then stirred at ambient temperature for 18 h. After the addition of water, the reaction medium is extracted with dichloromethane. The organic phase is with water and then dried over magnesium sulfate, filtered, and concentrated under vacuum. The crude residue obtained is purified by chromatography on silica gel, elution being carried out with a 50/50 heptane/ethyl acetate mixture. 60 mg (46%) of benzyl 4-[2-(4-hydroxybenzenesulfonylamino)-1-methoxycarbonylethyl]piperazine-1-carboxylate are obtained in the form of a white solid.

### 10.5: Benzyl 4-{(S)-1-methoxycarbonyl-2-[4-(2-methylquinolin-4-ylmethoxy)benzenesulfonylamino]ethyl}piperazine-1-carboxylate

In a manner analogous to Example 5.2, using 260 mg (1.4 mmol) of 4-chloromethyl-2-methylquinoline and 600 mg (1.3 mmol) of benzyl 4-[(S)-2-(4-hydroxybenzenesulfonylamino)-1-methoxycarbonylethyl]piperazine-1-carboxylate, 320 mg (40%) of benzyl 4-{(S)-1-methoxycarbonyl-2-[4-(2-methylquinolin-4-ylmethoxy)benzenesulfonylamino]ethyl}piperazine-1-carboxylate are obtained in the form of a white solid.

### 10.6: Benzyl 4-{(S)-1-carboxy-2-[4-(2-methylquinolin-4-ylmethoxybenzenesulfonylamino]ethyl}piperazine-1-carboxylate

In a manner analogous to Example 3.7, using 160 mg (0.25 mmol) of benzyl 4-{(S)-1-methoxycarbonyl-2-[4-(2-methylquinolin-4-ylmethoxy)benzenesulfonylamino]ethyl}piperazine-1-carboxylate, 135 mg (87%) of benzyl 4-{(S)-1-carboxy-2-[4-(2-methylquinolin-4-ylmethoxy-benzenesulfonylamino]ethyl}piperazine-1-carboxylate are obtained in the form of a beige solid.

### 10.7: Benzyl 4-{(S)-1-hydroxycarbamoyl-2-[4-(2-methylquinolin-4-ylmethoxy)benzenesulfonylamino]ethyl}piperazine-1-carboxylate

In a manner analogous to Example 3.8, using 135 mg (0.2 mmol) of benzyl 4-{(S)-1-carboxy-2-[4-(2-methylquinolin-4-ylmethoxybenzenesulfonylamino]ethyl}piperazine-1-carboxylate, 115 mg (82%) of benzyl 4-{(S)-1-hydroxycarbamoyl-2-[4-(2-methylquinolin-4-ylmethoxy)benzenesulfonylamino]ethyl}piperazine-1-carboxylate are obtained in the form of a white solid with a melting point of 162° C.

¹H NMR (δ, DMSO): 2.35-2.45 (m, 4H); 2.70 (s, 3H); 2.80-2.90 (m, 1H); 2.95-3.05 (m, 1H); 3.05-3.10 (m, 1H); 3.25-3.40 (m, 4H); 5.05 (s, 2H); 5.74 (s, 2H); 7.29-7.40 (m, 7H); 7.55 (m, 1H); 7.60-7.70 (m, 2H); 7.79 (d, J=8.8 Hz, 3H); 8.01 (d, J=8 Hz, 1H); 8.14 (d, J=8 Hz, 1H); 8.91 (s, 1H); 10.67 (s, 1H).

### Example 11

### (S)—N-Hydroxy-2-(4-methanesulfonylpiperazin-1-yl)-3-[4-(2-phenylpyridin-4-ylmethoxy)benzenesulfonylamino]propionamide

### 11.1: Methyl (S)-2-(4-methanesulfonylpiperazin-1-yl)-3-[4-(2-phenylpyridin-4-ylmethoxy)benzenesulfonylamino]propanoate

0.23 ml (1.4 mmol) of diethyl azodicarboxylate is added slowly to a solution of 400 mg (0.9 mmol) of methyl (S)-3-(4-hydroxybenzenesulfonylamino)-2-(4-methanesulfonylpiperazin-1-yl)propanoate (prepared as described in Example 5.1), 193 mg (1.0 mmol) of (2-phenylpyridin-4-yl)methanol and 373 mg (1.4 mmol) of triphenylphosphine in 4 ml of tetrahydrofuran. The reaction mixture is stirred for one hour at ambient temperature and then evaporated to dryness. The residue obtained is purified by chromatography on silica gel, elution being carried out with a 60/40 heptane/ethyl acetate mixture. 318 mg (57%) of methyl (S)-2-(4-methanesulfonylpiperazin-1-yl)-3-[4-(2-phenylpyridin-4-ylmethoxy)benzene-sulfonylamino]propanoate are obtained in the form of a white powder.

### 11.2: (S)-2-(4-Methanesulfonylpiperazin-1-yl)-3-[4-(2-phenylpyridin-4-ylmethoxy)benzenesulfonylamino]propanoic acid

In a manner analogous to Example 3.7, using 317 mg (0.5 mmol) of methyl (S)-2-(4-methanesulfonylpiperazin-1-yl)-3-[4-(2-phenylpyridin-4-ylmethoxy)benzenesulfonylamino]propanoate, 298 mg (96%) of (S)-2-(4-methanesulfonylpiperazin-1-yl)-3-[4-(2-phenylpyridin-4-ylmethoxy)benzene-sulfonylamino]propanoic acid are obtained in the form of a white solid.

### 11.3: (S)—N-Hydroxy-2-(4-methanesulfonylpiperazin-1-yl)-3-[4-(2-phenylpyridin-4-ylmethoxy)benzenesulfonylamino]propionamide

In a manner analogous to Example 3.8, using 293 mg (0.5 mmol) of (S)-2-(4-methanesulfonylpiperazin-1-yl)-3-[4-(2-phenylpyridin-4-ylmethoxy)benzenesulfonylamino]propanoic acid, 64 mg (21%) of (S)—N-hydroxy-2-(4-methanesulfonylpiperazin-1-yl)-3-[4-(2-phenylpyridin-4-ylmethoxy)benzenesulfonylamino]propionamide are obtained in the form of a white powder with a melting point of 100° C.

¹H NMR (δ, DMSO): 2.52-2.59 (m, 4H); 2.84 (s, 3H); 2.85-2.90 (m, 1H); 2.90-3.00 (m, 1H); 3.00-3.08 (m, 4H); 3.10 (t, J=7.0 Hz, 1H); 5.35 (s, 2H); 7.26 (d, J=8.9 Hz, 2H); 7.42 (m, 1H); 7.45-7.55 (m, 4H); 7.78 (d, J=8.8 Hz, 2H); 8.03 (s, 1H); 8.10 (d, J=7.0 Hz, 2H); 8.69 (d, J=5.0 Hz, 1H); 8.93 (s, 1H); 10.66 (s, 1H).

### Example 12

### (R)—N-Hydroxy-2-(4-methanesulfonylpiperazin-1-yl)-3-[4-(2-methylquinolin-4-ylmethoxy)benzenesulfonylamino]propionamide

### 12.1: Methyl (R)-3-tert-butoxycarbonylamino-2-(4-methanesulfonylpiperazin-1-yl)propanoate

In a manner analogous to Example 3.2, using 3.8 g (17 mmol) of N,N-bis(2-chloroethyl)methanesulfonamide (prepared as described in 3.1) and 4 g (16 mmol) of commercial methyl (R)-2-amino-3-tert-butoxypropanoate hydrochloride, 2.6 g (46%) of methyl (R)-3-tert-butoxycarbonylamino-2-(4-methanesulfonylpiperazin-1-yl)propanoate are obtained in the form of a light yellow solid.

### 12.2: Methyl (R)-3-amino-2-(4-methanesulfonylpiperazin-1-yl)propanoate dihydrochloride

In a manner analogous to Example 3.3, using 2.5 g (7 mmol) of methyl (R)-3-tert-butoxycarbonylamino-2-(4-methanesulfonylpiperazin-1-yl)propanoate, 2.3 g (100%) of methyl (R)-3-amino-2-(4-methanesulfonylpiperazin-1-yl)propanoate are obtained.

### 12.3: Methyl (R)-3-(4-benzyloxybenzenesulfonylamino)-2-(4-methanesulfonylpiperazin-1-yl)propanoate

In a manner analogous to Example 3.6, using 2.4 g (8.4 mmol) of 4-benzyloxybenzenesulfonyl chloride (prepared as described in Example 3.5) and 2.3 g (7.6 mmol) of methyl (R)-3-amino-2-(4-methanesulfonylpiperazin-1-yl)propanoate dihydrochloride, 3 g (77%) of methyl (R)-3-(4-benzyloxybenzenesulfonylamino)-2-(4-methanesulfonylpiperazin-1-yl)propanoate are obtained in the form of a solid.

### 12.4: Methyl (R)-3-(4-hydroxybenzenesulfonylamino)-2-(4-methanesulfonylpiperazin-1-yl)propanoate

In a manner analogous to Example 5.1, using 3 g (5.9 mmol) of methyl (R)-3-(4-benzyloxybenzenesulfonylamino)-2-(4-methanesulfonylpiperazin-1-yl)propanoate, 2 g (80%) of methyl (R)-3-(4-hydroxybenzenesulfonylamino)-2-(4-methanesulfonylpiperazin-1-yl)propanoate are obtained in the form of a white solid.

### 12.5: Methyl (R)-2-(4-methanesulfonylpiperazin-1-yl)-3-[4-(2-methylquinolin-4-ylmeth-oxy)benzenesulfonylamino]propanoate

In a manner analogous to Example 5.2, using 1 g (2.4 mmol) of methyl (R)-3-(4-hydroxy-benzenesulfonylamino)-2-(4-methanesulfonylpiperazin-1-yl)propanoate and 500 mg (2.6 mmol) of 4-chloromethyl-2-methylquinoline, 740 mg (53%) of methyl (R)-2-(4-methanesulfonylpiperazin-1-yl)-3-[4-(2-methylquinolin-4-ylmethoxy)benzenesulfonylamino]propanoate are obtained in the form of a solid.

### 12.6: (R)-2-(4-Methanesulfonylpiperazin-1-yl)-3-[4-(2-methylquinolin-4-ylmethoxy)benzenesulfonylamino]propanoic acid

In a manner analogous to Example 3.7, using 740 mg (1.3 mmol) of methyl (R)-2-(4-methanesulfonylpiperazin-1-yl)-3-[4-(2-methylquinolin-4-ylmethoxy)benzenesulfonylamino]propanoate, 622 mg (86%) of (R)-2-(4-methanesulfonylpiperazin-1-yl)-3-[4-(2-methylquinolin-4-ylmethoxy)-benzenesulfonylamino]propanoic acid are obtained.

### 12.7: (R)—N-Hydroxy-2-(4-methanesulfonylpiperazin-1-yl)-3-[4-(2-methylquinolin-4-ylmethoxy)benzenesulfonylamino]propionamide

In a manner analogous to Example 3.8, using 620 mg (1.1 mmol) of (R)-2-(4-methanesulfonylpiperazin-1-yl)-3-[4-(2-methylquinolin-4-ylmethoxy)benzenesulfonylamino]propanoic acid, 465 mg (73%) of (R)—N-hydroxy-2-(4-methanesulfonylpiperazin-1-yl)-3-[4-(2-methylquinolin-4-ylmethoxy)benzenesulfonylamino]propionamide are obtained in the form of a white solid.

¹H NMR (δ, DMSO): 2.53 (m, 4H); 2.68 (s, 3H); 2.84 (s, 3H); 2.85 (m, 2H); 2.95-3.05 (m, 4H); 3.10 (m, 1H); 5.72 (s, 2H); 7.35 (d, J=8.8 Hz, 2H); 7.52 (m, 1H); 7.57-7.62 (m, 2H); 7.75-7.82 (m, 3H); 7.98 (d, J=8.4 Hz, 1H); 8.11 (d, J=8.16 Hz, 1H); 8.93 (s, 1H); 10.70 (s, 1H).

### Example 13

### (S)—N-Hydroxy-3-[4-(2-methylquinolin-4-ylmethoxy)benzenesulfonylamino]-2-piperazin-1-ylpropionamide

### 13.1: Benzyl 4-{(S)-1-hydroxycarbamoyl-2-[4-(2-methylquinolin-4-ylmethoxy)benzenesulfonylamino]ethyl}piperazine-1-carboxylate

In a manner analogous to Example 3.8, using 135 mg (0.2 mmol) of benzyl 4-{(S)-1-carboxy-2-[4-(2-methylquinolin-4-ylmethoxybenzenesulfonylamino]ethyl}piperazine-1-carboxylate (prepared as described in 10.6), 115 mg (82%) of benzyl 4-{(S)-1-hydroxycarbamoyl-2-[4-(2-methylquinolin-4-ylmethoxy)benzenesulfonylamino]ethyl}piperazine-1-carboxylate are obtained in the form of a white solid with a melting point of 162° C.

### 13.2: (S)—N-Hydroxy-3-[4-(2-methylquinolin-4-ylmethoxy)benzenesulfonylamino]-2-piperazin-1-ylpropionamide

90 mg (0.15 mmol) of benzyl 4-{(S)-1-hydroxycarbamoyl-2-[4-(2-methylquinolin-4-ylmethoxy)benzenesulfonylamino]ethyl}piperazine-1-carboxylate are placed in solution in 5 ml of dichloromethane and 5 ml of trifluoroacetic acid. The reaction medium is then stirred at ambient temperature for 96 h. After evaporation of the trifluoroacetic acid, the residue is taken up with 5 ml of saturated aqueous solution of sodium hydrogen carbonate and extracted with n-butanol. The organic phase is washed with water and then with a saturated aqueous solution of sodium chloride, dried over magnesium sulfate, filtered, and concentrated under vacuum. The crude product obtained is taken up in a 50/50 heptane/ethyl acetate mixture, stirred for 1 h and then filtered, and dried under vacuum. 50 mg (70%) of (S)—N-hydroxy-3-[4-(2-methylquinolin-4-ylmethoxy)benzenesulfonylamino]-2-piperazin-1-ylpropionamide are obtained in the form of a beige solid with a melting point of 225° C.

¹H NMR (δ, DMSO): 2.35-2.45 (m, 4H); 2.67 (s, 3H); 2.70 (m, 4H); 2.80-3.00 (m, 2H); 3.15 (s, 1H); 5.72 (s, 2H); 7.35 (d, J=8.6 Hz, 2H); 7.70-7.80 (m, 3H); 7.98 (d, J=8.4 Hz, 1H); 8.12 (d, J=8.2 Hz, 1H).

### Example 14

### (S)—N-Hydroxy-2-(4-methanesulfonylpiperazin-1-yl)-3-[4-(2-methylquinolin-4-ylmethoxy)benzenesulfonylamino]propionamide hydrochloride

0.2 ml (1.3 mmol) of a solution of hydrochloric acid in isopropanol having a concentration of 5-6N is added to a solution of 301 mg (0.5 mmol) of (S)—N-hydroxy-2-(4-methanesulfonylpiperazin-1-yl)-3-[4-(2-methylquinolin-4-ylmethoxy)benzenesulfonylamino]propionamide (prepared as described in Example 14) in 10 ml of isopropanol. After stirring at ambient temperature for 1 h, the product precipitates. Through filtration, 927 mg of (S)—N-hydroxy-2-(4-methanesulfonylpiperazin-1-yl)-3-[4-(2-methylquinolin-4-ylmethoxy)benzenesulfonylamino]propionamide hydrochloride are obtained in the form of a white powder. This solid is recrystallized from a 30 ml/5 ml isopropanol/water mixture. 176 mg (52%) of (S)—N-hydroxy-2-(4-methanesulfonylpiperazin-1-yl)-3-[4-(2-methylquinolin-4-ylmethoxy)benzenesulfonylamino]propionamide are obtained in the form of a white powder with a melting point of 209° C.

¹H NMR (δ, DMSO): 2.67 (m, 4H); 2.87 (s, 3H); 2.93 (s, 3H); 3.00-3.15 (m, 4H); 3.22 (m, 1H); 3.35-3.90 (m, 2H); 5.94 (s, 2H); 7.42 (d, J=8.7 Hz, 2H); 7.64 (m, 1H); 7.83-7.90 (m, 3H); 7.98 (m, 1H); 8.05 (m, 1H); 8.30 (d, J=7.6 Hz; 1H); 8.38 (d, J=8.5 Hz, 1H); 9.00 (m, 1H); 10.75 (m, 1H).

### Example 15

### tert-Butyl 3-{4-[(S)-2-hydroxycarbamoyl-2-(4-methanesulfonylpiperazin-1-yl)ethylsulfamoyl]phenoxymethyl}-2-methylindole-1-carboxylate di(trifluoroacetate)

### 15.1: tert-Butyl 3-{4-[(S)-2-(4-methanesulfonylpiperazin-1-yl)-2-methoxycarbonylethylsulfamoyl]-phenoxymethyl}-2-methylindole-1-carboxylate

In a manner analogous to Example 11.1, using 400 mg (0.95 mmol) of methyl (S)-3-(4-hydroxybenzenesulfonylamino)-2-(4-methanesulfonylpiperazin-1-yl)propanoate (prepared as described in Example 5.1) and 248 mg (0.95 mmol) of commercial tert-butyl 3-hydroxymethyl-2-methylindole-1-carboxylate, 326 mg (52%) of tert-butyl 3-{4-[(S)-2-(4-methanesulfonylpiperazin-1-yl)-2-methoxycarbonylethylsulfamoyl]phenoxymethyl}-2-methylindole-1-carboxylate are obtained in the form of a beige powder.

### 15.2: tert-Butyl 3-{4-[(S)-2-carboxy-2-(4-methanesulfonylpiperazin-1-yl)-ethylsulfamoyl]phenoxymethyl}-2-methylindole-1-carboxylate

In a manner analogous to Example 3.7, using 325 mg (0.5 mmol) of tert-butyl 3-{4-[(S)-2-(4-methanesulfonylpiperazin-1-yl)-2-methoxycarbonylethylsulfamoyl]phenoxymethyl}-2-methylindole-1-carboxylate, 179 mg (100%) of tert-butyl 3-{4-[(S)-2-carboxy-2-(4-methanesulfonylpiperazin-1-yl)ethylsulfamoyl]phenoxymethyl}-2-methyl indole-1-carboxylate are obtained in the form of a yellow powder.

### 15.3: tert-Butyl 3-{4-[(S)-2-hydroxycarbamoyl-2-(4-methanesulfonylpiperazin-1-yl)ethylsulfamoyl]phenoxymethyl}-2-methylindole-1-carboxylate di(trifluoroacetate)

45 mg (0.3 mmol) of O-tert-butyldimethylsilylhydroxylamine in solution in 1 ml of dimethylformamide are added to a solution of 179 mg (0.3 mmol) of tert-butyl 3-{4-[(S)-2-carboxy-2-(4-methanesulfonylpiperazin-1-yl)ethylsulfamoyl]phenoxymethyl}-2-methyl-indole-1-carboxylate, 41 mg (0.3 mmol) of 1-hydroxybenzotriazole and 58 mg (0.3 mmol) of 1-ethyl-3-(3-dimethylaminopropyl)carbodiimide hydrochloride in 3 ml of dimethylformamide. The reaction mixture is stirred at ambient temperature for 18 h. After the addition of water and then extraction with ethyl acetate, the organic phases are combined, washed with a saturated solution of sodium hydrogen carbonate and then dried over sodium sulfate, filtered, and evaporated. The residue is purified by preparative HPLC (Gemini C6 phenyl column, 150×3 mm, 3 m; UV detector: 190-420 nm; flow rate: 0.3 ml/mn; solvent A: CH₃CN+0.02% trifluoroacetic acid; solvent B: water+0.02% trifluoroacetic acid).

Gradient:

| | |
|---|---|
| Time | Composition |

| | | |
|---|---|---|
| 0.0 min | A = 5% | B = 95% |
| 20.0 min | A = 98% | B = 2% |
| 30.0 min | A = 98% | B = 2% |

Retention time: 14.6 min, M + 1 = 666.1.

After concentration of the various fractions, 21 mg (10%) of tert-butyl 3-{4-[(S)-2-hydroxycarbamoyl-2-(4-methanesulfonylpiperazin-1-yl)ethylsulfamoyl]phenoxymethyl}-2-methylindole-1-carboxylate di(trifluoroacetate) are obtained.

### Example 16

### (S)—N-Hydroxy-2-(4-methanesulfonylpiperazin-1-yl)-3-[4-(quinolin-4-ylmethoxy)benzenesulfonylamino]propionamide

### 16.1: Methyl (S)-2-(4-methanesulfonylpiperazin-1-yl)-3-[4-(quinolin-4-ylmethoxy)benzenesulfonylamino]propanoate

In a manner analogous to Example 5.2, using 440 mg (2.5 mmol) of 4-chloromethylquinoline and 950 mg (2.2 mmol) of methyl (S)-3-(4-hydroxybenzenesulfonylamino)-2-(4-methanesulfonylpiperazin-1-yl)propanoate (prepared as described in 5.1), 550 mg (43%) of methyl (S)-2-(4-methanesulfonylpiperazin-1-yl)-3-[4-(quinolin-4-ylmethoxy)benzenesulfonylamino]propanoate are obtained in the form of a colorless oil.

### 16.2: (S)-2-(4-Methanesulfonylpiperazin-1-yl)-3-[4-(quinolin-4-ylmethoxy)benzenesulfonylamino]propanoic acid

In a manner analogous to Example 3.7, using 550 mg (1.0 mmol) of methyl (S)-2-(4-methanesulfonylpiperazin-1-yl)-3-[4-(quinolin-4-ylmethoxy)benzenesulfonylamino]propanoate, 450 mg (83%) (S)-2-(4-methanesulfonylpiperazin-1-yl)-3-[4-(quinolin-4-ylmethoxy)benzenesulfonylamino]-propanoic acid are obtained in the form of a white solid.

### 16.3: (S)—N-Hydroxy-2-(4-methanesulfonylpiperazin-1-yl)-3-[4-(quinolin-4-ylmethoxy)benzenesulfonylamino]propionamide

In a manner analogous to Example 3.8, using 450 mg (0.8 mmol) of (S)-2-(4-methanesulfonylpiperazin-1-yl)-3-[4-(quinolin-4-ylmethoxy)benzenesulfonylamino]propanoic acid, 260 mg (56%) of (S)—N-hydroxy-2-(4-methanesulfonylpiperazin-1-yl)-3-[4-(quinolin-4-ylmethoxy)benzenesulfonylamino]propionamide are obtained in the form of a white solid with a melting point of 180° C.

¹H NMR (δ, DMSO): 2.52-2.54 (m, 4H); 2.84 (s, 3H); 2.87 (m, 1H); 2.97 (m, 1H); 2.98-3.05 (m, 4H), 3.11 (t, J=7 Hz, 1H); 5.78 (s, 2H); 7.34 (d, J=8.8 Hz, 2H); 7.52 (m, 1H); 7.66-7.72 (m, 2H); 7.78-7.84 (m, 3H); 8.10 (d, J=8.3 Hz, 1H); 8.19 (d, J=8.2 Hz, 1H); 8.93 (s, 1H); 8.94 (s, 1H); 10.67 (s, 1H).

### Example 17

### (S)-2-(4-Benzylpiperazin-1-yl)-N-hydroxy-3-[4-(2-methylquinolin-4-ylmethoxy)benzenesulfonylamino]propionamide

### 17.1: Sodium salt of 4-(2-methylquinolin-4-ylmethoxy)benzenesulfonic acid

100 g (438 mmol) of 4-chloromethyl-2-methylquinoline hydrochloride are added to a solution of 77 g (395 mmol) of the sodium salt of 4-hydroxybenzenesulfonic acid and of 84 ml (84 mmol) of an aqueous solution of sodium hydroxide, having a concentration of 1M, in 800 ml of isopropanol. The reaction medium is heated at 70° C. for 5 h and then at 40° C. for 18 h.

After evaporation of the isopropanol, the product obtained is filtered, rinsed with isopropanol and with diethyl ether and then dried under vacuum. 114 g (75%) of the sodium salt of 4-(2-methylquinolin-4-ylmethoxy)benzenesulfonic acid are obtained in the form of a white solid.

### 17.2: 4-(2-Methylquinolin-4-ylmethoxy)benzenesulfonyl chloride

76 g (216 mmol) of the sodium salt of 4-(2-methylquinolin-4-ylmethoxy)benzenesulfonic acid in 500 ml of dimethylformamide are added dropwise to a solution of 55 ml (649 mmol) of oxalyl chloride in 100 ml of dichloromethane, cooled beforehand to −10° C. After the addition, the reaction medium is stirred at ambient temperature for 18 h. The reaction medium is then poured into 1 I of ice and then extracted with ethyl acetate. The organic phases are combined, washed with water and then with a saturated aqueous solution of sodium chloride, dried over magnesium sulfate, filtered, and concentrated under vacuum. 77 g (92%) of 4-(2-methylquinolin-4-ylmethoxy)benzenesulfonyl chloride hydrochloride are obtained in the form of a beige solid.

### 17.3: Benzylbis(2-chloroethyl)amine

21 g (152 mmol) of potassium carbonate and then 8 ml (67 mmol) of benzyl bromide are added to a solution of 10 g (56 mmol) of bis(2-chloroethyl)amine hydrochloride in 130 ml of acetonitrile, and then the reaction medium is heated at 60° C. for 24 h. After filtration, the filtrate is concentrated under vacuum. The crude residue is purified by chromatography on silica gel, elution being carried out with a 90/10 heptane/ethyl acetate mixture, to give 8.5 g (65%) of benzylbis(2-chloroethyl)amine.

### 17.4: Methyl (S)-2-(4-benzylpiperazin-1-yl)-3-tert-butoxycarbonylaminopropanoate

A solution of 5.9 g (23 mmol) of commercial methyl (S)-2-amino-3-tert-butoxycarbonylaminopropanoate hydrochloride and of 9.6 g (23 mmol) of benzylbis(2-chloroethyl)amine in 50 ml of N,N-diisopropylethylamine is heated at 127° C. for 3 h 30. After evaporation of the N,N-diisopropylethylamine, the reaction medium is hydrolyzed and then extracted with ethyl acetate. The organic phase is washed with an aqueous solution of sodium hydroxide having a concentration of 1N, and with water, and then dried over magnesium sulfate, filtered, and concentrated under vacuum. The crude product obtained is purified by chromatography on silica gel, elution being carried out with a 50/50 heptane/ethyl acetate mixture. 8.9 g (64%) of methyl (S)-2-(4-benzylpiperazin-1-yl)-3-tert-butoxycarbonylaminopropanoate are obtained in the form of a yellow oil.

### 17.5: Methyl (S)-3-amino-2-(4-benzylpiperazin-1-yl)propanoate trihydrochloride

8.9 g (23.5 mmol) of methyl (S)-2-(4-benzylpiperazin-1-yl)-3-tert-butoxycarbonylaminopropanoate are placed in solution in 60 ml of methanol and in 20 ml of isopropanolic hydrochloric acid having a concentration of 5-6N. The reaction medium is stirred at 40° C. for 18 h and then concentrated under vacuum. 9.0 g (100%) of methyl (S)-3-amino-2-(4-benzylpiperazin-1-yl)propanoate trihydrochloride are obtained in the form of a beige solid.

### 17.6: Methyl (S)-2-(4-benzylpiperazin-1-yl)-3-[4-(2-methylquinolin-4-ylmethoxy)benzenesulfonylamino]propanoate

In a manner analogous to Example 3.6, using 1.0 g (2.6 mmol) of methyl (S)-3-amino-2-(4-benzylpiperazin-1-yl)propanoate trihydrochloride and 1.1 g (2.8 mmol) of 4-(2-methylquinolin-4-ylmethoxy)benzenesulfonyl chloride in hydrochloride form, 750 mg (50%) of methyl (S)-2-(4-benzylpiperazin-1-yl)-3-[4-(2-methylquinolin-4-ylmethoxy)benzenesulfonylamino]propanoate are obtained in the form a beige solid.

### 17.7: (S)-2-(4-Benzylpiperazin-1-yl)-3-[4-(2-methylquinolin-4-ylmethoxy)benzenesulfonylamino]propanoic acid

In a manner analogous to Example 3.7, using 750 mg (1.3 mmol) of methyl (S)-2-(4-benzylpiperazin-1-yl)-3-[4-(2-methylquinolin-4-ylmethoxy)benzenesulfonylamino]propanoate, 680 mg (93%) of (S)-2-(4-benzylpiperazin-1-yl)-3-[4-(2-methylquinolin-4-ylmethoxy)benzenesulfonylamino]propanoic acid are obtained in the form of a white solid.

### 17.8: (S)-2-(4-Benzylpiperazin-1-yl)-N-hydroxy-3-[4-(2-methylquinolin-4-ylmethoxy)benzenesulfonylamino]propionamide

In a manner analogous to Example 3.8, using 680 mg (1.2 mmol) of (S)-2-(4-benzylpiperazin-1-yl)-3-[4-(2-methylquinolin-4-ylmethoxy)benzenesulfonylamino]propanoic acid, 250 mg (36%) of (S)-2-(4-benzylpiperazin-1-yl)-N-hydroxy-3-[4-(2-methyl quinolin-4-ylmethoxy)benzenesulfonyl-amino]propionamide are obtained in the form of a white solid with a melting point of 188° C.

¹H NMR (δ, DMSO): 2.33 (m, 4H); 2.49 (m, 4H); 2.73 (s, 3H); 2.80-2.90 (m, 1H); 3.00-3.10 (m, 2H); 2.46 (m, 2H); 5.77 (s, 2H); 7.25-7.40 (m, 7H); 7.50 (m, 1H); 7.61-7.67 (m, 2H); 7.78-7.85 (m, 3H); 8.04 (d, J=8 Hz, 1H); 8.17 (d, J=8.2 Hz, 1H); 8.95 (s, 1H); 10.65 (s, 1H).

### Example 18

### (S)-2-[4-(4-Fluorobenzyl)piperazin-1-yl]-N-hydroxy-3-[4-(2-methylquinolin-4-ylmethoxy)benzenesulfonylamino]propionamide

### 18.1: Bis(2-chloroethyl) (4-fluorobenzyl)amine

In a manner analogous to Example 17.3, using 5 g (28 mmol) of bis(2-chloroethyl)amine hydrochloride and 3.8 ml (31 mmol) of 1-bromomethyl-4-fluorobenzene, 6.9 g (98%) of bis(2-chloroethyl)(4-fluorobenzyl)amine are obtained.

### 18.2: Methyl (S)-3-tert-butoxycarbonylamino-2-[4-(4-fluorobenzyl)piperazin-1-yl]propanoate

In a manner analogous to Example 17.4, using 7.1 g (28 mmol) of methyl (S)-2-amino-3-tert-butoxycarbonylaminopropanoate hydrochloride and 6.9 g (28 mmol) of bis(2-chloroethyl)(4-fluorobenzyl)amine, 5.3 g (48%) of methyl (S)-3-tert-butoxycarbonylamino-2-[4-(4-fluorobenzyl)piperazin-1-yl]propanoate are obtained in the form of an oil.

### 18.3: Methyl (S)-3-amino-2-[4-(4-fluorobenzyl)piperazin-1-yl]propanoate trihydrochloride

In a manner analogous to Example 17.5, using 5.3 g (13.4 mmol) of methyl (S)-3-tert-butoxycarbonylamino-2-[4-(4-fluorobenzyl)piperazin-1-yl]propanoate, 5.4 g (100%) of methyl (S)-3-amino-2-[4-(4-fluorobenzyl)piperazin-1-yl]propanoate trihydrochloride are obtained in the form of a beige solid.

### 18.4: Methyl (S)-2-[4-(4-fluorobenzyl)piperazin-1-yl]-3-[4-(2-methylquinolin-4-ylmethoxy)benzenesulfonylamino]propanoate

In a manner analogous to Example 3.6, using 1.5 g (3.7 mmol) of methyl (S)-3-amino-2-[4-(4-fluorobenzyl)piperazin-1-yl]propanoate trihydrochloride and 1.6 g (4.1 mmol) of 4-(2-methylquinolin-4-ylmethoxy)benzenesulfonyl chloride hydrochloride (prepared as described in 17.2), 1.0 g (46%) of methyl (S)-2-[4-(4-fluorobenzyl)piperazin-1-yl]-3-[4-(2-methylquinolin-4-ylmethoxy)benzenesulfonyl-amino]propanoate is obtained in the form of a white solid.

### 18.5: (S)-2-[4-(4-fluorobenzyl)piperazin-1-yl]-3-[4-(2-methylquinolin-4-ylmethoxy)benzenesulfonylamino]propanoic acid

In a manner analogous to Example 3.7, using 1.1 g (1.7 mmol) of methyl (S)-2-[4-(4-fluorobenzyl)piperazin-1-yl]-3-[4-(2-methylquinolin-4-ylmethoxy)benzenesulfonylamino]propanoate, 1.0 g (100%) of (S)-2-[4-(4-fluorobenzyl)piperazin-1-yl]-3-[4-(2-methylquinolin-4-ylmethoxy)benzene-sulfonylamino]propanoic acid are obtained in the form of a white solid.

### 18.6: (S)-2-[4-(4-Fluorobenzyl)piperazin-1-yl]-N-hydroxy-3-[4-(2-methylquinolin-4-ylmethoxy)benzenesulfonylamino]propionamide

In a manner analogous to Example 3.8, using 990 mg (1.7 mmol) of (S)-2-[4-(4-fluorobenzyl)-piperazin-1-yl]-3-[4-(2-methylquinolin-4-ylmethoxy)benzenesulfonylamino]propanoic acid, 330 mg (33%) of (S)-2-[4-(4-fluorobenzyl)piperazin-1-yl]-N-hydroxy-3-[4-(2-methylquinolin-4-ylmethoxy)benzene-sulfonylamino]propionamide are obtained in the form of a white solid with a melting point of 180° C.

¹H NMR (δ, DMSO): 2.20-2.30 (m, 4H); 2.35-2.45 (m, 4H); 2.66 (s, 3H); 2.72-2.80 (m, 1H); 2.87-3.00 (m, 2H); 3.38 (s, 2H); 5.70 (s, 2H); 7.10 (t, J=8.8 Hz, 2H); 7.26-7.33 (m, 4H); 7.56-7.60 (m, 2H); 7.73-7.78 (m, 3H); 7.97 (d, J=8.4 Hz, 1H); 8.10 (d, J=8.2 Hz, 1H).

### Example 19

### (S)-2-(4-ethyl-piperazin-1-yl)-N-hydroxy-3-[4-(2-methylquinolin-4-yl-methoxy)benzenesulfonylamino]propionamide

### 19.1: bis(2-Chloroethyl)ethylamine

24 ml (330 mmol) of thionyl chloride are added dropwise to a solution of 20 g (150 mmol) of 2-[ethyl(2-hydroxyethyl)amino]ethanol in 200 ml of dichloromethane cooled beforehand to 0° C., and then the reaction medium is stirred at ambient temperature for 20 h. After the addition of a saturated aqueous solution of sodium hydrogen carbonate, the product is extracted with dichloromethane. The organic phase obtained is then washed with water, dried over magnesium sulfate, filtered, and concentrated under vacuum. 19.5 g (76%) of bis(2-chloroethyl)ethylamine are obtained in the form of an oil.

### 19.2: Methyl (S)-3-tert-butoxycarbonylamino-2-(4-ethylpiperazin-1-yl)propanoate

A solution of 5.0 g (19.6 mmol) of commercial methyl (S)-2-amino-3-tert-butoxycarbonylaminopropanoate hydrochloride and 3.3 g (19.6 mmol) of bis(2-chloroethyl)ethylamine in 50 ml of N,N-diisopropylethylamine is heated at 127° C. for 5 h. After evaporation of a maximum amount of diisopropylethylamine, the reaction medium is diluted with ethyl acetate and washed with an aqueous solution of sodium hydroxide having a concentration of 1N. The organic phase obtained is then washed with water, dried over magnesium sulfate, filtered, and concentrated under vacuum. The crude product obtained is purified by chromatography on silica gel, elution being carried out with a 30/70 heptane/ethyl acetate mixture. 2.5 g (40%) of methyl (S)-3-tert-butoxycarbonylamino-2-(4-ethylpiperazin-1-yl)propanoate are obtained in the form of an oil.

### 19.3: Methyl (S)-3-amino-2-(4-ethylpiperazin-1-yl)propanoate trihydrochloride

2.5 g (7.9 mmol) of methyl (S)-3-tert-butoxycarbonylamino-2-(4-ethylpiperazin-1-yl)propanoate are placed in 20 ml of methanol and 10 ml of isopropanolic hydrochloric acid having a concentration of 5-6N. The reaction medium is heated at 40° C. for 3 h and then evaporated to dryness. The residue is taken up in 50 ml of ethanol, stirred for 1 h at ambient temperature and then filtered. 1.4 g (54%) of methyl (S)-3-amino-2-(4-ethylpiperazin-1-yl)propanoate trihydrochloride are obtained in the form of a beige solid.

### 19.4: Methyl (S)-2-(4-ethylpiperazin-1-yl)-3-[4-(2-methylquinolin-4-ylmethoxy)benzenesulfonylamino]propanoate

In a manner analogous to Example 3.6, using 700 mg (2.1 mmol) of methyl (S)-3-amino-2-(4-ethylpiperazin-1-yl)propanoate trihydrochloride and 900 mg (2.3 mmol) of 4-(2-methylquinolin-4-ylmethoxy)benzenesulfonyl chloride hydrochloride (prepared as described in 17.2), 740 mg (67%) of methyl (S)-2-(4-ethylpiperazin-1-yl)-3-[4-(2-methylquinolin-4-ylmethoxy)benzenesulfonylamino]propanoate are obtained in the form of a white solid.

### 19.5: (S)-2-(4-Ethylpiperazin-1-yl)-3-[4-(2-methylquinolin-4-ylmethoxy)benzenesulfonylamino]propanoic acid

In a manner analogous to Example 3.7, using 740 mg (1.4 mmol) of methyl (S)-2-(4-ethylpiperazin-1-yl)-3-[4-(2-methylquinolin-4-ylmethoxy)benzenesulfonylamino]propanoate, 630 mg (87%) of (S)-2-(4-ethylpiperazin-1-yl)-3-[4-(2-methylquinolin-4-ylmethoxy)benzenesulfonylamino]propanoic acid are obtained in the form of a white solid.

### 19.6: (S)-2-(4-Ethylpiperazin-1-yl)-N-hydroxy-3-[4-(2-methylquinolin-4-ylmethoxy)benzenesulfonylamino]propionamide

In a manner analogous to Example 3.8, using 630 mg (1.2 mmol) of (S)-2-(4-ethylpiperazin-1-yl)-3-[4-(2-methylquinolin-4-ylmethoxy)benzenesulfonylamino]propanoic acid, 60 mg (8%) of (S)-2-(4-ethylpiperazin-1-yl)-N-hydroxy-3-[4-(2-methylquinolin-4-ylmethoxy)benzenesulfonylamino]propion-amide are obtained in the form of a white solid with a melting point of 150° C.

¹H NMR (δ, DMSO): 2.49 (s, 3H); 2.55-2.65 (m, 2H); 2.69 (s, 3H); 2.70-2.90 (m, 6H); 2.90-3.00 (m, 2H); 3.13 (t, J=7.3 Hz, 1H); 3.20-3.35 (m, 2H); 3.36 (s, 2H); 5.72 (s, 2H); 7.35 (d, J=8.9 Hz, 2H); 7.58-7.62 (m, 3H); 7.74-7.81 (m, 3H); 7.98 (d, J=7.9 Hz, 1H); 8.12 (d, J=8.3 Hz, 1H); 9.03 (s, 1H), 10.82 (s, 1H).

### Example 20

### (S)—N-Hydroxy-3-[4-(2-methylquinolin-4-ylmethoxy)benzenesulfonylamino]-2-[4-(4-trifluoromethylbenzyl)piperazin-1-yl]propionamide

### 20.1: bis-(2-Chloroethyl) (4-trifluoromethylbenzyl)amine

In a manner analogous to Example 32.3, using 5.0 g (28 mmol) of bis(2-chloroethyl)amine hydrochloride and 7.4 g (31 mmol) of 1-bromomethyl-4-trifluoromethylbenzene, 5 g (59%) of a bis(2-chloroethyl)(4-trifluoromethylbenzyl)amine mixture are obtained in the form of a colorless oil.

### 20.2: Methyl (S)-3-tert-butoxycarbonylamino-2-[4-(4-trifluoromethylbenzyl)piperazin-1-yl]propanoate

A solution of 4.2 g (16.5 mmol) of methyl (S)-2-amino-3-tert-butoxycarbonylaminopropanoate hydrochloride and 4.95 g (16.5 mmol) of bis(2-chloroethyl)(4-trifluoromethylbenzyl)amine in 25 ml of N,N-diisopropylethylamine is heated at 127° C. for 6 h. After evaporation of a maximum amount of diisopropylethylamine, the reaction medium is diluted with ethyl acetate and washed with an aqueous solution of sodium hydroxide having a concentration of 1N. The organic phase obtained is washed with water, dried over magnesium sulfate, filtered, and concentrated under vacuum. The crude residue is purified by chromatography on silica gel, elution being carried out with a 60/40 heptane/ethyl acetate mixture. 4.0 g (55%) of methyl (S)-3-tert-butoxycarbonylamino-2-[4-(4-trifluoromethylbenzyl)piperazin-1-yl]propanoate are obtained in the form of an oil.

### 20.3: Methyl (S)-3-amino-2-[4-(4-trifluoromethylbenzyl)piperazin-1-yl]propanoate trihydrochloride

In a manner analogous to Example 17.5, using 4 g (9.1 mmol) of methyl (S)-3-tert-butoxycarbonylamino-2-[4-(4-trifluoromethylbenzyl)piperazin-1-yl]propanoate, 3.8 g (93%) of methyl (S)-3-amino-2-[4-(4-trifluoromethylbenzyl)piperazin-1-yl]propanoate are obtained in the form of a beige solid.

### 20.4: Methyl (S)-3-[4-(2-methylquinolin-4-ylmethoxy)benzenesulfonylamino]-2-[4-(4-trifluoromethylbenzyl)piperazin-1-yl]propanoate

In a manner analogous to Example 3.6, using 1.0 g (2.2 mmol) of methyl (S)-3-amino-2-[4-(4-trifluoromethylbenzyl)piperazin-1-yl]propanoate trihydrochloride and 1.2 g (3.1 mmol) of 4-(2-methylquinolin-4-ylmethoxy)benzenesulfonyl chloride hydrochloride (prepared as described in Example 17.2), 910 mg (65%) of methyl (S)-3-[4-(2-methylquinolin-4-ylmethoxy)benzenesulfonylamino]-2-[4-(4-trifluoromethylbenzyl)piperazin-1-yl]propanoate are obtained in the form of a white solid.

### 20.5: (S)-3-[4-(2-Methylquinolin-4-ylmethoxy)benzenesulfonylamino]-2-[4-(4-trifluoromethylbenzyl)piperazin-1-yl]propanoic acid

In a manner analogous to Example 3.7, using 910 mg (1.4 mmol) of methyl (S)-3-[4-(2-methylquinolin-4-ylmethoxy)benzenesulfonylamino]-2-[4-(4-trifluoromethylbenzyl)piperazin-1-yl]-propanoate, 790 mg (88%) of (S)-3-[4-(2-methylquinolin-4-ylmethoxy)benzenesulfonylamino]-2-[4-(4-trifluoromethylbenzyl)-piperazin-1-yl]propanoic acid are obtained in the form of a white solid.

### 20.6: (S)—N-Hydroxy-3-[4-(2-methylquinolin-4-ylmethoxy)benzenesulfonylamino]-2-[4-(4-trifluoromethylbenzyl)piperazin-1-yl]propionamide

In a manner analogous to Example 3.8, using 790 mg (1.2 mmol) of (S)-3-[4-(2-methylquinolin-4-ylmethoxy)benzenesulfonylamino]-2-[4-(4-trifluoromethylbenzyl)piperazin-1-yl]propanoic acid, 550 mg (68%) of (S)—N-hydroxy-3-[4-(2-methylquinolin-4-ylmethoxy)benzenesulfonylamino]-2-[4-(4-trifluoro-methylbenzyl)piperazin-1-yl]propionamide are obtained in the form of a white solid with a melting point of 148° C.

¹H NMR (δ, DMSO): 2.21 (m, 4H); 2.38 (m, 4H); 2.58 (s, 3H); 2.69-2.75 (m, 1H); 2.85-2.93 (m, 1H); 2.93-2.98 (m, 1H); 3.42 (s, 2H); 5.63 (s, 2H); 7.25 (d, J=9 Hz, 2H); 7.40 (d, J=8 Hz, 3H); 7.47-7.53 (m, 2H); 7.57 (d, J=8.1 Hz, 2H); 7.65-7.72 (m, 3H); 7.90 (d, J=7.9 Hz, 1H); 8.03 (d, J=7.8 Hz, 1H); 8.83 (s, 1H), 10.56 (s, 1H).

### Example 21

### (S)—N-hydroxy-2-[4-(4-methylbenzyl)piperazin-1-yl]-3-[4-(2-methylquinolin-4-ylmethoxy)benzenesulfonylamino]propionamide

### 21.1: bis(2-Chloroethyl) (4-methylbenzyl)amine

In a manner analogous to Example 17.3, using 5.0 g (28 mmol) of bis(2-chloroethyl)amine hydrochloride and 5.7 g (31 mmol) of 1-bromomethyl-4-methylbenzene, 4.9 g (71%) of bis(2-chloroethyl)(4-methylbenzyl)amine are obtained.

### 21.2: Methyl (S)-3-tert-butoxycarbonylamino-2-[4-(4-methylbenzyl)piperazin-1-yl]propanoate

In a manner analogous to Example 17.4, using 5.1 g (20 mmol) of commercial methyl (S)-2-amino-3-tert-butoxycarbonylaminopropanoate hydrochloride and 4.9 g (20 mmol) of bis(2-chloroethyl)(4-methylbenzyl)amine, 4.1 g (53%) of methyl (S)-3-tert-butoxycarbonylamino-2-[4-(4-methylbenzyl)piperazin-1-yl]propanoate are obtained in the form of an oil.

### 21.3: Methyl (S)-3-amino-2-[4-(4-methylbenzyl)piperazin-1-yl]propanoate trihydrochloride

In a manner analogous to Example 19.3, using 4.1 g (10.5 mmol) of methyl (S)-3-tert-butoxycarbonylamino-2-[4-(4-methylbenzyl)piperazin-1-yl]propanoate, 3.95 g (94%) of methyl (S)-3-amino-2-[4-(4-methylbenzyl)piperazin-1-yl]propanoate trihydrochloride are obtained in the form of a cream solid.

### 21.4: Methyl (S)-3-[4-(2-methylquinolin-4-ylmethoxy)benzenesulfonylamino]-2-[4-(4-methylbenzyl)piperazin-1-yl]propanoate

In a manner analogous to Example 3.6, using 1.0 g (2.5 mmol) of methyl (S)-3-amino-2-[4-(4-methylbenzyl)piperazin-1-yl]propanoate trihydrochloride and 1.3 g (3.5 mmol) of 4-(2-methylquinolin-4-ylmethoxy)benzenesulfonyl chloride hydrochloride (prepared as described in Example 17.2), 950 mg (63%) of methyl (S)-3-[4-(2-methylquinolin-4-ylmethoxy)benzenesulfonylamino]-2-[4-(4-methylbenzyl)piperazin-1-yl]propanoate are obtained in the form of a white solid.

### 21.5: (S)-2-[4-(4-Methylbenzyl)piperazin-1-yl]-3-[4-(2-methylquinolin-4-ylmethoxy)benzenesulfonylamino]propanoic acid

In a manner analogous to Example 3.7, using 950 mg (1.6 mmol) of methyl (S)-2-[4-(4-methylbenzyl)piperazin-1-yl]-3-[4-(2-methylquinolin-4-ylmethoxy)benzenesulfonylamino]propanoate, 880 mg (95%) of (S)-2-[4-(4-methylbenzyl)piperazin-1-yl]-3-[4-(2-methylquinolin-4-ylmethoxy)benzenesulfonylamino]propanoic acid are obtained in the form of a cream solid.

### 21.6: (S)—N-Hydroxy-3-[4-(2-methylquinolin-4-ylmethoxy)benzenesulfonylamino]-2-[4-(4-methylbenzyl)piperazin-1-yl]propionamide

In a manner analogous to Example 3.8, using 880 mg (1.5 mmol) of (S)-3-[4-(2-methylquinolin-4-ylmethoxy)benzenesulfonylamino]-2-[4-(4-methylbenzyl)piperazin-1-yl]propanoic acid, 150 mg (17%) of (S)—N-hydroxy-3-[4-(2-methylquinolin-4-ylmethoxy)benzenesulfonylamino]-2-[4-(4-methylbenzyl)piperazin-1-yl]propionamide are obtained in the form of a white solid with a melting point of 170° C.

¹H NMR (δ, DMSO): 2.25 (m, 4H); 2.25 (s, 3H); 2.43 (m, 4H); 2.67 (s, 3H); 2.80 (m, 1H); 2.95-3.05 (m, 2H); 3.37 (m, 2H); 5.71 (s, 2H); 7.10 (q, J=8 Hz, 4H); 7.33 (d, J=8.9 Hz, 2H); 7.43 (m, 1H); 7.56-7.61 (m, 2H); 7.73-7.79 (m, 3H); 7.98 (d, J=8.3 Hz, 1H); 8.11 (d, J=8.2 Hz, 1H); 8.89 (s, 1H); 10.59 (s, 1H).

### Example 22

### (S)-3-[4-(benzoisoxazol-3 ylmethoxy)benzenesulfonylamino]-N-hydroxy-2-(4-methanesulfonylpiperazin-1-yl)propionamide

### 22.1: Benzoisoxazol-3-ylmethanol

589 mg (3.0 mmol) of ethyl 1.2-benzoisoxazole-3-carboxylate in solution in 10 ml of tetrahydrofuran are added to a suspension of 129 mg (3.5 mmol) of lithium aluminum hydride in 5 ml of tetrahydrofuran. The reaction mixture is stirred for one hour at 60° C. and then treated by adding 2 ml of methanol dropwise, filtered through celite and rinsed with ethyl acetate. The organic phases are combined, dried over sodium sulfate and evaporated. The residue obtained is purified by chromatography on silica gel, elution being carried out with a 60/40 heptane/ethyl acetate mixture. 180 mg (39%) of benzoisoxazol-3-ylmethanol are obtained in the form of a white solid.

### 22.2: Methyl (S)-3-[4-(benzoisoxazol-3-ylmethoxy)benzenesulfonylamino]-2-(4-methanesulfonylpiperazin-1-yl)propanoate

In a manner analogous to Example 11.1, using 494 mg (1.2 mmol) of methyl (S)-3-(4-hydroxy-benzenesulfonylamino)-2-(4-methanesulfonylpiperazin-1-yl)propanoate (prepared as described in 5.1) and 175 mg (1.2 mmol) of benzoisoxazol-3-ylmethanol, 459 mg (71%) of methyl (S)-3-[4-(benzoisoxazol-3-ylmethoxy)benzenesulfonylamino]-2-(4-methanesulfonylpiperazin-1-yl)propanoate are obtained in the form of an oil.

### 22.3: (S)-3-[4-(benzoisoxazol-3-ylmethoxy)benzenesulfonylamino]-2-(4-methanesulfonylpiperazin-1-yl)propanoic acid

In a manner analogous to Example 3.7, using 458 mg (0.8 mmol) of methyl (S)-3-[4-(benzoisoxazol-3-ylmethoxy)benzenesulfonylamino]-2-(4-methanesulfonylpiperazin-1-yl)propanoate, 283 mg (63%) of (S)-3-[4-(benzoisoxazol-3-ylmethoxy)benzenesulfonylamino]-2-(4-methanesulfonylpiperazin-1-yl)propanoic acid are obtained in the form of a white solid.

### 22.4: (S)-3-[4-(benzoisoxazol-3-ylmethoxy)benzenesulfonylamino]-N-hydroxy-2-(4-methanesulfonylpiperazin-1-yl)propionamide

In a manner analogous to Example 3.8, using 283 mg (0.5 mmol) of (S)-3-[4-(benzoisoxazol-3-ylmethoxy)benzenesulfonylamino]-2-(4-ethanesulfonylpiperazin-1-yl)propanoic acid, 231 mg (80%) of (S)-3-[4-(benzoisoxazol-3-ylmethoxy)benzenesulfonylamino]-N-hydroxy-2-(4-methanesulfonylpiperazin-1-yl)propionamide are obtained in the form of a beige solid with a melting point of 107° C.

¹H NMR (δ, DMSO): 2.53-2.55 (m, 4H); 2.88 (s, 3H); 2.90-2.93 (m, 2H); 3.00-3.10 (m, 4H); 3.13 (t, J=6.9 Hz, 1H); 5.77 (s, 2H); 7.35 (d, J=8.8 Hz, 2H); 7.49 (t, J=7.5 Hz, 1H); 7.57 (m, 1H); 7.75 (t, J=7.4 Hz, 1H); 7.78-7.87 (m, 3H); 8.01 (d, J=8 Hz, 1H); 8.96 (m, 1H); 10.67 (m, 1H).

### Example 23

### (S)—N-hydroxy-2-(4-isobutyrylpiperazin-1-yl)-3-[4-(2-methylquinolin-4-ylmethoxy)-benzenesulfonylamino]propionamide

### 23.1: Methyl (S)-3-tert-butoxycarbonylamino-2-piperazin-1-ylpropanoate

2 g (25% by weight) of palladium-on-carbon at 10% are added to a solution of 8 g (21 mmol) of methyl(S)-2-(4-benzylpiperazin-1-yl)-3-tert-butoxycarbonylaminopropanoate (prepared as described in Example 17.4) in 120 ml of ethanol, degassed beforehand under a nitrogen stream. The reaction medium is then placed under a hydrogen atmospheric pressure for 24 h and then filtered through celite and thoroughly rinsed with dichloromethane. After concentration under vacuum, 6.1 g (100%) of methyl (S)-3-tert-butoxycarbonylamino-2-piperazin-1-ylpropanoate are obtained.

### 23.2: Methyl (S)-3-tert-butoxycarbonylamino-2-(4-isobutyrylpiperazin-1-yl)propanoate

1.2 ml (8.3 mmol) of triethylamine and then 0.8 ml (7.6 mmol) of isobutyryl chloride are added to a solution of 2.0 g (6.9 mmol) of methyl (S)-3-tert-butoxycarbonylamino-2-piperazin-1-ylpropanoate in 20 ml of dichloromethane, cooled beforehand to 0° C. After stirring at ambient temperature for 1 h 30, water is added. The reaction medium is extracted with dichloromethane. The organic phase is washed with water, dried over magnesium sulfate, filtered, and concentrated under vacuum. The crude residue obtained is purified by chromatography on silica gel, elution being carried out with a 50/50 heptane/ethyl acetate mixture. 2.0 g (81%) of methyl (S)-3-tert-butoxycarbonylamino-2-(4-isobutyrylpiperazin-1-yl)propanoate are obtained in the form of a colorless oil.

### 23.3: (S)-3-tert-butoxycarbonylamino-2-(4-isobutyrylpiperazin-1-yl)propanoic acid

10 ml (10 mmol) of an aqueous solution of lithium hydroxide having a concentration of 1N are added to a solution of 2.0 g (5.6 mmol) of methyl (S)-3-tert-butoxycarbonylamino-2-(4-isobutyrylpiperazin-1-yl)propanoate in 40 ml of tetrahydrofuran and 8 ml of water, and then the reaction medium is stirred at ambient temperature for 20 h. After the addition of an aqueous solution of acetic acid having a concentration of 1N, the product is extracted with n-butanol. The organic phase is dried over magnesium sulfate, filtered, and concentrated under vacuum, 1.5 g (78%) of (S)-3-tert-butoxycarbonylamino-2-(4-isobutyrylpiperazin-1-yl)propanoic acid are obtained in the form of a white solid.

### 23.4: tert-Butyl [(S)-2-allyloxycarbamoyl-2-(4-isobutyrylpiperazin-1-yl)ethyl]carbamate

1.4 g (4.4 mmol) of O-(benzotriazol-1-yl)-N,N,N′,N′-tetramethyluroniumtetrafluoroborate and then 2.3 ml (13.1 mmol) of diisopropylethylamine are added to a solution of 1.5 g (4.4 mmol) of (S)-3-tert-butoxycarbonylamino-2-(4-isobutyrylpiperazin-1-yl)propanoic acid in 20 ml of dimethylformamide. After stirring at ambient temperature for 15 min, a solution of 500 mg (4.6 mmol) of O-allylhydroxylamine hydrochloride and of 0.8 ml (4.6 mmol) of diisopropylethylamine in 10 ml of dimethylformamide is added. The reaction medium is stirred at ambient temperature for 20 h, hydrolyzed with a saturated aqueous solution of sodium hydrogen carbonate, and then diluted with ethyl acetate. The organic phase is washed with a saturated aqueous solution of sodium chloride, dried over magnesium sulfate, filtered, and concentrated under vacuum, 1.45 g (83%) of tert-butyl [(S)-2-allyloxycarbamoyl-2-(4-isobutyrylpiperazin-1-yl)ethyl]carbamate are obtained in the form of a colorless oil.

### 23.5: (S)—N-allyloxy-3-amino-2-(4-isobutyrylpiperazin-1-yl)propionamide dihydrochloride

In a manner analogous to Example 19.3, using 1.45 g (3.6 mmol) of tert-butyl [(S)-2-allyloxycarbamoyl-2-(4-isobutyrylpiperazin-1-yl)ethyl]carbamate, 1.4 g (100%) of (S)—N-allyloxy-3-amino-2-(4-isobutyrylpiperazin-1-yl)propionamide dihydrochloride are obtained in the form of a white solid.

### 23.6: (S)—N-allyloxy-2-(4-isobutyrylpiperazin-1-yl)-3-[4-(2-methylquinolin-4-ylmethoxy)benzenesulfonylamino]propionamide

1.9 g (5.1 mmol) of 4-(2-methylquinolin-4-ylmethoxy)benzenesulfonyl chloride hydrochloride (prepared as described in 32.2) are added to a solution of 1.3 g (3.6 mmol) of (S)—N-allyloxy-3-amino-2-(4-isobutyrylpiperazin-1-yl)propionamide dihydrochloride, 2.0 ml (14.5 mmol) of triethylamine in 15 ml of dichloromethane and 15 ml of dimethylformamide, cooled beforehand to 0° C. The reaction medium is then stirred at from 0° C. to ambient temperature over the course of 3 h. After the addition of water, the reaction medium is extracted with dichloromethane. The organic phase is washed with a saturated aqueous solution of sodium hydrogen carbonate and with water, dried over magnesium sulfate, filtered, and concentrated.

The crude residue obtained is purified by silica column chromatography, elution being carried out with a 97/3 dichloromethane/methanol mixture. 900 mg (41%) of (S)—N-allyloxy-2-(4-isobutyrylpiperazin-1-yl)-3-[4-(2-methylquinolin-4-ylmethoxy)benzenesulfonylamino]propionamide are obtained in the form of a white solid.

### 23.7: (S)—N-hydroxy-2-(4-isobutyrylpiperazin-1-yl)-3-[4-(2-methylquinolin-4-ylmethoxy)-benzenesulfonylamino]propionamide

33 mg (0.06 mmol) of tetrakis(triphenylphosphine)palladium and then 920 mg (6.6 mmol) of potassium carbamate are added to a solution of 670 mg (1.1 mmol) of (S)—N-allyloxy-2-(4-isobutyrylpiperazin-1-yl)-3-[4-(2-methylquinolin-4-ylmethoxy)benzenesulfonylamino]propionamide in 15 ml of methanol and then the reaction medium is refluxed for 8 h. After the addition of ethyl acetate, the reaction medium is washed with a saturated aqueous solution of sodium hydrogen carbonate. The organic phase is then washed with water, dried over magnesium sulfate, filtered, and concentrated. The crude product is taken up in 6 ml of ethanol and 12 ml of water and then heated at 80° C. until solubilization occurs. After cooling, crystallization is initiated by evaporation of a minimum amount of ethanol. 120 mg of product are obtained by filtration and are purified by preparative thin layer chromatography on silica, elution being carried out with a 97/3 dichloromethane/methanol mixture.

20 mg (3%) of (S)—N-hydroxy-2-(4-isobutyrylpiperazin-1-yl)-3-[4-(2-methylquinolin-4-ylmethoxy)benzenesulfonylamino]propionamide are finally obtained in the form of a beige solid.

¹H NMR (δ, DMSO): 0.84 (s, 3H); 0.85 (s, 3H); 2.30-2.44 (m, 2H); 2.52 (m, 2H); 2.67 (s, 3H); 2.77 (m, 1H); 2.85 (m, 2H); 2.95 (m, 1H); 3.35 (m, 4H); 5.71 (s, 2H); 7.33 (d, J=8.9 Hz, 2H); 7.43 (m, 1H); 7.55-7.62 (m, 2H); 7.72-7.82 (m, 3H); 7.98 (d, J=8.4 Hz, 1H); 8.11 (d, J=8.2 Hz, 1H); 8.96 (m, 1H); 10.67 (m, 1H).

### Example 24

### (S)—N-hydroxy-2-[4-(2-methylpropane-1-sulfonyl)piperazin-1-l]-3-[4-(2-methylquinolin-4-ylmethoxy)benzenesulfonylamino]propionamide

### 24.1: Methyl (S)-3-tert-butoxycarbonylamino-2-[4-(2-methylpropane-1-sulfonyl)piperazin-1-yl]propanoate

479 mg (3.0 mmol) of 2-methylpropane-1-sulfonyl chloride are added to a solution of 800 mg (2.8 mmol) of methyl (S)-3-tert-butoxycarbonylamino-2-piperazin-1-ylpropanoate (prepared as described in Example 23.1) and 775 μl (5.5 mmol) of triethylamine in 8 ml of dichloromethane, cooled beforehand to 0° C. The reaction medium is stirred at ambient temperature for 18 h and then water is added and the medium is extracted with dichloromethane. The organic phase is washed with water, dried over magnesium sulfate, filtered, and concentrated. The residue obtained is purified by chromatography on silica gel, elution being carried out with a 5/5 heptane/ethyl acetate mixture. 785 mg (71%) of methyl (S)-3-tert-butoxycarbonylamino-2-[4-(2-methylpropane-1-sulfonyl)piperazin-1-yl]propanoate are obtained in the form of a colorless oil.

### 24.2: Methyl (S)-3-amino-2-[4-(2-methylpropane-1-sulfonyl)piperazin-1-yl]propanoate dihydrochloride

In a manner analogous to Example 3.3, using 785 mg (1.9 mmol) of methyl (S)-3-tert-butoxycarbonylamino-2-[4-(2-methylpropane-1-sulfonyl)piperazin-1-yl]propanoate, 621 mg (85%) of methyl (S)-3-amino-2-[4-(2-methylpropane-1-sulfonyl)piperazin-1-yl]propanoate dihydrochloride are obtained in the form of a solid.

### 24.3: Methyl (S)-2-[4-(2-methylpropane-1-sulfonyl)piperazin-1-yl]-3-[4-(2-methylquinolin-4-ylmethoxy)benzenesulfonylamino]propanoate

In a manner analogous to Example 3.6, using 621 mg (1.6 mmol) of methyl (S)-3-amino-2-[4-(2-methylpropane-1-sulfonyl)piperazin-1-yl]propanoate dihydrochloride and 876 mg (2.3 mmol) of 4-(2-methylquinolin-4-ylmethoxy)benzenesulfonyl chloride hydrochloride (prepared as described in Example 17.2), 643 mg (64%) of methyl (S)-2-[4-(2-methyl propane-1-sulfonyl)piperazin-1-yl]-3-[4-(2-methylquinolin-4-ylmethoxy)benzenesulfonylamino]propanoate are obtained in the form of an oil.

### 24.4: (S)-2-[4-(2-methylpropane-1-sulfonyl)piperazin-1-yl]-3-[4-(2-methylquinolin-4-ylmethoxy)benzenesulfonylamino]propanoic acid

In a manner analogous to Example 3.7, using 643 mg (1.0 mmol) of methyl (S)-2-[4-(2-methylpropane-1-sulfonyl)piperazin-1-yl]-3-[4-(2-methylquinolin-4-ylmethoxy)benzenesulfonylamino]propanoate, 395 mg (63%) of (S)-2-[4-(2-methylpropane-1-sulfonyl)piperazin-1-yl]-3-[4-(2-methylquinolin-4-ylmethoxy)benzenesulfonylamino]propanoic acid are obtained in the form of a white solid.

### 24.5: (S)—N-hydroxy-2-[4-(2-methylpropane-1-sulfonyl)piperazin-1-yl]-3-[4-(2-methylquinolin-4-ylmethoxy)benzenesulfonylamino]propionamide

In a manner analogous to Example 3.8, using 390 mg (0.6 mmol) of (S)-2-[4-(2-methylpropane-1-sulfonyl)piperazin-1-yl]-3-[4-(2-methylquinolin-4-ylmethoxy)benzenesulfonylamino]propanoic acid, 12 mg (3%) of (S)—N-hydroxy-2-[4-(2-methylpropane-1-sulfonyl)piperazin-1-yl]-3-[4-(2-methylquinolin-4-ylmethoxy)benzenesulfonylamino]propionamide are obtained in the form of a white solid.

¹H NMR (δ, DMSO): 1.01 (d, J=6.7 Hz, 6H); 2.05 (m, 1H); 2.49 (m, 4H); 2.67 (s, 3H); 2.86 (d, J=6.6 Hz, 2H); 3.00-3.10 (m, 6H); 3.31 (m, 1H); 5.71 (s, 2H); 7.34 (d, J=8.9 Hz, 2H); 7.52 (m, 1H); 7.57 (m, 2H); 7.76-7.80 (m, 3H); 7.98 (d, J=8.2 Hz, 1H); 8.10 (m, 1H); 8.93 (s, 1H); 10.66 (s, 1H).

### Example 25

### (S)—N-hydroxy-2-(4-methanesulfonylpiperazin-1-yl)-3-[4-(2-trifluoromethyl-pyrazolo[1,5-a]pyridin-3-ylmethoxy)benzenesulfonylamino]propionamide

### 25.1: Ethyl 2-trifluoromethylpyrazolo[1,5-a]pyridine-3-carboxylate

A solution of 2.1 g (38 mmol) of KOH in 20 ml of water and then 6.7 g (30 mmol) of 1-aminopyridinium iodide are added to a solution of 2.5 g (15 mmol) of ethyl 4,4,4-trifluorobut-2-ynoate in 25 ml of dichloromethane. After stirring at ambient temperature for 5 h, water is added and the reaction medium is extracted with dichloromethane. The organic phase is washed with water, dried over magnesium sulfate. filtered, and concentrated. The residue obtained is purified by chromatography on silica gel, elution being carried out with an 8/2 heptane/ethyl acetate mixture. 2.8 g (73%) of ethyl 2-trifluoromethylpyrazolo[1,5-a]pyridine-3-carboxylate are obtained in the form of a yellow solid.

### 25.2: (2-trifluoromethylpyrazolo[1,5-a]pyridin-3-yl)methanol

A solution of 2.8 g (11 mmol) of ethyl 2-trifluoromethylpyrazolo[1,5-a]pyridine-3-carboxylate in 50 ml of tetrahydrofuran is added dropwise to a suspension of 0.5 g (12 mmol) of lithium aluminum hydride in 45 ml of tetrahydrofuran. The reaction medium is then stirred at 70° C. for 3 h. After dropwise addition of 2.5 ml of methanol and then of 1.8 ml of an aqueous solution of sodium hydroxide having a concentration of 2N, the reaction medium is stirred for 20 min at ambient temperature and then filtered. The filtrate is dried over magnesium sulfate, filtered, and concentrated under vacuum. 2.3 g (100%) of (2-trifluoromethylpyrazolo[1,5-a]pyridin-3-yl)methanol are obtained in the form of a solid.

### 25.3: Methyl (S)-2-(4-methanesulfonylpiperazin-1-yl)-3-[4-(2-trifluoromethylpyrazolo[1,5-a]pyridin-3-ylmethoxy)benzenesulfonylamino]propanoate

In a manner analogous to Example 11.1, using 800 mg (1.9 mmol) of methyl (S)-3-(4-hydroxybenzenesulfonylamino)-2-(4-methanesulfonylpiperazin-1-yl)propanoate (prepared as described in 5.1) and 540 mg (2.5 mmol) of (2-trifluoromethylpyrazolo[1,5-a]pyridin-3-yl)methanol, 380 mg (32%) of methyl (S)-2-(4-methanesulfonylpiperazin-1-yl)-3-[4-(2-trifluoromethylpyrazolo[1,5-a]pyridin-3-yl-methoxy)benzenesulfonylamino]propanoate are obtained in the form of a white solid.

### 25.4: (S)-2-(4-methanesulfonylpiperazin-1-yl)-3-[4-(2-trifluoromethylpyrazolo[1,5-a]pyridin-3-ylmethoxy)benzenesulfonylamino]propanoic acid

In a manner analogous to Example 3.7, using 380 mg (0.6 mmol) of methyl (S)-2-(4-methanesulfonylpiperazin-1-yl)-3-[4-(2-trifluoromethylpyrazolo[1,5-a]pyridin-3-ylmethoxy)benzenesulfonylamino]propanoate, 237 mg (64%) of (S)-2-(4-methanesulfonylpiperazin-1-yl)-3-[4-(2-trifluoromethylpyrazolo[1,5-a]pyridin-3-ylmethoxy)benzenesulfonylamino]propanoic acid are obtained in the form of a white solid.

### 25.5: (S)—N-hydroxy-2-(4-methanesulfonylpiperazin-1-yl)-3-[4-(2-trifluoromethylpyrazolo[1,5-a]pyridin-3-ylmethoxy)benzenesulfonylamino]propionamide

In a manner analogous to Example 3.8, using 230 mg (0.4 mmol) of (S)-2-(4-methanesulfonyl-piperazin-1-yl)-3-[4-(2-trifluoromethylpyrazolo[1,5-a]pyridin-3-ylmethoxy)benzenesulfonylamino]propanoic acid, 9 mg (4%) of (S)—N-hydroxy-2-(4-methanesulfonylpiperazin-1-yl)-3-[4-(2-trifluoromethylpyrazolo[1,5-a]pyridin-3-ylmethoxy)benzenesulfonylamino]propionamide are obtained in the form of a white solid.

¹H NMR (δ, DMSO): 2.51-2.54 (m, 4H); 2.84 (s, 3H); 2.95 (m, 1H); 2.97-3.04 (m, 4H); 3.10 (m, 1H); 3.32 (m, 1H); 5.45 (s, 2H); 7.20-7.25 (m, 3H); 7.49-7.51 (m, 2H); 7.76 (d, J=8.8 Hz, 2H); 8.04 (m, 1H); 8.87 (d, J=7 Hz, 2H); 8.90 (m, 1H).

### Example 26

### (S)—N-hydroxy-3-[4-(2-methylquinolin-4-ylmethoxy)benzenesulfonylamino]-2-[4-(propane-2-sulfonyl)piperazin-1-yl]propionamide

### 26.1: Methyl (S)-3-tert-butoxycarbonylamino-2-[4-(propane-2-sulfonyl)piperazin-1-yl]propanoate

In a manner analogous to Example 20.2, using 800 mg (2.8 mmol) of methyl (S)-3-tert-butoxycarbonylamino-2-piperazin-1-ylpropanoate (prepared as described in example 23.1) and 342 μl (3.1 mmol) of propane-2-sulfonyl chloride, 700 mg (64%) of methyl (S)-3-tert-butoxycarbonylamino-2-[4-(propane-2-sulfonyl)piperazin-1-yl]propanoate are obtained in the form of an oil.

### 26.2: Methyl (S)-3-amino-2-[4-(propane-2-sulfonyl)piperazin-1-yl]propanoate dihydrochloride

In a manner analogous to Example 3.3, using 700 mg (1.8 mmol) of methyl (S)-3-tert-butoxycarbonylamino-2-[4-(propane-2-sulfonyl)piperazin-1-yl]propanoate, 620 mg (86%) of methyl (S)-3-amino-2-[4-(propane-2-sulfonyl)piperazin-1-yl]propanoate dihydrochloride are obtained in the form of an oil.

### 26.3: Methyl (S)-3-[4-(2-methylquinolin-4-ylmethoxy)benzenesulfonylamino]-2-[4-(propane-2-sulfonyl)piperazin-1-yl]propanoate

In a manner analogous to Example 17.6, using 620 mg (1.5 mmol) of methyl (S)-3-amino-2-[4-(propane-2-sulfonyl)piperazin-1-yl]propanoate dihydrochloride and 830 mg (2.1 mmol) of 4-(2-methylquinolin-4-ylmethoxy)benzenesulfonyl chloride hydrochloride (prepared as described in the example 17.2), 505 mg (54%) of methyl (S)-3-[4-(2-methylquinolin-4-ylmethoxy)benzenesulfonylamino]-2-[4-(propane-2-sulfonyl)piperazin-1-yl]propanoate are obtained in the form of a white solid.

### 26.4: (S)-3-[4-(2-methylquinolin-4-ylmethoxy)benzenesulfonylamino]-2-[4-(propane-2-sulfonyl)piperazin-1-yl]propanoic acid

In a manner analogous to Example 3.7, using 505 mg (0.8 mmol) of methyl (S)-3-[4-(2-methylquinolin-4-ylmethoxy)benzenesulfonylamino]-2-[4-(propane-2-sulfonyl)piperazin-1-yl]propanoate, 135 mg (27%) of (S)-3-[4-(2-methylquinolin-4-ylmethoxy)benzenesulfonylamino]-2-[4-(propane-2-sulfonyl)piperazin-1-yl]propanoic acid are obtained in the form of a white solid.

### 26.5: (S)—N-hydroxy-3-[4-(2-methylquinolin-4-ylmethoxy)benzenesulfonylamino]-2-[4-(propane-2-sulfonyl)piperazin-1-yl]propionamide

In a manner analogous to Example 3.8, using 135 mg (0.2 mmol) of (S)-3-[4-(2-methylquinolin-4-ylmethoxy)benzenesulfonylamino]-2-[4-(propane-2-sulfonyl)piperazin-1-yl]propanoic acid, 24 mg (17%) of (S)—N-hydroxy-3-[4-(2-methylquinolin-4-ylmethoxy)benzenesulfonylamino]-2-[4-(propane-2-sulfonyl)piperazin-1-yl]propionamide are obtained in the form of a white solid.

¹H NMR (δ, DMSO): 1.19 (d, J=6.8 Hz, 6H); 2.45 (m, 4H); 2.68 (s, 3H); 2.80-2.90 (m, 1H); 2.95-3.15 (m, 6H); 3.29 (m, 1H); 5.72 (s, 2H); 7.34 (d, J=8.9 Hz, 2H); 7.52 (m, 1H); 7.57 (m, 2H); 7.76-7.80 (m, 3H); 7.98 (d, J=8.2 Hz, 1H); 8.10 (d, J=8.1 Hz, 1H); 8.93 (s, 1H); 10.66 (s, 1H).

### Example 27

### (S)-2-(4-benzylpiperazin-1-yl)-N-hydroxy-3-[4-(2-trifluoromethylpyrazolo[1,5-a]pyridin-3-ylmethoxy)benzenesulfonylamino]propionamide

### 27.1: 4-Hydroxybenzenesulfonyl chloride

A solution of 7 g (30 mmol) of the sodium salt of 4-hydroxybenzenesulfonic acid dihydrate in 40 ml of dimethylformamide is added dropwise to a solution of 15.5 ml (181 mmol) of oxalyl chloride in 120 ml of dichloromethane cooled to −30° C. The reaction medium is slowly brought back to ambient temperature and then stirred at ambient temperature for 18 h. After the addition of 200 ml of ice, the reaction medium is extracted with ethyl acetate. The organic phase is washed with water and with a saturated aqueous solution of sodium chloride, dried over magnesium sulfate, filtered, and concentrated. 6.2 g (100%) of 4-hydroxybenzenesulfonyl chloride are obtained in the form of a colorless oil.

### 27.2: Methyl (S)-2-(4-benzylpiperazin-1-yl)-3-(4-hydroxybenzenesulfonylamino)propanoate

In a manner analogous to Example 3.6, using 5.8 g (30 mmol) of 4-hydroxybenzenesulfonyl chloride and 7.7 g (20 mmol) of methyl (S)-3-amino-2-(4-benzylpiperazin-1-yl)propanoate trihydrochloride (prepared as described in example 17.5), 2.25 g (27%) of methyl (S)-2-(4-benzylpiperazin-1-yl)-3-(4-hydroxybenzenesulfonylamino)propanoate are obtained in the form of a white solid.

### 27.3: Methyl (S)-2-(4-benzylpiperazin-1-yl)-3-[4-(2-trifluoromethylpyrazolo[1,5-a]pyridin-3-ylmethoxy)benzenesulfonylamino]propanoate

In a manner analogous to example 11.1, using 500 mg (1.1 mmol) of methyl (S)-2-(4-benzylpiperazin-1-yl)-3-(4-hydroxybenzenesulfonylamino)propanoate and 370 mg (1.7 mmol) of (2-trifluoromethylpyrazolo[1,5-a]pyridin-3-yl)methanol (prepared as described in example 25.2), 350 mg (50%) of methyl (S)-2-(4-benzylpiperazin-1-yl)-3-[4-(2-trifluoromethylpyrazolo[1,5-a]pyridin-3-ylmeth-oxy)benzenesulfonylamino]propanoate are obtained in the form of a colorless oil.

### 27.4: (S)-2-(4-benzylpiperazin-1-yl)-3-[4-(2-trifluoromethylpyrazolo[1,5-a]pyridin-3-ylmethoxy)benzenesulfonylamino]propanoic acid

In a manner analogous to Example 3.7, using 350 mg (0.5 mmol) of methyl (S)-2-(4-benzylpiperazin-1-yl)-3-[4-(2-trifluoromethylpyrazolo[1,5-a]pyridin-3-ylmethoxy)benzenesulfonylamino]propanoate, 165 mg (48%) of (S)-2-(4-benzylpiperazin-1-yl)-3-[4-(2-trifluoromethylpyrazolo[1,5-a]pyridin-3-ylmethoxy)benzenesulfonylamino]propanoic acid are obtained in the form of a white solid.

### 27.5: (S)-2-(4-benzylpiperazin-1-yl)-N-hydroxy-3-[4-(2-trifluoromethylpyrazolo[1,5-a]pyridin-3-ylmethoxy)benzenesulfonylamino]propionamide

In a manner analogous to Example 3.8, using 165 mg (0.3 mmol) of (S)-2-(4-benzylpiperazin-1-yl)-3-[4-(2-trifluoromethyl pyrazolo[1,5-a]pyridin-3-ylmethoxy)benzenesulfonylamino]propanoic acid, 50 mg (29%) of (S)-2-(4-benzylpiperazin-1-yl)-N-hydroxy-3-[4-(2-trifluoromethylpyrazolo[1,5-a]pyridin-3-ylmethoxy)benzenesulfonylamino]propionamide are obtained in the form of a white solid with a melting point of 138° C.

¹H NMR (δ, DMSO): 2.20 (m, 4H); 2.38 (m, 4H); 2.65-2.75 (m, 1H); 2.86-2.98 (m, 2H); 3.35 (m, 2H); 5.37 (s, 2H); 7.10-7.25 (m, 8H); 7.35-7.44 (m, 2H); 7.68 (d, J=8.9 Hz, 2H); 7.98 (d, J=9 Hz, 1H); 8.81 (m, 2H); 10.52 (s, 1H).

### Example 28

### Enzymatic Assay for TACE Inhibition

### Description of the Assay

The products are solubilized in DMSO at a concentration of 10 mM. A serial 3-fold dilution over 10 points is carried out so as to have a concentration range of from 10 μM to 0.5 nM final concentration.

The TACE enzyme is an internal production (carried out according to the publication “protein Eng Des Sel 2006, 19, 155-161”) and is added so as to have a signal equivalent to 6 times the background noise in 2 h at 37° C. The reaction is carried out in 50 mM Tris buffered medium containing 4% glycerol, pH 7.4. The fluorescent substrate is MCA-Pro-Leu-Ala-Val-(Dpa)-Arg-Ser-Ser-Arg-NH₂ (R&D systems, reference: ES003). The substrate is cleaved by the enzyme between the alanine and the valine, thus releasing a fluorescent peptide (excitation: 320 nm, emission: 420 nm). The substrate is used at 40 μM. The reaction is carried out in a final volume of 10 μl (4 μl inhibitor, 4 μl substrate, 2 μl enzyme) in a low volume 384-well plate (Corning reference: 3676). The plate is incubated at ambient temperature for 2 h, and then read by fluorescence on a Pherastar reader (BMG labtech). The IC₅₀ is determined using mathematical processing software (XLfit).

TABLE 1
Product assay

| Example No. | % TACE inhibition at 10 μM | IC50 - TACE (nM) |
|---|---|---|
| ex1 | 100 | 87 |
| ex2 | 100 | 32 |
| ex4 | 95 | 497 |
| ex5 | 99 | 21 |
| ex6 | 99 | 52 |
| ex8 | 100 | 127 |
| ex9 | 100 | 147 |
| ex10 | 93 | 47 |
| ex11 | 93 | 24 |
| ex13 | 96 | 108 |
| ex14 | 98 | 64 |
| ex16 | 96 | 168 |
| ex17 | 91 | 62 |
| ex18 | 90 | 67 |
| ex19 | 92 | 41 |
| ex21 | 97 | 63 |
| ex23 | 97 | 53 |
| ex24 | 98 | 86 |
| ex26 | 98 | 33 |

On the basis of the results obtained in the TACE enzymatic assay described above, the compounds provided herein are TNFα converting enzyme (TACE) inhibitors and consequently may be potential active ingredients for the treatment of pathological conditions for which reducing TNFα production would be of great interest.

### Example 29

### Selectivity Assay

Principle of the Assay:

The molecules are dose-response tested on the following enzymes: MMP-1, MMP-3, MMP-9, ADAM9 and ADAM10, according to the same protocol as that described for the TACE enzyme in Example 28, but with different substrates (MMP R&D systems, reference: P126-990, and ADAM R&D systems, reference: ES003).

The enzymes are purchased from Calbiochem.

TABLE 2
Product Assay

| | IC50 (nM) | | | | | |
|---|---|---|---|---|---|---|
| Example | MMP-1 | MMP-3 | MMP-9 | ADAM9 | ADAM10 | TACE |
| 5 | 5100 | 3200 | >10000 | >10000 | >10000 | 21 |
| 18 | 670 | 849 | >10000 | 9254 | >10000 | 67 |
| 19 | 2303 | 1770 | >10000 | 3054 | >10000 | 41 |
| 20 | 3935 | 4775 | >10000 | >10000 | >10000 | 140 |
| 21 | 1166 | 887 | >10000 | >10000 | >10000 | 63 |
| 23 | 2221 | 1065 | >10000 | >10000 | >10000 | 53 |
| 24 | 2059 | 1878 | >10000 | >10000 | >10000 | 86 |
| 26 | 969 | 574 | >10000 | >10000 | >10000 | 16 |
| Apratastat | 145 | 10 | 82 | 85 | 71 | 5 |

On the basis of the results obtained in the selectivity assay described above, these compounds are also very selective for TACE compared with the other ADAMs and MMPs, i.e. they have IC₅₀ values for other ADAMs or MMPs that are at least 10 times higher than that obtained for TACE, and more advantageously at least 100 times higher.

As it happens, insofar as it is known that the nonselective inhibition of these families of enzymes induces adverse side effects observed in vivo, the selective inhibition of TACE compared with these other enzymes should make it possible to reduce adverse side effects when these molecules are administered for the treatment of pathological conditions for which reducing TNFα production would be of great interest.

### Example 30

### Inhibition of MMPs

Several compounds were dose-response tested on TACE and MMP-1, MMP-3, and MMP-12 according to the same protocol as that described above. The structures of compounds A, B, C, and D are shown below. The results of the assay are summarized in the table below.

[chemical structure]

TABLE 3

| | Compound | | | |
|---|---|---|---|---|
| | A | B | C | D |
| TACE = ADAM 17 (IC₅₀ nM) | 25 | 51 | 62 | 33 |
| MMP-1 (IC₅₀ nM) | 3000 | 1983 | 216 | 969 |
| MMP-3 (IC₅₀ nM) | 4500 | 3034 | 317 | 574 |
| MMP-12 (IC₅₀ nM) | 977 | 226 | 22 | 29 |

As shown in Table 3, Compounds C and D were the most effective in inhibiting TACE, MMP-1, MMP-3, and MMP-12. Although Compounds A and B were very potent TACE inhibitors (IC₅₀ 25 nM and 51 nM), they showed limited effect on MMP inhibition with IC₅₀>1000 nM (with the exception of compound B on MMP12), and no effect on other MMPs (data not shown). Therefore, Compounds A & B were mainly selective inhibitors for TACE. In contrast, Compounds C and D, which were also potent TACE inhibitors (IC₅₀ of 62 nM and 33 nM), were efficient in inhibiting three particular metalloproteinases, MMP1, MMP3 and MP12 with IC₅₀<1000 nM. Moreover, Compounds C and D were as potent in inhibiting MMP12 as in inhibiting TACE. Therefore, these two compounds represent a particular class of molecules with a potent TACE inhibition and an intermediate selectivity to TACE. Particularly interesting is the fact that Compounds C and D inhibited only the 3 MMPs related to acne physiopathology: MMP1 MMP3 and MMP12. Therefore, they are attractive molecules with an expected higher clinical benefit and limited expected side effects.

### Example 31

### TACE Inhibitor for Reducing Inflammation Induced by P. acnes

P. acnes stimulates the production of inflammatory cytokines, such as TNFα and interleukins. In this Example, the mouse ear edema model was used to investigate inflammation induced by P. acnes. This is a chronic inflammation model involving intense innate and adaptive immunity. The thickness of the ear is measured each day to determine the amount of swelling caused by P. acnes. The amount of TNFα and IL-6 secreted is determined.

After one intradermal injection of live P. acnes on Day 0, each of the following preparations was topically applied once a day on the ear of a Balb/c mouse over the course of 7 days (from Day 1 to Day 7).

1) PBS and vehicle 173, once a day (no live P. acnes intradermal injection at Day 0)
2) Vehicle 173, once a day, after live P. acnes intradermal injection at Day 0
3) 0.01% CD0153F (001) once a day, after live P. acnes intradermal injection at Day 0
4) 0.1% Compound D (veh.173) once a day, after live P. acnes intradermal injection at Day 0
5) 0.3% Compound D (veh.173), once a day, after live P. acnes intradermal injection at Day 0
6) 0.6% Compound D (veh.173), once a day, after live P. acnes intradermal injection at Day 0
7) 0.6% Compound D (veh.173), twice a day, after live P. acnes intradermal injection at Day 0

Preparation 1 containing PBS and Vehicle 173 (acetone/citrate buffer (9/1) at pH 3.2) was used as a control. Preparation 2 containing CD0153F was used as a positive control. CD0153F is betamethasone valerate, a highly potent glucocorticoid steroid with anti-inflammatory properties, and 001 is the vehicle (acetone) used to dissolve CD0153F

Preparations 1-3 served as controls for comparison with preparations 4-7. Preparations 4-6 containing TACE antagonist Compound D in different amounts were administered once a day from day 1 to day 7, and preparation 7 containing TACE antagonist Compound D was administered twice a day from day 1 to day 7. The thickness of the ear was measured each day with a caliper from day 1 until day 8. The amount of TNFα and IL-6 secreted by the skin cells was determined using tissue biopsy samples from the mouse ear. TNF-α and Il-6 were measured using the mouse BD™ Cytometric Bead Array Flex (BD Bioscience, Dosage by FacsArray).

31.1: Cutaneous Inflammation Induced by P. acnes

The results of cutaneous inflammation in the ears of mice induced by P. acnes are shown in FIG. 1. Live P. acnes induced inflammation in the ears of mice within 24 hours (see preparation 2). CD0153F was used as a positive control to show inhibition of inflammation induced by P. acnes (see preparation 3). It appears that Compound D did not inhibit the edema induced by P. acnes, since the swelling was not reduced in the presence of TACE antagonist, Compound D, (preparations 4-7), and swelling was either at the same level or more than that for the control sample (preparations 2) which did not contain a TACE antagonist. Although the results indicated that only very strong pharmacological molecules targeting a large panel of inflammatory pathways such as the betamethasone valerate, were able to inhibit the clinical signs of the acute inflammation related to this model, it is likely that more selective pharmacological molecule will be able to reduce the level of inflammation at the clinical level in the acne physiopathology during which less pronounced inflammation does occur. Nevertheless, as shown below, Compound D was able to strongly inhibit the TNFα secreted during the acute inflammation occurring in this model, suggesting that the compounds provided herein will be even more potent in interfering with the TNFα pathway during acne physiopathology.

31.2: TNFα and IL-6 Secretion by Skin Cells

As shown in FIGS. 2A and 2B, P. acnes induced secretion of TNFα and IL-6 by ear skin cells of mice (see preparation 2). The data presented in FIGS. 2A and 2B confirmed that Compound D greatly reduced the secretion of TNFα and moderately reduced the secretion of IL-6 induced by P. acnes. The data demonstrate that Compound D used in an in vivo acute skin inflammation model, showed a very high efficiency in inhibiting TNFα secretion. As expected from this TNFα inhibition, a subsequent effect is the partial inhibition of IL6 secretion, IL6 secretion being in part regulated by the level of TNFα. Interestingly, Compound D is as potent as CD0153 in inhibiting TNFα secretion in this acute skin inflammation model.

Thus, although the inhibition of TNFα secretion by Compound D was not sufficient to inhibit edema induced by P. acnes by intradermal injection, Compound D significantly inhibited TNFα production induced by P. acnes in an in vivo model of acute and chronic skin inflammation. The results suggest that compound D will be even more efficient in blocking the TNFα pathway involved in the inflammation occurring during papule formation in acne lesions.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method of treating acne, the method comprising administering a pharmaceutical composition comprising an effective amount of a compound, a salt thereof, or an enantiomer thereof to a subject in need thereof, wherein the compound has a structure of formula (I) [chemical structure]
in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an alkyl radical, a substituted alkyl radical, an alkenyl radical, a substituted alkenyl radical, an alkynyl radical, a substituted alkynyl radical, an aryl radical, a substituted aryl radical, an aralkyl radical, a substituted aralkyl radical, a heterocyclic radical, a substituted heterocyclic radical, a cycloalkyl radical, a substituted cycloalkyl radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical; and
n can take the values of 0, 1, 2, or 3.

2. The method as claimed in claim 1, wherein the compound has the structure of formula (I), in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical; and
n can take the values of 0, 1 or 2.

3. The method as claimed in claim 1, wherein the compound has the structure of formula (I), in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a —C(O)—R₄ radical, or an —SO₂—R₄ radical, wherein R₄ is as defined below;
R₂ is a hydrogen atom or a lower alkyl radical;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical; and
n can take the values of 1 or 2.

4. The method as claimed in claim 1, wherein the compound has the structure of formula (I), in which:
R₁ is an alkyl radical, a substituted alkyl radical, an aralkyl radical, a substituted aralkyl radical, a —C(O)—R₄ radical, or an —SO₂—R₄ radical, wherein R₄ is as defined below;
R₂ is a hydrogen atom;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical; and
n takes the value of 1.

5. The method as claimed in claim 1, wherein the compound has the structure of formula (I), in which:
R₁ is an alkyl radical, a substituted alkyl radical, an aralkyl radical, a substituted aralkyl radical, a —C(O)—R₄ radical, or an —SO₂—R₄ radical, wherein R₄ is as defined below;
R₂ is a hydrogen atom;
R₃ is a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical; and
n takes the value of 1.

6. The method as claimed in claim 1, wherein the compound has the structure of formula (I), in which:
R₁ is an alkyl radical, a substituted alkyl radical, an aralkyl radical, a substituted aralkyl radical, a —C(O)—R₄ radical, or an —SO₂—R₄ radical, wherein R₄ is as defined below;
R₂ is a hydrogen atom;
R₃ is a heteroaryl radical or a substituted heteroaryl radical;
R₄ is an alkyl radical, a substituted alkyl radical, an aryl radical, a substituted aryl radical, an aralkyl radical, or a substituted aralkyl radical; and
n takes the value of 1.

7. The method as claimed in claim 1, wherein the compound is selected from the group consisting of:
1) 3-[(4-but-2-ynyloxybenzenesulfonyl)methylamino]-N-hydroxy-2-(4-methanesulfonylpiperazin-1-yl)propionamide;
2) (S)-3-(4-but-2-ynyloxybenzenesulfonylamino)-N-hydroxy-2-(4-methanesulfonylpiperazin-1-yl)propionamide;
3) (S)-3-(4-benzyloxybenzenesulfonylamino)-N-hydroxy-2-(4-methanesulfonylpiperazin-1-yl)propionamide;
4) (S)-3-[(4-benzyloxybenzenesulfonyl)methylamino]-N-hydroxy-2-(4-methanesulfonylpiperazin-1-yl)propionamide;
5) (S)—N-hydroxy-2-(4-methanesulfonylpiperazin-1-yl)-3-[4-(2-methylquinolin-4-ylmethoxy)benzenesulfonylamino]propionamide;
6) (S)—N-hydroxy-2-(4-methanesulfonylpiperazin-1-yl)-3-[4-(naphthalen-1-ylmethoxy)benzenesulfonylamino]propionamide;
7) (S)—N-hydroxy-2-(4-methanesulfonylpiperazin-1-yl)-3-(4-propoxybenzenesulfonylamino)propionamide;
8) (S)-3-[4-(3-cyanobenzyloxy)benzenesulfonylamino]-N-hydroxy-2-(4-methanesulfonylpiperazin-1-yl)propionamide;
9) (S)-3-[4-(4-cyanobenzyloxy)benzenesulfonylamino]-N-hydroxy-2-(4-methanesulfonylpiperazin-1-yl)propionamide;
10) benzyl 4-{(S)-1-hydroxycarbamoyl-2-[4-(2-methylquinolin-4-ylmethoxy)benzenesulfonylamino]ethyl}piperazine-1-carboxylate
11) (S)—N-hydroxy-2-(4-methanesulfonylpiperazin-1-yl)-3-[4-(2-phenylpyridin-4-ylmethoxy)benzenesulfonylamino]propionamide;
12) (R)—N-hydroxy-2-(4-methanesulfonylpiperazin-1-yl)-3-[4-(2-methylquinolin-4-ylmethoxy)benzenesulfonylamino]propionamide;
13) (S)—N-hydroxy-3-[4-(2-methylquinolin-4-ylmethoxy)benzenesulfonylamino]-2-piperazin-1-yl-propionamide;
14) (S)—N-hydroxy-2-(4-methanesulfonylpiperazin-1-yl)-3-[4-(2-methylquinolin-4-ylmethoxy)benzenesulfonylamino]propionamide hydrochloride;
15) tert-butyl 3-{4-[(S)-2-hydroxycarbamoyl-2-(4-methanesulfonylpiperazin-1-yl)ethylsulfamoyl]phenoxymethyl}-2-methylindole-1-carboxylate di(trifluoroacetate);
16) (S)—N-hydroxy-2-(4-methanesulfonylpiperazin-1-yl)-3-[4-(quinolin-4-ylmethoxy)benzenesulfonylamino]propionamide;
17) (S)-2-(4-benzylpiperazin-1-yl)-N-hydroxy-3-[4-(2-methylquinolin-4-ylmethoxy)benzenesulfonylamino]propionamide;
18) (S)-2-[4-(4-fluorobenzyl)piperazin-1-yl]-N-hydroxy-3-[4-(2-methylquinolin-4-ylmethoxy)benzenesulfonylamino]propionamide;
19) (S)-2-(4-ethylpiperazin-1-yl)-N-hydroxy-3-[4-(2-methylquinolin-4-ylmethoxy)benzenesulfonylamino]propionamide;
20) (S)—N-hydroxy-3-[4-(2-methylquinolin-4-ylmethoxy)benzenesulfonylamino]-2-[4-(4-trifluoromethyl-benzyl)piperazin-1-yl]propionamide;
21) (S)—N-hydroxy-2-[4-(4-methylbenzyl)piperazin-1-yl]-3-[4-(2-methylquinolin-4-ylmethoxy)benzenesulfonylamino]propionamide;
22) (S)-3-[4-(benzoisoxazol-3-ylmethoxy)benzenesulfonylamino]-N-hydroxy-2-(4-methanesulfonylpiperazin-1-yl)propionamide;
23) (S)—N-hydroxy-2-(4-isobutyrylpiperazin-1-yl)-3-[4-(2-methylquinolin-4-ylmethoxy)benzenesulfonylamino]propionamide;
24) (S)—N-hydroxy-2-[4-(2-methylpropane-1-sulfonyl)piperazin-1-l]-3-[4-(2-methylquinolin-4-ylmethoxy)benzenesulfonylamino]propionamide;
25) (S)—N-hydroxy-2-(4-methanesulfonylpiperazin-1-yl)-3-[4-(2-trifluoromethylpyrazolo[1,5-a]pyridin-3-ylmethoxy)benzenesulfonylamino]propionamide;
26) (S)—N-hydroxy-3-[4-(2-methylquinolin-4-ylmethoxy)benzenesulfonylamino]-2-[4-(propane-2-sulfonyl)piperazin-1-yl]propionamide;
27) (S)-2-(4-benzylpiperazin-1-yl)-N-hydroxy-3-[4-(2-trifluoromethylpyrazolo[1,5-a]pyridin-3-ylmethoxy)benzenesulfonylamino]propionamide;
28) (S)-2-(4-acetylpiperazin-1-yl)-N-hydroxy-3-[4-(2-methylquinolin-4-ylmethoxy)benzenesulfonylamino]propionamide;
29) (S)—N-hydroxy-2-(4-methanesulfonylpiperazin-1-yl)-3-{propyl-[4-(quinolin-4-ylmethoxy)benzenesulfonyl]amino}propionamide;
30) (S)-2-(4-benzenesulfonylpiperazin-1-yl)-N-hydroxy-3-[4-(pyrazolo[1,5-a]pyridin-3-ylmethoxy)benzenesulfonylamino]propionamide;
31) (S)-2-(4-benzylpiperazin-1-yl)-N-hydroxy-3-[4-(1-methylpiperidin-4-ylmethoxy)benzenesulfonylamino]propionamide;
32) (S)-2-[4-(4-fluorobenzoyl)piperazin-1-yl]-N-hydroxy-3-[4-(3-m-tolyl-propoxy)benzenesulfonylamino]propionamide;
33) (S)—N-hydroxy-3-[4-(2-methylnaphthalen-1-ylmethoxy)benzenesulfonylamino]-2-(4-propionylpiperazin-1-yl)propionamide;
34) (S)—N-hydroxy-3-[4-(4-methylpentyloxy)benzenesulfonylamino]-2-(4-phenylacetylpiperazin-1-yl)propionamide;
35) (S)—N-hydroxy-2-(4-methanesulfonylpiperazin-1-yl)-3-[4-(2-methylpyridin-4-ylmethoxy)benzenesulfonylamino]propionamide;
36) (S)-2-(3-acetylimidazolidin-1-yl)-N-hydroxy-3-[4-(2-methylquinolin-4-ylmethoxy)benzenesulfonylamino]propionamide;
37) (S)-3-[4-(3,5-dimethyl benzyloxy)benzenesulfonylamino]-N-hydroxy-2-imidazolidin-1-yl-propionamide;
38) (S)—N-hydroxy-2-(4-methanesulfonyl-[1,4]diazepan-1-yl)-3-[4-(2-methylquinolin-4-ylmethoxy)benzenesulfonylamino]propionamide;
39) (S)-2-(4-benzyl-[1,4]diazepan-1-yl)-N-hydroxy-3-[4-(2-methylquinolin-4-ylmethoxy)benzenesulfonylamino]propionamide;
40) (S)-2-[1,4]diazocan-1-yl-N-hydroxy-3-[4-(2-methylquinolin-4-ylmethoxy)benzenesulfonylamino]propionamide;
41) (S)—N-hydroxy-3-[4-(2-methylbenzofuran-3-ylmethoxy)benzenesulfonylamino]-2-[4-(propane-2-sulfonyl)piperazin-1-yl]propionamide; and
42) (S)-2-(4-benzylpiperazin-1-yl)-N-hydroxy-3-[4-(2-isopropyl-1H-indol-3-ylmethoxy)benzenesulfonylamino]propionamide.

8. The method of claim 1, wherein the compound is (S)—N-hydroxy-3-[4-(2-methylquinolin-4-ylmethoxy)benzenesulfonylamino]-2-[4-(propane-2-sulfonyl)piperazin-1-yl]propionamide.

9. A method of inhibiting inflammation induced by P. acnes, the method comprising administering a pharmaceutical composition comprising an effective amount of a compound, a salt thereof, or an enantiomer thereof to a subject infected with P. acnes, wherein the compound has a structure of formula (I) [chemical structure]
in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an alkyl radical, a substituted alkyl radical, an alkenyl radical, a substituted alkenyl radical, an alkynyl radical, a substituted alkynyl radical, an aryl radical, a substituted aryl radical, an aralkyl radical, a substituted aralkyl radical, a heterocyclic radical, a substituted heterocyclic radical, a cycloalkyl radical, a substituted cycloalkyl radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical; and
n can take the values of 0, 1, 2, or 3.

10. The method as claimed in claim 9, wherein the compound has the structure of formula (I), in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical; and
n can take the values of 0, 1 or 2.

11. The method as claimed in claim 9, wherein the compound has the structure of formula (I), in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a —C(O)—R₄ radical, or an —SO₂—R₄ radical, wherein R₄ is as defined below;
R₂ is a hydrogen atom or a lower alkyl radical;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical; and
n can take the values of 1 or 2.

12. The method as claimed in claim 9, wherein the compound has the structure of formula (I), in which:
R₁ is an alkyl radical, a substituted alkyl radical, an aralkyl radical, a substituted aralkyl radical, a —C(O)—R₄ radical, or an —SO₂—R₄ radical, wherein R₄ is as defined below;
R₂ is a hydrogen atom;
R₃ is an aryl radical, a substituted aryl radical, an aralkyl radical, a substituted aralkyl radical, a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical; and
n takes the value of 1.

13. The method as claimed in claim 9, wherein the compound has the structure of formula (I), in which:
R₁ is an alkyl radical, a substituted alkyl radical, an aralkyl radical, a substituted aralkyl radical, a —C(O)—R₄ radical, or an —SO₂—R₄ radical, wherein R₄ is as defined below;
R₂ is a hydrogen atom;
R₃ is a heterocyclic radical, a substituted heterocyclic radical, a heteroaryl radical, a substituted heteroaryl radical, a heteroaralkyl radical, or a substituted heteroaralkyl radical;
R₄ is an alkyl radical, a substituted alkyl radical, an aryl radical, a substituted aryl radical, an aralkyl radical, or a substituted aralkyl radical; and
n takes the value of 1.

14. The method as claimed in claim 9, wherein the compound has the structure of formula (I), in which:
R₁ is an alkyl radical, a substituted alkyl radical, an aralkyl radical, a substituted aralkyl radical, a —C(O)—R₄ radical, or an —SO₂—R₄ radical, wherein R₄ is as defined below;
R₂ is a hydrogen atom;
R₃ is a heteroaryl radical or a substituted heteroaryl radical;
R₄ is an alkyl radical, a substituted alkyl radical, an aryl radical, a substituted aryl radical, an aralkyl radical, or a substituted aralkyl radical; and
n takes the value of 1.

15. The method as claimed in claim 9, wherein the compound is selected from the group consisting of:
1) 3-[(4-but-2-ynyloxybenzenesulfonyl)methylamino]-N-hydroxy-2-(4-methanesulfonylpiperazin-1-yl)propionamide;
2) (S)-3-(4-but-2-ynyloxybenzenesulfonylamino)-N-hydroxy-2-(4-methanesulfonylpiperazin-1-yl)propionamide;
3) (S)-3-(4-benzyloxybenzenesulfonylamino)-N-hydroxy-2-(4-methanesulfonylpiperazin-1-yl)propionamide;
4) (S)-3-[(4-benzyloxybenzenesulfonyl)methylamino]-N-hydroxy-2-(4-methanesulfonylpiperazin-1-yl)propionamide;
5) (S)—N-hydroxy-2-(4-methanesulfonylpiperazin-1-yl)-3-[4-(2-methylquinolin-4-ylmethoxy)benzenesulfonylamino]propionamide;
6) (S)—N-hydroxy-2-(4-methanesulfonylpiperazin-1-yl)-3-[4-(naphthalen-1-ylmethoxy)benzenesulfonylamino]propionamide;
7) (S)—N-hydroxy-2-(4-methanesulfonylpiperazin-1-yl)-3-(4-propoxybenzenesulfonylamino)propionamide;
8) (S)-3-[4-(3-cyanobenzyloxy)benzenesulfonylamino]-N-hydroxy-2-(4-methanesulfonylpiperazin-1-yl)propionamide;
9) (S)-3-[4-(4-cyanobenzyloxy)benzenesulfonylamino]-N-hydroxy-2-(4-methanesulfonylpiperazin-1-yl)propionamide;
10) benzyl 4-{(S)-1-hydroxycarbamoyl-2-[4-(2-methylquinolin-4-ylmethoxy)benzenesulfonylamino]ethyl}piperazine-1-carboxylate 11) (S)—N-hydroxy-2-(4-methanesulfonylpiperazin-1-yl)-3-[4-(2-phenylpyridin-4-ylmethoxy)benzenesulfonylamino]propionamide;
12) (R)—N-hydroxy-2-(4-methanesulfonylpiperazin-1-yl)-3-[4-(2-methylquinolin-4-ylmethoxy)benzenesulfonylamino]propionamide;
13) (S)—N-hydroxy-3-[4-(2-methylquinolin-4-ylmethoxy)benzenesulfonylamino]-2-piperazin-1-yl-propionamide;
14) (S)—N-hydroxy-2-(4-methanesulfonylpiperazin-1-yl)-3-[4-(2-methylquinolin-4-ylmethoxy)benzenesulfonylamino]propionamide hydrochloride;
15) tert-butyl 3-{4-[(S)-2-hydroxycarbamoyl-2-(4-methanesulfonylpiperazin-1-yl)ethylsulfamoyl]phenoxymethyl}-2-methylindole-1-carboxylate di(trifluoroacetate);
16) (S)—N-hydroxy-2-(4-methanesulfonylpiperazin-1-yl)-3-[4-(quinolin-4-ylmethoxy)benzenesulfonylamino]propionamide;
17) (S)-2-(4-benzylpiperazin-1-yl)-N-hydroxy-3-[4-(2-methylquinolin-4-ylmethoxy)benzenesulfonylamino]propionamide;
18) (S)-2-[4-(4-fluorobenzyl)piperazin-1-yl]-N-hydroxy-3-[4-(2-methylquinolin-4-ylmethoxy)benzenesulfonylamino]propionamide;
19) (S)-2-(4-ethylpiperazin-1-yl)-N-hydroxy-3-[4-(2-methylquinolin-4-ylmethoxy)benzenesulfonylamino]propionamide;
20) (S)—N-hydroxy-3-[4-(2-methylquinolin-4-ylmethoxy)benzenesulfonylamino]-2-[4-(4-trifluoromethyl-benzyl)piperazin-1-yl]propionamide;
21) (S)—N-hydroxy-2-[4-(4-methylbenzyl)piperazin-1-yl]-3-[4-(2-methylquinolin-4-ylmethoxy)benzenesulfonylamino]propionamide;
22) (S)-3-[4-(benzoisoxazol-3-ylmethoxy)benzenesulfonylamino]-N-hydroxy-2-(4-methanesulfonylpiperazin-1-yl)propionamide;
23) (S)—N-hydroxy-2-(4-isobutyrylpiperazin-1-yl)-3-[4-(2-methylquinolin-4-ylmethoxy)benzenesulfonylamino]propionamide;
24) (S)—N-hydroxy-2-[4-(2-methylpropane-1-sulfonyl)piperazin-1-l]-3-[4-(2-methylquinolin-4-ylmethoxy)benzenesulfonylamino]propionamide;
25) (S)—N-hydroxy-2-(4-methanesulfonylpiperazin-1-yl)-3-[4-(2-trifluoromethylpyrazolo[1,5-a]pyridin-3-ylmethoxy)benzenesulfonylamino]propionamide;
26) (S)—N-hydroxy-3-[4-(2-methylquinolin-4-ylmethoxy)benzenesulfonylamino]-2-[4-(propane-2-sulfonyl)piperazin-1-yl]propionamide;
27) (S)-2-(4-benzylpiperazin-1-yl)-N-hydroxy-3-[4-(2-trifluoromethylpyrazolo[1,5-a]pyridin-3-ylmethoxy)benzenesulfonylamino]propionamide;
28) (S)-2-(4-acetylpiperazin-1-yl)-N-hydroxy-3-[4-(2-methylquinolin-4-ylmethoxy)benzenesulfonylamino]propionamide;
29) (S)—N-hydroxy-2-(4-methanesulfonylpiperazin-1-yl)-3-{propyl-[4-(quinolin-4-ylmethoxy)benzenesulfonyl]amino}propionamide;
30) (S)-2-(4-benzenesulfonylpiperazin-1-yl)-N-hydroxy-3-[4-(pyrazolo[1,5-a]pyridin-3-ylmethoxy)benzenesulfonylamino]propionamide;
31) (S)-2-(4-benzylpiperazin-1-yl)-N-hydroxy-3-[4-(1-methylpiperidin-4-ylmethoxy)benzenesulfonylamino]propionamide;
32) (S)-2-[4-(4-fluorobenzoyl)piperazin-1-yl]-N-hydroxy-3-[4-(3-m-tolyl-propoxy)benzenesulfonylamino]propionamide;
33) (S)—N-hydroxy-3-[4-(2-methylnaphthalen-1-ylmethoxy)benzenesulfonylamino]-2-(4-propionylpiperazin-1-yl)propionamide;
34) (S)—N-hydroxy-3-[4-(4-methylpentyloxy)benzenesulfonylamino]-2-(4-phenylacetylpiperazin-1-yl)propionamide;
35) (S)—N-hydroxy-2-(4-methanesulfonylpiperazin-1-yl)-3-[4-(2-methylpyridin-4-ylmethoxy)benzenesulfonylamino]propionamide;
36) (S)-2-(3-acetylimidazolidin-1-yl)-N-hydroxy-3-[4-(2-methylquinolin-4-ylmethoxy)benzenesulfonylamino]propionamide;
37) (S)-3-[4-(3,5-dimethylbenzyloxy)benzenesulfonylamino]-N-hydroxy-2-imidazolidin-1-yl-propionamide;
38) (S)—N-hydroxy-2-(4-methanesulfonyl-[1,4]diazepan-1-yl)-3-[4-(2-methylquinolin-4-ylmethoxy)benzenesulfonylamino]propionamide;
39) (S)-2-(4-benzyl-[1,4]diazepan-1-yl)-N-hydroxy-3-[4-(2-methylquinolin-4-ylmethoxy)benzenesulfonylamino]propionamide;
40) (S)-2-[1,4]diazocan-1-yl-N-hydroxy-3-[4-(2-methylquinolin-4-ylmethoxy)benzenesulfonylamino]propionamide;
41) (S)—N-hydroxy-3-[4-(2-methylbenzofuran-3-ylmethoxy)benzenesulfonylamino]-2-[4-(propane-2-sulfonyl)piperazin-1-yl]propionamide; and
42) (S)-2-(4-benzylpiperazin-1-yl)-N-hydroxy-3-[4-(2-isopropyl-1H-indol-3-ylmethoxy)benzenesulfonylamino]propionamide.

16. The method of claim 9, wherein the compound is (S)—N-hydroxy-3-[4-(2-methylquinolin-4-ylmethoxy)benzenesulfonylamino]-2-[4-(propane-2-sulfonyl)piperazin-1-yl]propionamide.

17. A method of treating acne, the method comprising administering a pharmaceutical composition comprising an effective amount of a compound, a salt thereof, or an enantiomer thereof to a subject in need thereof, wherein the compound is (S)—N-hydroxy-3-[4-(2-methylquinolin-4-ylmethoxy)benzenesulfonylamino]-2-[4-(propane-2-sulfonyl)piperazin-1-yl]propionamide.

18. A method of inhibiting inflammation induced by P. acnes, the method comprising administering a pharmaceutical composition comprising an effective amount of a compound, a salt thereof, or an enantiomer thereof to a subject infected with P. acnes, wherein the compound is (S)—N-hydroxy-3-[4-(2-methylquinolin-4-ylmethoxy)benzenesulfonylamino]-2-[4-(propane-2-sulfonyl)piperazin-1-yl]propionamide.

19. A method of inhibiting activation of a transcription factor in an acne lesion comprising applying a pharmaceutical composition comprising an effective amount of a compound, a salt thereof, or an enantiomer thereof, to a skin having acne lesions wherein the compound has a structure of formula (I), [chemical structure]
in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an alkyl radical, a substituted alkyl radical, an alkenyl radical, a substituted alkenyl radical, an alkynyl radical, a substituted alkynyl radical, an aryl radical, a substituted aryl radical, an aralkyl radical, a substituted aralkyl radical, a heterocyclic radical, a substituted heterocyclic radical, a cycloalkyl radical, a substituted cycloalkyl radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical; and
n can take the values of 0, 1, 2, or 3.

20. The method of claim 19, wherein the transcription factor is nuclear factor-KB or activator protein-1 (AP-1).

21. The method of claim 19, wherein the skin is a skin sample from a human patient.

22. The method of claim 19, wherein the skin is on a mammalian body.

23. The method of claim 20, wherein the method further comprises inhibiting production of one or more cytokines selected from the group consisting of TNFα and IL-6.

24. The method of claim 20, wherein the method further comprises inhibiting production of one or more MMPs selected from the group consisting of MMP-1 and MMP-3.

25. A method of inhibiting production of MMP-12, the method comprising applying a pharmaceutical composition comprising an effective amount of a compound, a salt thereof, or an enantiomer thereof, to a subject in need thereof, wherein the compound has a structure of formula (I), [chemical structure]
in which:
R₁ is a hydrogen, an alkyl radical, a substituted alkyl radical, an alkenyl radical, a substituted alkenyl radical, an alkynyl radical, a substituted alkynyl radical, an aralkyl radical, a substituted aralkyl radical, a heteroaralkyl radical, a substituted heteroaralkyl radical, a —C(O)—R₄ radical, a —SO₂—R₄ radical, or a —C(O)OR₄ radical, wherein R₄ is as defined below;
R₂ is a hydrogen atom or a lower alkyl radical;
R₃ is an alkyl radical, a substituted alkyl radical, an alkenyl radical, a substituted alkenyl radical, an alkynyl radical, a substituted alkynyl radical, an aryl radical, a substituted aryl radical, an aralkyl radical, a substituted aralkyl radical, a heterocyclic radical, a substituted heterocyclic radical, a cycloalkyl radical, a substituted cycloalkyl radical, a heteroaryl radical, a substituted heteroaryl radical, a heteroaralkyl radical, or a substituted heteroaralkyl radical;
R₄ is an alkyl radical, a substituted alkyl radical, an alkenyl radical, a substituted alkenyl radical, an alkynyl radical, a substituted alkynyl radical, an aryl radical, a substituted aryl radical, an aralkyl radical, or a substituted aralkyl radical; and
n can take the values of 0, 1, 2, or 3.
